SEQUENCE LISTING (23 sequences)

SEQ ID NO: 1 - RNA, 21 nt, synthetic construct
  source 1..21: mol_type = other RNA; note = Sense Strand
cctggacattcagaacaagaa

SEQ ID NO: 2 - RNA, 23 nt, synthetic construct
  source 1..23: mol_type = other RNA; note = Antisense Strand
ttcttgttctgaatgtccagggt

SEQ ID NO: 3 - RNA, 23 nt, synthetic construct
  source 1..23: mol_type = other RNA; note = Antisense Strand
ttcttgttctgaatgtccagggt

SEQ ID NO: 4 - RNA, 23 nt, synthetic construct
  source 1..23: mol_type = other RNA; note = Antisense Strand
ttcttgttctgaatgtccagggt

SEQ ID NO: 5 - RNA, 23 nt, synthetic construct
  source 1..23: mol_type = other RNA; note = Antisense Strand
ttcttgttctgaatgtccagggt

SEQ ID NO: 6 - RNA, 23 nt, synthetic construct
  source 1..23: mol_type = other RNA; note = Antisense Strand
ttcttgttctgaatgtccagggt

SEQ ID NO: 7 - RNA, 21 nt, synthetic construct
  source 1..21: mol_type = other RNA; note = Sense Strand
cacagaaactcaataaagtga

SEQ ID NO: 8 - RNA, 23 nt, synthetic construct
  source 1..23: mol_type = other RNA; note = Antisense Strand
tcactttattgagtttctgtgcc

SEQ ID NO: 9 - RNA, 23 nt, synthetic construct
  source 1..23: mol_type = other RNA; note = Antisense Strand
tcactttattgagtttctgtgcc

SEQ ID NO: 10 - RNA, 23 nt, synthetic construct
  source 1..23: mol_type = other RNA; note = Antisense Strand
tcactttattgagtttctgtgcc

SEQ ID NO: 11 - RNA, 23 nt, synthetic construct
  source 1..23: mol_type = other RNA; note = Antisense Strand
tcactttattgagtttctgtgcc

SEQ ID NO: 12 - RNA, 23 nt, synthetic construct
  source 1..23: mol_type = other RNA; note = Antisense Strand
tcactttattgagtttctgtgcc

SEQ ID NO: 13 - RNA, 23 nt, synthetic construct
  source 1..23: mol_type = other RNA; note = Antisense Strand
ttcttgttctgaatgtccagggt

SEQ ID NO: 14 - RNA, 23 nt, synthetic construct
  source 1..23: mol_type = other RNA; note = Antisense Strand
tcactttattgagtttctgtgcc

SEQ ID NO: 15 - DNA, 20 bp, synthetic construct
  source 1..20: mol_type = other DNA; note = primer
gaggcttgtcacccttcttt

SEQ ID NO: 16 - DNA, 21 bp, synthetic construct
  source 1..21: mol_type = other DNA; note = primer
gccttgtgagcaccagtatta

SEQ ID NO: 17 - DNA, 26 bp, synthetic construct
  source 1..26: mol_type = other DNA; note = primer
atccctcacttcccagggtgtagaat

SEQ ID NO: 18 - DNA, 23 bp, synthetic construct
  source 1..23: mol_type = other DNA; note = primer
aactacctggcttggatccagaa

SEQ ID NO: 19 - DNA, 19 bp, synthetic construct
  source 1..19: mol_type = other DNA; note = primer
cccaaggagcacacaagga

SEQ ID NO: 20 - DNA, 20 bp, synthetic construct
  source 1..20: mol_type = other DNA; note = primer
catattgcttcataactaac

SEQ ID NO: 21 - DNA, 20 bp, synthetic construct
  source 1..20: mol_type = other DNA; note = primer
cggcaaattcaacggcacag

SEQ ID NO: 22 - DNA, 20 bp, synthetic construct
  source 1..20: mol_type = other DNA; note = primer
ccacgacatactcagcaccg

SEQ ID NO: 23 - DNA, 27 bp, synthetic construct
  source 1..27: mol_type = other DNA; note = primer
accatcttccaggagcgagaccccact

(19) United States
(12) Patent Application Publication
(10) Pub. No.: US 2026/0176624 A1
(43) Pub. Date: Jun. 25, 2026

(54) PRODRUG FOR DELIVERING SIRNA INTO CELL

(71) Applicant: Rona Bioscience, Limited, Hong Kong (CN)

(72) Inventors: Jinyu Huang, Shanghai (CN); Hao Zou, Shanghai (CN); Junkai Liu, Shanghai (CN); Yang Ke, Shanghai (CN)

(73) Assignee: Rona Bioscience, Limited, Hong Kong (CN)

(21) Appl. No.: 19/125,450

(22) PCT Filed: Oct. 30, 2023
(86) PCT No.: PCT/CN2023/127941

§ 371 Date: Apr. 29, 2025

(30) Foreign Application Priority Data
Oct. 31, 2022 (CN) 202211374509.3
Feb. 9, 2023 (CN) 202310101722.5
May 16, 2023 (CN) 202310552663.3

(51) Int. Cl. C12N 15/113; A61K 31/713; C07B 59/00; C07H 21/02

(52) U.S. Cl. CPC C12N 15/113; A61K 31/713; C07B 59/005; C07H 21/02; C07B 2200/05; C12N 2310/14; C12N 2310/531

(57) ABSTRACT

The present invention provides an oligonucleotide, containing a compound of formula (Ia), or a pharmaceutically acceptable salt, tautomer, or stereoisomer thereof. The compound of formula (I) is contained inside the oligonucleotide or at the 5′ terminal and/or the 3′ terminal, and is used for improving the ability of double-stranded RNA to pass through a cell membrane. The present invention also relates to a double-stranded RNA, a cell, a pharmaceutical composition and a kit containing the oligonucleotide.

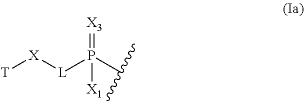

### CROSS REFERENCE TO RELATED APPLICATIONS

The present invention is a National Stage filing under 35 U.S.C. 371 of International PCT Application No. PCT/CN2023/127941, filed on Oct. 30, 2023, which claims the priority to Chinese Patent Application No. CN202211374509.3 filed on Oct. 31, 2022, Chinese Patent Application No. CN202310101722.5 filed on Feb. 9, 2023, and Chinese Patent Application No. CN202310552663.3 filed on May 16, 2023, which are incorporated herein by reference in their entirety as part of the disclosure of the present invention.

### REFERENCE TO SEQUENCE LISTING

The present application is being filed along with a Sequence Listing in electronic format. The Sequence Listing is provided as a file entitled 145954.05701—Sequence Listing.xml, created on Apr. 29, 2025, which is 25,096 bytes in size. The information in the electronic format of the sequence listing is incorporated herein by reference in its entirety.

### FIELD OF THE INVENTION

The present invention relates to the technical field of medical and pharmaceutical science, especially masking groups enhancing the cell membrane penetrating ability of dsRNA molecules, for example the T-X-L- group in the structure of formula (I), as well as a compound of formula (I) linking said masking groups to nucleotides, or a pharmaceutically acceptable salt, tautomer or stereoisomer thereof.

### BACKGROUND OF THE INVENTION

RNA interference is a phenomenon of specific and highly efficient degradation of the target mRNA induced by a double-stranded RNA (dsRNA).

It was found that, through preparing small interference RNA (siRNA) into a prodrug form, the intracellular cleavable masking groups can mask the negative charge of the phosphate groups of the oligonucleotide, thereby enhancing cell penetration. For example, WO2022147214A2 discloses a prodrug based on a disulfide-bond-containing ring.

However, the method of using prodrugs is still challenging, partially due to the difficulty of selecting the best masking group. For instance, the intracellular cleavage of masking groups will often lead to disadvantageous or even toxic products. Moreover, the masking group need to balance the absorption in the intestine and the cleavage in blood or target cells.

Therefore, more prodrugs are needed in the art for effectively delivering siRNAs in vivo.

### SUMMARY OF THE INVENTION

In one aspect, the present invention provides an oligonucleotide, comprising a compound of formula (II), or a pharmaceutically acceptable salt, tautomer or stereoisomer thereof:

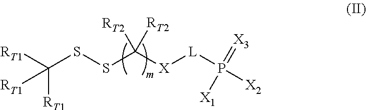

wherein each variable is defined as follows.

In another aspect, the present invention provides an oligonucleotide, comprising one, two or more compounds of formula (Ia), or a pharmaceutically acceptable salt, tautomer or stereoisomer thereof:

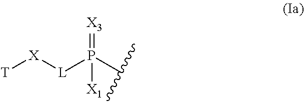

wherein each variable is defined as follows.

In another aspect, the present invention provides a dsRNA having a sense strand and an antisense strand, with each strand having 14-30 nucleotides; wherein the antisense strand comprise a sequence sufficiently complementary to the sense strand and target mRNA; wherein the sense strand and/or the antisense strand comprise one or more aforementioned compounds of formula (II) or aforementioned compounds of formula (Ia), or a pharmaceutically acceptable salt, tautomer or stereoisomer thereof.

In another aspect, the present invention provides a cell comprising the aforementioned dsRNA.

In another aspect, the present invention provides a pharmaceutical composition comprising the aforementioned dsRNA or the aforementioned cell and optionally a pharmaceutically acceptable carrier or excipient.

In another aspect, the present invention provides kit comprising the aforementioned dsRNA, the aforementioned cell or the aforementioned pharmaceutical composition.

In another aspect, the present invention provides a compound of formula (IIb), or a pharmaceutically acceptable salt, tautomer or stereoisomer thereof:

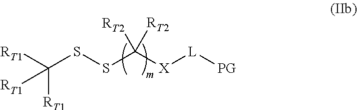

wherein each variable is defined as follows.

### DETAILED DESCRIPTION OF THE INVENTION

### Definitions

### Chemical Definition

Definitions of specific functional groups and chemical terms are described in more detail as follows.

When a numerical range is provided, it is intended that a particular numerical point and sub-range within said range be included. For example, “C₁₋₆ alkyl” includes alkyls C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

“C₁₋₆ alkyl” refers to any straight-chain or branched saturated hydrocarbon group with 1 to 6 carbon atoms. In some embodiments, C₁₋₄ alkyl and C₁₋₂ alkyl are preferred. Examples of C₁₋₆ alkyl described herein include, but are not limited to: methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentyl (C₅), pentyl (C₅), neopentyl (C₅), 3-methyl-2-butyl (C₅), tert-pentyl (C₅) and n-hexyl (C₆). The term “C₁₋₆ alkyl” also includes any heteroalkyl in which one or more (e.g., 1, 2, 3, or 4) carbon atoms are replaced with heteroatoms (e.g., oxygen, sulfur, nitrogen, boron, silicon, phosphorus). The alkyls may be optionally substituted by one or more substituents, for example, 1 to 5 substituents, 1 to 3 substituents, or 1 substituent. The conventional abbreviations for alkyl include: Me(—CH₃), Et(—CH₂CH₃), iPr(—CH(CH₃)₂), nPr(—CH₂CH₂CH₃), n-Bu(—CH₂CH₂CH₂CH₃) or i-Bu(—CH₂CH(CH₃)₂).

“C₂₋₆ alkenyl” refers to a straight-chain or branched hydrocarbon group with 2 to 6 carbon atoms and at least one carbon-carbon double bond. In some embodiments, C₂₋₄ alkenyl is preferred. Examples of C₂₋₆ alkenyl include, but are not limited to: vinyl (C₂), 1-propenyl (C₃), 2-propenyl (C₃), 1-butenyl (C₄), 2-butenyl (C₄), butadienyl (C₄), pentenyl (C₅), pentadienyl (C₅), and hexenyl (C₆). The term “C₂₋₆ alkenyl” also includes any heteroalkenyl in which one or more (e.g., 1, 2, 3, or 4) carbon atoms are replaced with heteroatoms (e.g., oxygen, sulfur, nitrogen, boron, silicon, phosphorus). The alkenyls may be optionally substituted by one or more substituents, for example, 1 to 5 substituents, 1 to 3 substituents, or 1 substituent.

“C₂₋₆ alkynyl” refers to a straight-chain or branched hydrocarbon group with 2 to 6 carbon atoms and at least one carbon-carbon triple bond and optionally one or more carbon-carbon double bonds. In some embodiments, C₂₋₄ alkynyl is preferred. Examples of C₂₋₆ alkynyl include, but are not limited to: ethynyl (C₂), 1-propynyl (C₃), 2-propynyl (C₃), 1-butynyl (C₄), 2-butynyl (C₄), pentynyl (C₅), and hexynyl (C₆). The term “C₂₋₆ alkynyl” also includes any heteroalkynyl in which one or more (e.g., 1, 2, 3, or 4) carbon atoms are replaced with heteroatoms (e.g., oxygen, sulfur, nitrogen, boron, silicon, phosphorus). The alkynyls may be optionally substituted by one or more substituents, for example, 1 to 5 substituents, 1 to 3 substituents, or 1 substituent.

“Halo-” or “halogen” refers to (substitution by) fluorine (F), chlorine (Cl), bromine (Br) and iodine (I).

Accordingly, “C₁₋₆ haloalkyl” refers to the aforementioned “C₁₋₆ alkyl”, with one or more halogen groups. In some embodiments, a C₁₋₄ haloalkyl is particularly preferred, and a C₁₋₂ haloalkyl is even more preferred. Exemplary haloalkyls include, but are not limited to: —CF₃, —CH₂F, —CHF₂, —CHFCH₂F, —CH₂CHF₂, —CF₂CF₃, —CCl₃, —CH₂Cl, —CHCl₂, and 2,2,2-trifluoro-1,1-dimethyl-ethyl. The haloalkyls may be substituted at any substitutable connection site, for example 1 to 5 substituents, 1 to 3 substituents or 1 substituent.

“C₃₋₁₀ cycloalkyl” refers to a non-aromatic cyclic hydrocarbon group with 3 to 10 ring carbon atoms and no heteroatoms. In some embodiments, C₄₋₇ cycloalkyl and C₃₋₆ cycloalkyl are preferred, more preferably C₅₋₆cycloalkyl. A cycloalkyl herein also includes a ring system in which an aforementioned cycloalkyl ring is fused with one or more aryls or heteroaryls through any connection site(s) on the cycloalkyl ring. In this context, the number of carbons still represents the number of carbons in the cycloalkyl system. Exemplary cycloalkyls include, but are not limited to: cyclopropyl (C₃), cyclopropanyl (C₃), Cyclobutyl (C₄), Cyclobutenyl (C₄), Cyclopentyl (C₅), Cyclopentenyl (C₅), Cyclohexyl (C₆), Cyclohexenyl (C₆), Cyclohexadienyl (C₆), Cycloheptyl (C₇), Cycloheptenyl (C₇), Cycloheptadienyl (C₇), Cycloheptatrienyl (C₇). The cycloalkyls may be optionally substituted by one or more substituents, for example, 1 to 5 substituents, 1 to 3 substituents, or 1 substituent.

“3-10-membered heterocyclyl” refers to a group of a 3-membered to 10-membered non-aromatic ring system with ring carbon atom(s) and 1 to 5 ring heteroatoms, wherein each heteroatom is independently selected from nitrogen, oxygen, sulfur, boron, phosphorus, and silicon. In said heterocyclyl containing one or more nitrogen atoms, the connection site may be a carbon or nitrogen atom as long as the valence permits. In some embodiments, 4-10-membered heterocyclyl is preferred, which is a 4-membered to 10-membered non-aromatic ring system with ring carbon atom(s) and 1 to 5 ring heteroatoms. In some embodiments, 3-8-membered heterocyclyl is preferred, which is a 3-membered to 8-membered non-aromatic ring system with ring carbon atom(s) and 1 to 4 ring heteroatoms; 4-7-membered heterocyclyl is preferred, which is a 4-membered to 7-membered non-aromatic ring system with ring carbon atom(s) and 1 to 3 ring heteroatoms; 5-6-membered heterocyclyl is more preferred, which is a 5-membered to 6-membered non-aromatic ring system with ring carbon atom(s) and 1 to 3 ring heteroatoms. A heterocyclyl herein also includes a ring system in which an aforementioned heterocyclyl ring is fused to one or more cycloalkyls through any connection site(s) on the cycloalkyl ring, or said heterocyclyl includes a ring system in which an aforementioned heterocyclyl ring is fused to one or more aryls or heteroaryls through any connection site(s) on the heterocyclyl ring. Under such cases, these contexts, the number of ring members still represents the number of ring members in the heterocyclyl ring system. Exemplary 3-membered heterocyclyls containing one heteroatom include, but are not limited to: aziridinyl, oxiranyl, and thiorenyl. Exemplary 4-membered heterocyclyls containing one heteroatom include, but are not limited to: azetidinyl, oxetidinyl, and thietanyl. Exemplary 5-membered heterocyclyls containing one heteroatom include, but are not limited to: tetrahydrofuryl, dihydrofuryl, tetrahydrothienyl, dihydrothienyl, pyrrolidinyl, dihydropyrrolyl, and pyrroli-2,5-dione. Exemplary 5-membered heterocyclyls containing two heteroatoms include, but are not limited to: dioxolanyl, oxasulfuranyl, disulfuranyl, and oxazolidin-2-one. Exemplary 5-membered heterocyclyls containing three heteroatoms include, but are not limited to: triazolinyl, oxadiazolinyl and thiadiazolinyl. Exemplary 6-membered heterocyclyls containing one heteroatom include, but are not limited to: piperidinyl, tetrahydropyranyl, dihydropyridyl and thianyl. Exemplary 6-membered heterocyclyls containing two heteroatoms include, but are not limited to: piperazinyl, morpholinyl, dithianyl, and dioxanyl. Exemplary 6-membered heterocyclyls containing three heteroatoms include, but are not limited to: triazinanyl. Exemplary 7-membered heterocyclyls containing one heteroatom include, but are not limited to: azepanyl, oxepanyl, and thiepanyl. Exemplary 5-membered heterocyclyls each of which is fused with a C₆ aryl ring (also referred to herein as a 5,6-bicycloheterocyclyl) include, but are not limited to: dihydroindolyl, isodihydroindolyl, dihydrobenzofuryl, dihydrobenzothienyl, and benzoxazolinonyl. Exemplary 6-membered heterocyclyls each of which is fused with a C₆ aryl ring (also referred to herein as a 6,6-bicycloheterocyclyl) include, but are not limited to: tetrahydroquinolinyl, and tetrahydroisoquinolinyl. The heteocyclyl can also be substituted with one or more substitutes, for example, with 1-5 substitutes, with 1-3 substitutes, or with 1 substitute.

“C₆₋₁₀ aryl” refers to a monocyclic or polycyclic (e.g., bicyclic) group that is a 4n+2 aromatic ring system having 6 to 10 ring carbon atoms and no heteroatom (e.g., with 6 or 10 π electrons shared in a cyclic arrangement). In some embodiments, an aryl has six ring carbon atoms (“C₆ aryl”; e.g., phenyl). In some embodiments, an aryl has ten ring carbon atoms (“C₁₀ aryl”; e.g., a naphthyl, such as 1-naphthyl and 2-naphthyl). An aryl herein also includes a ring system in which an aforementioned aryl ring is fused with one or more cycloalkyls or heterocyclyls through the connection sites on said aryl ring; in this context, the number of carbon atoms still represents the number of carbon atoms in said aryl ring system. The aryl can also be substituted with one or more substitutes, for example, with 1-5 substitutes, with 1-3 substitutes, or with 1 substitute.

“5-membered to 14-membered heteroaryl” refers to a 5-membered to 14-membered monocyclic or bicyclic group of a 4n+2 aromatic ring system (e.g., with 6 or 10 π electrons shared in a cyclic arrangement) that has ring carbon atom(s) and 1 to 5 ring heteroatoms, wherein each heteroatom is independently selected from nitrogen, oxygen and sulfur. In said heteroaryl containing one or more nitrogen atoms, the connection site may be a carbon or nitrogen atom as long as the valence permits. A bicyclic heteroaryl system herein may comprise one or more heteroatoms in one or both rings thereof. A heteroaryl herein also includes a ring system in which an aforementioned heteroaryl ring is fused with one or more cycloalkyls or heterocyclyls through the connection sites on said heteroaryl ring; in this context, the number of carbon atoms still represents the number of carbon atoms in said heteroaryl ring system. In some embodiments, a 5-membered to 10-membered heteroaryl is particularly preferred, which is a 4n+2 aromatic ring system of a 5-membered to 10-membered monocyclic or bicyclic ring with ring carbon atom(s) and 1 to 4 ring heteroatoms. In some embodiments, a 5-membered to 6-membered heteroaryl is particularly preferred, which is a 4n+2 aromatic ring system of a 5-membered to 6-membered monocyclic or bicyclic ring with ring carbon atom(s) and 1 to 4 ring heteroatoms. Exemplary 5-membered heteroaryls containing one heteroatom include, but are not limited to: pyrrolyl, furyl, and thienyl. Exemplary 5-membered heteroaryls containing two heteroatoms include, but are not limited to: imidazolyl, pyrazolyl, oxazolyl, isoxazolyl, thiazolyl, and isothiazolyl. Exemplary 5-membered heteroaryls containing three heteroatoms include, but are not limited to: triazolyl, oxadiazolyl (e.g., 1,2,4-oxadiazolyl), and thiadiazolyl. Exemplary 5-membered heteroaryls containing four heteroatoms include, but are not limited to: tetrazolyl. Exemplary 6-membered heteroaryls containing one heteroatom include, but are not limited to: pyridinyl. Exemplary 6-membered heteroaryls containing two heteroatoms include, but are not limited to: pyridazinyl, pyrimidinyl, and pyrazinyl. Exemplary 6-membered heteroaryls containing three or four heteroatoms include, but are not limited to: triazinyl, and tetrazinyl. Exemplary 7-membered heteroaryls containing one heteroatom include, but are not limited to: azepinyl, oxepinyl, and thiepinyl. Exemplary 5,6-bicycloheteroaryls include, but are not limited to: indolyl, isoindolyl, indazolyl, benzotriazolyl, benzothienyl, isobenzothienyl, benzofuryl, benzoisofuryl, benzimidazolyl, benzoxazolyl, benzisoxazolyl, benzooxadiazolyl, benzothiazolyl, benzoisothiazolyl, benzothiadiazolyl, indolizinyl, and purinyl. Exemplary 6,6-bicycloheteroaryls include, but are not limited to: naphthalidinyl, pteridinyl, quinolyl, isoquinolyl, cinnolinyl, quinoxalinyl, phthalazinyl, and quinazolinyl. The heterocyclyl can also be substituted with one or more substitutes, for example, with 1-5 substitutes, with 1-3 substitutes, or with 1 substitute.

Alkyl, alkenyl, alkynyl, cycloalkyl, heterocyclyl, aryl and heteroaryl as defined herein are optionally substituted groups.

Exemplary substituents on carbon atoms include, but are not limited to: halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), alkyl, haloalkyl, alkenyl, alkynyl, cycloalkyl, heterocyclyl, aryl and heteroaryl, wherein each alkyl, alkenyl, alkynyl, cycloalkyl, heterocyclyl, aryl and heteroaryl is dependently substituted with 0, 1, 2, 3, 4 or 5 Rad groups;
or wherein two geminal hydrogens at a carbon atom are substituted by a group, such as ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb} or ═NOR^{cc};
each of R^{aa} is independently selected from alkyl, haloalkyl, alkenyl, alkynyl, cycloalkyl, heterocyclyl, aryl and heteroaryl, or two R^{aa} groups are connected each other to form a heterocyclyl ring or heteroaryl ring, wherein each alkyl, alkenyl, alkynyl, cycloalkyl, heterocyclyl, aryl and heteroaryl is dependently substituted with 0, 1, 2, 3, 4 or 5 R^{dd} groups; each of R^{bb} is independently selected from: hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{bb} groups are connected each other to form a heterocyclyl ring or heteroaryl ring, wherein each alkyl, alkenyl, alkynyl, cycloalkyl, heterocyclyl, aryl and heteroaryl group is independently substituted with 0, 1, 2, 3, 4 or 5 R^{dd} groups;
each of R^{cc} is independently selected from hydrogen, alkyl, haloalkyl, alkenyl, alkynyl, cycloalkyl, heterocyclyl, aryl and heteroaryl, or two R^{cc} groups are connected to form a heterocyclyl ring or heteroaryl ring, wherein each alkyl, alkenyl, alkynyl, cycloalkyl, heterocyclyl, aryl and heteroaryl is independently substituted with 0, 1, 2, 3, 4 or 5 R^{dd} groups;
each of R^{dd} is independently selected from: halogen, —CN, —NO₂, —N₃, —SO₂H, —SO₃H, —OH, —OR^{cc}, —ON(R^{ff})₂, —N(R^{ff})₂, —N(R)₃⁺X⁻, —N(OR^{ee})R^{ff}, —SH, —SR^{cc}, —SSR^{ee}, —C(═O)R^{ee}, —CO₂H, —CO₂R^{ee}, —OC(═O)R^{ee}, —OCO₂R^{cc}, —C(═O)N(R^{ff})₂, —OC(═O)N(R^{ff})₂, —NR^{ff}C(═O)R^{ee}, —NR^{ff}CO₂R^{ee}, —NR^{ee}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{cc}, —SO₂OR^{cc}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, alkyl, haloalkyl, alkenyl, alkynyl, cycloalkyl, heterocyclyl, aryl, heteroaryl, wherein each of alkyl, alkenyl, alkynyl, cycloalkyl, heterocyclyl, aryl and heteroaryl is independently substituted with 0, 1, 2, 3, 4 or 5 R^{gg} groups, or two geminal R^{dd} substituents may be combined to form ═O or ═S;
each of R^{cc} is independently selected from: alkyl, haloalkyl, alkenyl, alkynyl, cycloalkyl, aryl, heterocyclyl and heteroaryl, wherein each alkyl, alkenyl, alkynyl, cycloalkyl, heterocyclyl, aryl and heteroaryl is independently substituted with 0, 1, 2, 3, 4 or 5 R^{gg} groups;
each of R^{ff} is independently selected from hydrogen, alkyl, haloalkyl, alkenyl, alkynyl, cycloalkyl, heterocyclyl, aryl and heteroaryl, or two R^{f} groups are connected each other to form a heterocyclyl ring or heteroaryl ring, wherein each alkyl, alkenyl, alkynyl, cycloalkyl, heterocyclyl, aryl and heteroaryl is independently substituted with 0, 1, 2, 3, 4 or 5 R^{gg} groups;
each of R^{gg} is independently selected from: halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺x⁻, —NH(C₁₋₆ alkyl)₂⁺x⁻, —NH₂(C₁₋₆ alkyl)⁺x⁻, —NH₃⁺x⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆Haloalkyl, C₂-C₆alkenyl, C₂-C₆alkynyl, C₃-C₇Cycloalkyl, C₆-C₁₀Aryl, C₃-C₇Heterocyclyl, C₅-C₁₀Heteroaryl; or two geminal R^{gg} substituents may be connected to form ═O or ═S; wherein, X- is a counterion.

Exemplary substituents on a nitrogen atom include, but are not limited to: hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{ee})N(R^{cc})₂, —SO₂N(R^{cc})₂, —SO₂R^{cc}, —SO₂OR^{cc}, —SOR^{aa}, —C(═S)N(R′)₂, —C(═O)SR^{ee}, —C(═S)SR^{ee}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, cycloalkyl, heterocyclyl, aryl and heteroaryl, or two R^{cc} groups connecting to said nitrogen atom are connected to form a heterocyclyl ring or heteroaryl ring, wherein each alkyl, alkenyl, alkynyl, cycloalkyl, heterocyclyl, aryl and heteroaryl is independently substituted with 0, 1, 2, 3, 4 or 5 R^{dd} groups, and wherein Ra, R^{bb}, R^{cc} and R^{dd} are as described above.

### Some Additional Definitions

The term “siRNA” herein is a class of dsRNA molecules which can mediate the silencing of target RNA (e.g., mRNA, e.g., transcript of a gene encoding a protein) complementary thereto. A siRNA is generally double-stranded, including an antisense strand complementary to the target RNA thereof and a sense strand complementary to this antisense strand. For the sake of convenience, such an mRNA is also referred to herein as mRNA to be silenced, and such a gene is also called target gene. Usually, an RNA to be silenced herein is an endogenous gene or a pathogen gene. In addition, RNAs other than mRNA (e.g., tRNA) as well as viral RNA may also be targeted.

The term “antisense strand” herein refers to a strand of a siRNA, wherein said strand contains a region that is completely, sufficiently or substantially complementary to the target sequence thereof. The term “sense strand” herein refers to a strand of a siRNA, wherein said strand contains a region that completely, sufficiently or substantially complementary to a region of an antisense strand as defined herein.

The term “complementary region” herein refers to a region on an antisense strand that is completely, sufficiently or substantially complementary to the target mRNA sequence thereof. In cases where a complementary region is incompletely complementary to the target sequence thereof, a mismatch may be located in an internal or terminal region of the molecule. Typically, a mismatch most tolerant is located in a terminal region, e.g., within 5, 4, 3, 2 or 1 nucleotide at the 5′ and/or 3′ end. A region in an antisense strand, which is most sensitive to mismatch, is called “seed region”. For example, in a siRNA containing a strand of 19 nt, the 19^{th} site (counting from the 5′ end to the 3′ end) can tolerate some mismatches.

The term “complementary” refers to the ability of a first polynucleotide to hybridize with a second polynucleotide under certain conditions, such as stringent conditions. For example, stringent conditions may include 400 mM NaCl, 40 mM PIPES pH 6.4, 1 mM EDTA, and 50° C. or 70° C. for 12-16 hours. With respect to fulfilling the above required capabilities related to the hybridization ability thereof, said “complementary” sequences may also include or be entirely composed of non-Watson-Crick base pairs and/or base pairs formed from non-natural as well as modified nucleotides. Such non-Watson-Crick base pairs include, but are not limited to, G:U wobble base pairing or Hoogsteen base pairing.

A polynucleotide that is “at least partially complementary”, “sufficiently complementary” or “substantially complementary” to a messenger RNA (mRNA) refers to a polynucleotide that is substantially complementary to a continuous portion of the mRNA of interest. For example, a polynucleotide is at least partially complementary to an mRNA encoding PCSK9, when the sequence thereof is substantially complementary to an uninterrupted portion of said PCSK9 mRNA. The terms “complementary,” “completely complementary,” “sufficiently complementary” and “substantially complementary” as used herein may be applied to base pairing between the sense strand and antisense strand of a siRNA, or between the antisense strand of a siRNA reagent and the target sequence thereof.

“Sufficiently complementary” refers to the extent to which the sense strand only needs to be complementary to the antisense strand to maintain the overall double-stranded character of the molecule. In other words, although perfect complementarity is generally desired, in some cases, particularly in the antisense strand, one or more, e.g., 6, 5, 4, 3, 2, or 1 mismatch (relative to the target mRNA) may be included, but the sense and antisense strands can still maintain the overall double-stranded character of the molecule.

The term “shRNA” herein refers to short hairpin RNA. An shRNA comprises two short inverted repeat sequences. An shRNA cloned into an shRNA expression vector comprises two short inverted repeat sequences, separated by a loop sequence, forming a hairpin structure and controlled by the RNA polymerase III (pol III) promoter. Subsequently, 5 to 6 Ts are ligated as transcription terminators of pol III.

“Nucleoside” is a compound comprising two substances: one is a purine base or a pyrimidine base, and the other is a ribose or a deoxyribose. “Nucleotide” is a compound comprising three substances: one is a purine base or a pyrimidine base, another is a ribose or deoxyribose, and the third is a phosphoric acid. “Oligonucleotide” is a nucleic acid molecule (RNA or DNA) having a length of less than 100, 200, 300 or 400 nucleotides.

“Ribose” is a pentose aldose, consisting of five carbon atoms, wherein the carbon atom number adjacent to the oxygen atom is 1′ (for short 1′ carbon or 1′ end). The carbon atoms are numbered in a clockwise order consecutively, linking to the base at 1′ position and the phosphate group at 5′ position. The structure and carbon atom number are shown as follows.

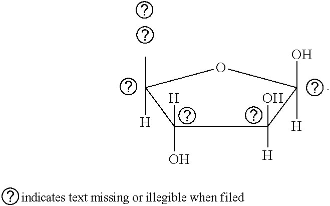

The term “base” is a fundamental building block of nucleosides, nucleotides and nucleic acids; as always containing nitrogen, said base is also referred to as “nitrogenous base.” Unless otherwise specified, the capital letters herein, i.e., A, U, T, G and C, denote the bases of nucleotides, which is adenine, uracil, thymine, guanine and cytosine, respectively.

As used herein, the “modification” of nucleotides includes, but is not limited to: methoxyl substitution (methoxy-modified), fluorine substitution (fluoro-modified), connection with a phosphorothioate group, or protection with a conventional protecting group. For example, a fluoro-modified nucleotide refers to a nucleotide formed by substituting the hydroxyl at the 2′ position of the ribosyl of the nucleotide with a fluorine atom, while a methoxy-modified nucleotide refers to a nucleotide formed by substituting the 2-hydroxyl of the ribosyl with a methoxyl.

“Modified nucleotides” herein include, but are not limited to: a 2-O-methyl modified nucleotide, a 2′-fluoro modified nucleotide, a 2′-deoxy-modified nucleotide, an inosine ribonucleotide, an abasic nucleotide, an inverted abasic deoxyribonucleotide, a nucleotide comprising a phosphorothioate group, a nucleotide modified by vinylphosphonate, a locked nucleotide, 2′-amino-modified nucleotide, 2′-alkyl-modified nucleotide, a morpholino nucleotide, a phosphoramidate, a non-natural base comprising nucleotide, and a terminal nucleotide linked to a cholesteryl derivative or a dodecanoic acid bisdecylamide group, deoxyribonucleotide, or a nucleotide with protection of a conventional protecting group. For example, a 2′-fluoro modified nucleotide refers to a nucleotide formed by substituting the hydroxyl at the 2′ position of the ribosyl in a nucleotide with a fluorine atom. Said 2′-deoxy-modified nucleotide refers to a nucleotide formed by substituting the 2-hydroxyl of the ribosyl with a methoxyl.

“Protecting group” refers to any atom or group of atoms added to a molecule to prevent undesired chemical reactions of existing groups within the molecule. A “protecting group” may be an unstable chemical moiety known in the art, which is used to protect reactive groups such as hydroxyl, amino and thiol groups to prevent undesired or premature reactions during chemical synthesis. Protecting groups are typically used selectively and/or orthogonally to protect sites during the reactions of other reactive sites, which can then be removed to leave the unprotected groups intact or available for further reactions.

A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; a substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl)ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr or DMT); 4,4′,4″-trimethoxytrityl (TMTr); and those described herein). Preferred protecting groups are selected from acetyl (Ac), benzoyl (Bzl), benzyl (Bn), isobutyryl (iBu), phenylacetyl, benzyloxymethyl acetal (BOM), beta-methoxyethoxymethyl ether (MEM), methoxymethylether (MOM), p-methoxybenzyl ether (PMB), methylthiomethyl ether, pivaloyl (Piv), tetrahydropyranyl (THP), triphenylmethyl (Trt), methoxytrityl [(4-methoxyohenyl)diphenylmethyl-](MMT), dimethoxytrityl, [bis-(4-methoxyphenyl)phenylmethyl (DMT), trimethylsilyl ether (TMS), tert-butyldimethylsilyl ether (TBDMS), tri-iso-propylsilyloxymethyl ether (TOM), tri-isopropylsilyl ether (TIPS), methyl ethers, ethoxyethyl ethers (EE) N,N-dimethylformamidine and 2-cynaonethyl (CE).

“Hydroxy-protecting group” refers to a group that can prevent a hydroxyl from undergoing chemical reactions and can be removed under specific conditions to restore the hydroxyl. The main hydroxy-protecting groups include silane-type, acyl-type or ether-type protecting groups, preferably the following:
trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), p-methoxybenzyloxymethyl (PMBM), —C(O)CH₂CH₂C(O)OH or 4,4′-dimethoxytrityl, preferably —C(O)CH₂CH₂C(O)OH or 4,4′-dimethoxytrityl, more preferably —C(O)CH₂CH₂C(O)OH.

As used herein, the term “pharmaceutically acceptable salt” represents carboxylates or amino acid salts of a compound of the present invention, which are suitable for contact with patient tissues within the scope of sound medical judgment without causing excessive toxicity, irritation, allergic reactions, etc., and are effective in terms of the intended use with a reasonable benefit/risk ratio; said salt includes, where applicable, the zwitterionic form of a compound of the present invention.

The present invention includes tautomers, which are functional-group isomers resulting from the rapid migration of an atom in a molecule between two positions. A compound with different tautomeric forms, said herein, refers to all the tautomers and does not be restricted to any specific tautomeric form.

A compound of the present invention may include one or more asymmetric centers, and thus may exist in various stereoisomeric forms, such as enantiomers and/or diastereomers. For example, a compound of the present invention may be one of the forms of enantiomer, diastereoisomer or geometric isomer (e.g. a cis isomer or a trans isomer), or may be a mixture of any type of stereoisomerism, including a racemic mixture and a mixture enriched with one or more forms of stereoisomer. An isomer herein may be achieved by separating from a mixture via any method known to those skilled in the art, wherein the method includes chiral high-pressure liquid chromatography (HPLC) and the formation and crystallization of chiral salts; alternatively, a preferred isomer may be prepared through asymmetric synthesis.

The present invention also includes isotopically labeled compounds (isotopic variants) which are equivalent to those described by formula (I), except that one or more atoms are replaced with atoms with an atomic mass or mass number different from that common in nature. Examples of isotopes which may be incorporated into a compound of the present invention include isotopes of hydrogen, carbon, nitrogen, oxygen, phosphorus, sulfur, fluorine, and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl. Any compounds based on a compound of the present invention and containing any aforementioned isotope and/or any isotope of other atoms, the prodrugs thereof and the pharmaceutically acceptable salts of said compounds or said prodrugs all fall in the scope of the present invention. Certain isotopically labeled compounds of the present invention, such as a compound into which any radioisotope (e.g., ³H and ¹⁴C) is introduced, may be used for distribution determinations of a drug and/or the substrate tissue thereof. Tritium, i.e. ³H and carbon-14, i.e. ¹⁴C isotopes are particularly preferred, because they can be easily prepared and detected. Furthermore, substitution with an isotope heavier, such as deuterium, i.e. ²H, may in some cases be preferred because resultant increased metabolic stability may provide therapeutic benefits such as prolonged in vivo half-life or reduced dosage. An isotopically labeled compound of formula (I) of the present invention and the prodrug thereof may generally be prepared with any readily available isotopically labeled reagent instead of any non-isotopically labeled reagent, in a procedure described below and/or in a process disclosed in any of the examples and preparations.

### The Compounds of the Present Invention

The present invention specifically relates to a compound of formula (I), or a pharmaceutically acceptable salt, tautomer or stereoisomer thereof.

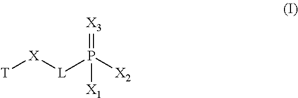

wherein,
X₁ is selected from OR, OP₁, SP₁ or NR_{b}R_{c};
Rₐ is selected from H, C₁₋₆ alkyl, C₁₋₆ haloalkyl, C₂₋₆ alkenyl or C₂₋₆ alkynyl, which is optionally deuterated or fully deuterated;
R_{b} and R_{c} are independently selected from H, C₁₋₆ alkyl or C₁₋₆ haloalkyl; R_{b} and R_{c} may optionally be substituted with D, C₆₋₁₀ aryl or 5-10-membered heteroaryl, or fully deuterated;
X₂ is OH, OP₁ or SP₁, or X₂ is a nucleoside moiety, which connects to P via a hydroxyl or a sulfydryl on the 2′, 3′ or 5′ carbon of the ribose;
X₃ is independently selected from O or S;
T is selected from

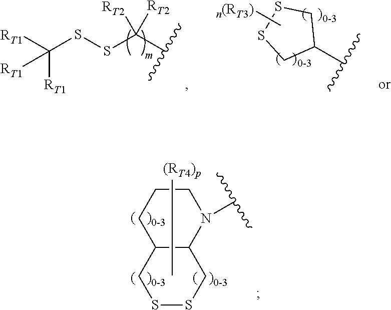

each R_{T1} is independently selected from H, D, halogen, CN, C₁₋₆ alkyl, C₁₋₆ haloalkyl, C₁₋₆ haloalkyl, C₂₋₆ alkenyl, C₂₋₆ alkynyl or a GalNAc-containing chain, which is optionally deuterated or fully deuterated;
each R_{T2} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
each R_{T3} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
each R_{T4} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
m is 0, 1, 2, 3, 4 or 5;
n is 0, 1, 2, 3, 4 or 5;
p is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, halogen, C₁₋₆ alkyl or C₁₋₆ haloalkyl, which is optionally deuterated or fully deuterated;
L is —Ar—(CH₂)₁₋₆—O—, wherein each CH₂ may optionally be substituted with R #; R # is selected from H, D, halogen, CN, C₁₋₆ alkyl, C₁₋₆ haloalkyl, C₂₋₆ alkenyl or C₂₋₆ alkynyl, which is optionally deuterated or fully deuterated;
Ar in L is linked to X; the oxygen atom in L is linked to the phosphorus atom;
Ar is selected from C₃₋₁₀ cycloalkyl, 3-10-membered heterocyclyl, C₆₋₁₀ aryl or 5-14 membered heteroaryl; C₃₋₁₀ cycloalkyl, 3-10-membered heterocyclyl, C₆₋₁₀ aryl or 5-10-membered heteroaryl may optionally be substituted with 1, 2, 3, 4 or 5 R*;
R* is selected from H, D, halogen, CN, C₁₋₆ alkyl, C₁₋₆ haloalkyl, C₂₋₆ alkenyl or C₂₋₆ alkynyl, which is optionally deuterated or fully deuterated;
wherein P₁ is selected from a protecting group, preferably a hydroxyl protecting group, e.g., trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), p-methoxybenzyloxymethyl (PMBM), —C(O)CH₂CH₂C(O)OH or 4,4′-dimethoxytrityl, preferably —C(O)CH₂CH₂C(O)OH or 4,4′-dimethoxytrityl, more preferably —C(O)CH₂CH₂C(O)OH.

The present invention also relates to an oligonucleotide, comprising one, two or more compounds of formula (Ia), or a pharmaceutically acceptable salt, tautomer or stereoisomer thereof; The present invention also relates to a dsRNA having a sense strand and an antisense strand, wherein each strand has 14-30 nucleotides; wherein the antisense strand comprise a sequence sufficiently complementary to the sense strand and target mRNA; wherein the sense stand and/or the antisense strand comprise one or more compounds of formula (Ia), or a pharmaceutically acceptable salt, tautomer or stereoisomer thereof:

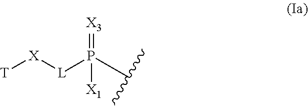

wherein,
[symbol] is selected from ORₐ or NR_{b}R_{c}, or represents a bond connecting to a hydroxyl or a sulfydryl of the 5′ carbon of the ribose of an adjacent nucleotide;
X₁ is selected from ORₐ or NR_{b}R_{c}, or is a bond connecting to a hydroxyl or a sulfydryl of the 2′ or 3′ carbon of the ribose of the other adjacent nucleotide;
and [symbol] and X₁ are not ORₐ or NR_{b}R_{c} simultaneously;
Rₐ is selected from H, C₁₋₆ alkyl, C₁₋₆ haloalkyl, C₂₋₆ alkenyl or C₂₋₆ alkynyl, which is optionally deuterated or fully deuterated;
R_{b} and R_{c} is independently selected from H, C₁₋₆ alkyl or C₁₋₆ haloalkyl; R_{b} and R_{c} may optionally be substituted with D, C₆₋₁₀ aryl or 5-10-membered heteroaryl, or fully deuterated;
X₃ is independently selected from O or S;
T is selected from

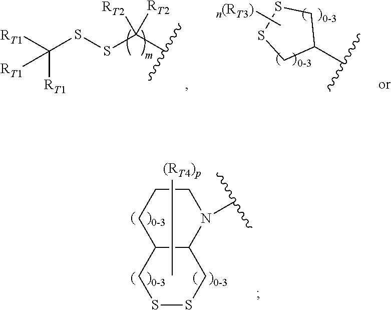

each R_{T1} is independently selected from H, D, halogen, CN, C₁₋₆ alkyl, C₁₋₆ haloalkyl, C₁₋₆ haloalkyl, C₂₋₆ alkenyl, C₂₋₆ alkynyl or a a GalNAc-containing chain, which is optionally deuterated or fully deuterated;
each R_{T2} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
each R_{T3} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
each R_{T4} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
m is 0, 1, 2, 3, 4 or 5;
n is 0, 1, 2, 3, 4 or 5;
p is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, halogen, C₁₋₆ alkyl or C₁₋₆ haloalkyl, which is optionally deuterated or fully deuterated;
L is —Ar—(CH₂)₁₋₆—O—, wherein each CH₂ may optionally be substituted with R #; R # is selected from H, D, halogen, CN, C₁₋₆ alkyl, C₁₋₆ haloalkyl, C₂₋₆ alkenyl or C₂₋₆ alkynyl, which is optionally deuterated or fully deuterated;
Ar in L is linked to X; the oxygen atom in L is linked to the phosphorus atom;
Ar is selected from C₃₋₁₀ cycloalkyl, 3-10-membered heterocyclyl, C₆₋₁₀ aryl or 5-14-membered heteroaryl; C₃₋₁₀ cycloalkyl, 3-10-membered heterocyclyl, C₆₋₁₀ aryl or 5-10-membered heteroaryl may optionally be substituted with 1, 2, 3, 4 or 5 R*;
R* is selected from H, D, halogen, CN, C₁₋₆ alkyl, C₁₋₆ haloalkyl, C₂₋₆ alkenyl or C₂₋₆ alkynyl, which is optionally deuterated or fully deuterated.

The present invention also related to an oligonucleotide comprising a compound of formula (II), or a pharmaceutically acceptable salt, tautomer or stereoisomer thereof:

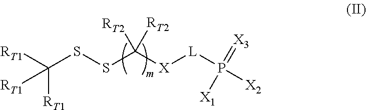

wherein,
X₁ is selected from ORₐ or NR_{b}R_{c};
Rₐ is selected from H, C₁₋₆ alkyl or C₁₋₆ haloalkyl, which is optionally deuterated or fully deuterated;
R_{b} and R_{c} is selected from H, C₁₋₆ alkyl or C₁₋₆ haloalkyl; R_{b} and R_{c} may optionally be substituted with D, C₆₋₁₀ aryl or 5-10-membered heteroaryl, or fully deuterated;
X2 is the remainder of the oligonucleotide, which connects to P via a hydroxyl or a sulfydryl on the 2′, 3′ or 5′ carbon of the ribose of its first nucleotide counting from the 5′ end;
X₃ is independently selected from O or S;
each R_{T1} is independently selected from H, D, C₁₋₆ alkyl, C₁₋₆ haloalkyl, C₂₋₆ alkenyl, C₂₋₆ alkynyl or a a GalNAc-containing chain, which is optionally deuterated or fully deuterated;
each R_{T2} is independently selected from H, D, C₁₋₆ alkyl, C₁₋₆ haloalkyl, C₂₋₆ alkenyl or C₂₋₆ alkynyl, which is optionally deuterated or fully deuterated;
m is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₆ alkyl or C₁₋₆ haloalkyl, which is optionally deuterated or fully deuterated;
L is —Ar—(CH₂)₁₋₆—O—, wherein each CH₂ may optionally be substituted with 1 or 2 R #; R # is selected from H, D, C₁₋₆ alkyl or C₁₋₆ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl is optionally substituted by 1, 2, 3, 4 or 5 R*;
R* is selected from C₁₋₆ alkyl, C₁₋₆ haloalkyl, C₂₋₆ alkenyl or C₂₋₆ alkynyl, which is optionally deuterated or fully deuterated.

The present invention is also directed to an oligonucleotide comprising one, two or more compounds of formula (Ia), or a pharmaceutically acceptable salt, tautomer or stereoisomer thereof:

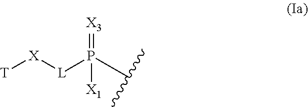

wherein,
[symbol] is selected from ORₐ or NR_{b}R_{c}, or represents a bond connecting to a hydroxyl or a sulfydryl of the 5′ carbon of the ribose of an adjacent nucleotide;
X₁ is a bond connecting to a hydroxyl or a sulfydryl of the 2′ or 3′ carbon of the ribose of the other adjacent nucleotide;
Rₐ is selected from H, C₁₋₆ alkyl, C₁₋₆ haloalkyl, C₂₋₆ alkenyl or C₂₋₆ alkynyl, which is optionally deuterated or fully deuterated;
R_{b} and R_{c} are independently selected from H, C₁₋₆ alkyl or C₁₋₆ haloalkyl; R_{b} and R_{c} may optionally be substituted with D, C₆₋₁₀ aryl or 5-10-membered heteroaryl, or fully deuterated;
X₃ is independently selected from O or S;
T is

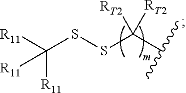

each R_{T1} is independently selected from H, D, halogen, CN, C₁₋₆ alkyl, C₁₋₆ haloalkyl, C₁₋₆ haloalkyl, C₂₋₆ alkenyl, C₂₋₆ alkynyl or a a GalNAc-containing chain, which is optionally deuterated or fully deuterated;
each R_{T2} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
m is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, halogen, C₁₋₆ alkyl or C₁₋₆ haloalkyl, which is optionally deuterated or fully deuterated;
L is —Ar—(CH₂)₁₋₆—O—, wherein each CH₂ may optionally be substituted with R #; R # is selected from H, D, halogen, CN, C₁₋₆ alkyl, C₁₋₆ haloalkyl, C₂₋₆ alkenyl or C₂₋₆ alkynyl, which is optionally deuterated or fully deuterated;
Ar in L is linked to X; the oxygen atom in L is linked to the phosphorus atom;
Ar is selected from C₃₋₁₀ cycloalkyl, 3-10-membered heterocyclyl, C₆₋₁₀ aryl or 5-14-membered heteroaryl; C₃₋₁₀ cycloalkyl, 3-10-membered heterocyclyl, C₆₋₁₀ aryl or 5-10-membered heteroaryl may optionally be substituted with 1, 2, 3, 4 or 5 R*;
R* is selected from H, D, halogen, CN, C₁₋₆ alkyl, C₁₋₆ haloalkyl, C₂₋₆ alkenyl or C₂₋₆ alkynyl, which is optionally deuterated or fully deuterated.
X₁, X₂, X₃ and

[symbol]

In one embodiment, X₁ is ORₐ. In another embodiment, X₁ is OP₁. In another embodiment, X₁ is SP₁. In another embodiment, X₁ is NR_{b}R_{c}. In another embodiment, X₁ is a bond connecting to a hydroxyl or a sulfydryl of the 5′ carbon of the ribose of the other adjacent nucleotide. In another embodiment, X₁ is a bond connecting to a hydroxyl or a sulfydryl of the 2′ carbon of the ribose of the other adjacent nucleotide. In another embodiment, X₁ is bond connecting to a hydroxyl or a sulfydryl of the 5′ carbon of the ribose of the other adjacent nucleotide.

In one embodiment, X₁ is a bond connecting to a hydroxyl or a sulfydryl of the 2′ or 3′ carbon of the ribose of the other adjacent nucleotide.

In a more specific embodiment, X₁ is OH. In another more specific embodiment, X₁ is

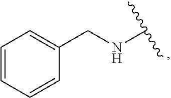 [SMILES: *NCc1ccccc1]

In one embodiment, X₂ is OH. In another embodiment, X₂ is OP₁. In another embodiment, X₂ is SP₁. In another embodiment, X₂ is a nucleoside moiety, which connects to P via a hydroxyl or a sulfydryl on the 2′ carbon of the ribose. In another embodiment, X₂ is a nucleoside moiety, which connects to P via a hydroxyl or a sulfydryl on the 3′ carbon of the ribose. In another embodiment, X₂ is a nucleoside moiety, which connects to P via a hydroxyl or a sulfydryl on the 5′ carbon of the ribose.

In one embodiment, X₂ is the remainder of the oligonucleotide, which connects to P via a hydroxyl or a sulfydryl on the 2′, 3′ or 5′ carbon of the ribose of its first nucleotide counting from the 5′ end. For example X₂ connects to P via a hydroxyl or a sulfydryl on the 5′ carbon of the ribose of its first nucleotide counting from the 5′ end.

In one embodiment, X₃ is O. In another embodiment, X₃ is S.

In one embodiment, [symbol] is ORₐ. In another embodiment, [symbol] is NR_{b}R_{c}. In another embodiment, [symbol] is a bond connecting to a hydroxyl or a sulfydryl of the 2′ or 3′ carbon of the ribose of the other adjacent nucleotide. In another embodiment, [symbol] and X₁ are not ORₐ or NR_{b}R_{c} simultaneously.

In one embodiment, [symbol] represents a bond connecting to a hydroxyl or a sulfydryl of the 5′ carbon of the ribose of an adjacent nucleotide.

### T

In one embodiment, T is

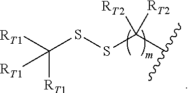

In another embodiment, T is

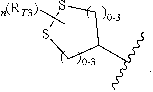

In another embodiment, T is

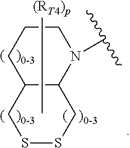

In another embodiment, T is

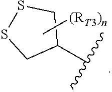

In another embodiment, T is

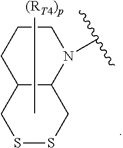

In one embodiment, T is

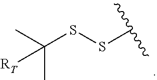

In another embodiment, T is

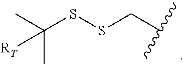

In another embodiment, T is

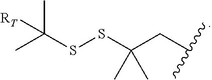

In a more specific embodiment, T is

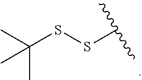 [SMILES: *SSC(C)(C)C]

In another more specific embodiment, T is

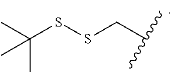 [SMILES: *CSSC(C)(C)C]

In another more specific embodiment, T is

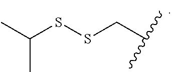 [SMILES: *CSSC(C)C]

In another more specific embodiment, T is

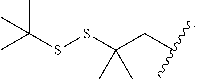 [SMILES: *CC(C)(C)SSC(C)(C)C]

In another more specific embodiment, T is

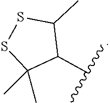 [SMILES: *C1C(C)SSC1(C)C]

In another more specific embodiment, T is

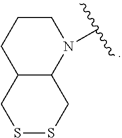 [SMILES: *N1CCCC2CSSCC21]

R_{T1}, R_{T2}, R_{T3}, R_{T4}, R_{T}, m, n and p

In one embodiment, R_{T1} is H. In another embodiment, R_{T1} is D. In another embodiment, R_{T1} is halogen. In another embodiment, R_{T1} is CN. In another embodiment, R_{T1} is C₁₋₆ alkyl, for example C₁₋₄ alkyl. In another embodiment, R_{T1} is C₁₋₆ haloalkyl, for example C₁₋₄ haloalkyl. In another embodiment, R_{T1} is C₁₋₆ haloalkyl. In another embodiment, R_{T1} is C₂₋₆ alkenyl. In another embodiment, R_{T1} is C₂₋₆ alkynyl. In another embodiment, R_{T1} is a GalNAc-containing chain. In another embodiment, R_{T1} is optionally deuterated or fully deuterated.

In one embodiment, R_{T2} is H. In another embodiment, R_{T2} is D. In another embodiment, R_{T2} is halogen. In another embodiment, R_{T2} is CN. In another embodiment, R_{T2} is C₁₋₆ alkyl, for example C₁₋₄ alkyl. In another embodiment, R_{T2} is C₁₋₆ haloalkyl, for example C₁₋₄ haloalkyl. In another embodiment, R_{T2} is C₂₋₆ alkenyl. In another embodiment, R_{T2} is C₂₋₆ alkynyl. In another embodiment, R_{T2} is optionally deuterated or fully deuterated.

In one embodiment, R_{T3} is H. In another embodiment, R_{T3} is D. In another embodiment, R_{T3} is halogen. In another embodiment, R_{T3} is CN. In another embodiment, R_{T3} is C₁₋₆ alkyl, for example C₁₋₄ alkyl. In another embodiment, R_{T3} is C₁₋₆ haloalkyl, for example C₁₋₄ haloalkyl. In another embodiment, R_{T3} is C₂₋₆ alkenyl. In another embodiment, R_{T3} is C₂₋₆ alkynyl. In another embodiment, R_{T3} is optionally deuterated or fully deuterated.

In one embodiment, R_{T4} is H. In another embodiment, R_{T4} is D. In another embodiment, R_{T4} is halogen. In another embodiment, R_{T4} is CN. In another embodiment, R_{T4} is C₁₋₆ alkyl, for example C₁₋₄ alkyl. In another embodiment, R_{T4} is C₁₋₆ haloalkyl, for example C₁₋₄ haloalkyl. In another embodiment, R_{T4} is C₂₋₆ alkenyl. In another embodiment, R_{T4} is C₂₋₆ alkynyl. In another embodiment, R_{T4} is optionally deuterated or fully deuterated.

In one embodiment, R_{T} is H. In another embodiment, R_{T} is D. In another embodiment, R_{T} is CH₃. In another embodiment, R_{T} is a a GalNAc-containing chain. In another embodiment, R_{T} is optionally deuterated or fully deuterated.

In one embodiment, m is 0. In another embodiment, m is 1. In another embodiment, m is 2.

In another embodiment, m is 3. In another embodiment, m is 4. In another embodiment, m is 5.

In one embodiment, n is 0. In another embodiment, n is 1. In another embodiment, n is 2.

In another embodiment, n is 3. In another embodiment, n is 4. In another embodiment, n is 5.

In one embodiment, p is 0. In another embodiment, p is 1. In another embodiment, p is 2.

In another embodiment, p is 3. In another embodiment, p is 4. In another embodiment, p is 5.

### X

In one embodiment, X is bond. In another embodiment, X is —O—. In another embodiment, X is —S—. In another embodiment, X is —C(O)—. In another embodiment, X is —C(O)O—. In another embodiment, X is —OC(O)—. In another embodiment, X is —OC(O)NR_{X1}—. In another embodiment, X is —NR_{X1}C(O)O—. In another embodiment, X is —NR_{X1}C(O)—. In another embodiment, X is —C(O)NR_{X1}—.

In a more specific embodiment, X is bond. In another more specific embodiment, X is —O—. In another more specific embodiment, X is —NHC(O)O—. In another more specific embodiment, X is —OC(O)NH—. In another more specific embodiment, X is —N(CH₃)C(O)O—. In another more specific embodiment, X is —C(O)O—.

### L

In one embodiment, L is —Ar—(CH₂)₁₋₆—O—, wherein each CH₂ may optionally be substituted with R #, and wherein Ar in L is linked to X, and the oxygen atom in L is linked to the phosphorus atom. In another embodiment, L is —Ar—(CH₂)₁₋₄—O—, wherein each CH₂ may optionally be substituted with 1 or 2 R #, and wherein Ar in L is linked to X, and the oxygen atom in L is linked to the phosphorus atom.

In one embodiment, Ar is C₃₋₁₀ cycloalkyl. In another embodiment, Ar is 3-10-membered heterocyclyl. In another embodiment, Ar is C₆₋₁₀ aryl. In another embodiment, Ar is 5-14-membered heteroaryl, for example 5-10-membered heteroaryl, for example phenyl. In another embodiment, Ar may optionally be substituted with 1, 2, 3, 4 or 5 R*.

In a more specific embodiment, L is

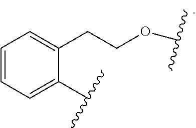 [SMILES: *OCCc1ccccc1*]

In another more specific embodiment, L is

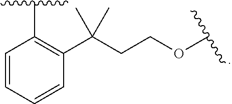 [SMILES: *OCCC(C)(C)c1ccccc1*]

In another more specific embodiment, L is

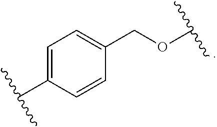 [SMILES: *OCc1ccc(*)cc1]

In another more specific embodiment, L is

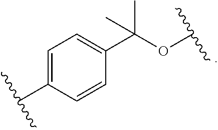 [SMILES: *OC(C)(C)c1ccc(*)cc1]

In another more specific embodiment, L is

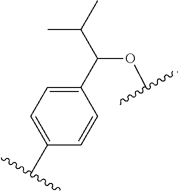 [SMILES: *OC(c1ccc(*)cc1)C(C)C ; "."]

In another more specific embodiment, L is

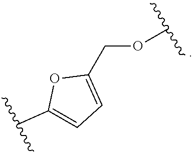 [SMILES: *OCc1ccc(*)o1]

In another more specific embodiment L is

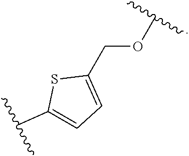 [SMILES: *OCc1ccc(*)s1]

In another more specific embodiment, L is

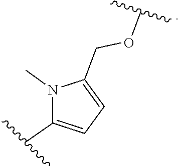 [SMILES: *OCc1ccc(*)n1C]

Rₐ, R_{b} and R_{c}

In one embodiment, Rₐ is H. In another embodiment, it is C₁₋₆ alkyl, for example C₁₋₄ alkyl. In another embodiment, it is C₁₋₆ haloalkyl, for example C₁₋₄ haloalkyl. In another embodiment, Rₐ is C₂₋₆ alkenyl. In another embodiment, Rₐ is C₂₋₆ alkynyl. In another embodiment, Rₐ is optionally deuterated or fully deuterated.

In one embodiment, R_{b} is H. In another embodiment, R_{b} is C₁₋₆ alkyl, for example C₁₋₄ alkyl. In another embodiment, R_{b} is C₁₋₆ haloalkyl, for example C₁₋₄ haloalkyl. In another embodiment, R_{b} may optionally be substituted with D, C₆₋₁₀ aryl or 5-10-membered heteroaryl, or fully deuterated.

In one embodiment, Rₑ is H. In another embodiment, Rₑ is C₁₋₆ alkyl, for example C₁₋₄ alkyl. In another embodiment, Rₑ is C₁₋₆ haloalkyl, for example C₁₋₄ haloalkyl. In another embodiment, Rₑ may optionally be substituted with D, C₆₋₁₀ aryl or 5-10-membered heteroaryl, or fully deuterated.

### R_{X1}

In one embodiment, R_{X1} is H. In another embodiment, R_{X1} is C₁₋₆ alkyl, for example C₁₋₄ alkyl. In another embodiment, R_{X1} is C₁₋₆ haloalkyl, for example C₁₋₄ haloalkyl. In another embodiment, R_{X1} is optionally deuterated or fully deuterated.

### R #

In one embodiment, R # is H. In another embodiment, R # is D. In another embodiment, R # is halogen. In another embodiment, R # is CN. In another embodiment, R # is C₁₋₆ alkyl, for example C₁₋₄ alkyl. In another embodiment, R # is C₁₋₆ haloalkyl, for example C₁₋₄ haloalkyl. In another embodiment, R # is C₂₋₆ alkenyl. In another embodiment, R # is C₂₋₆ alkynyl. In another embodiment, R # is optionally deuterated or fully deuterated.

### R*

In one embodiment, R* is H. In another embodiment, R* is D. In another embodiment, R* is halogen. In another embodiment, R* is CN. In another embodiment, R* is C₁₋₆ alkyl, for example C₁₋₄ alkyl. In another embodiment, R* is C₁₋₆ haloalkyl, for example C₁₋₄ haloalkyl. In another embodiment, R* is C₂₋₆ alkenyl. In another embodiment, R* is C₂₋₆ alkynyl. In another embodiment, R* is optionally deuterated or fully deuterated.

### PG and P₁

In one embodiment, PG is a protecting group. In another embodiment, PG is a hydroxyl protecting group, e.g., trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), p-methoxybenzyloxymethyl (PMBM), —C(O)CH₂CH₂C(O)OH or 4,4′-dimethoxytrityl, preferably —C(O)CH₂CH₂C(O)OH or 4,4′-dimethoxytrityl, more preferably —C(O)CH₂CH₂C(O)OH.

In one embodiment, P₁ is a protecting group. In another embodiment, P₁ is a hydroxyl protecting group, e.g., trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), p-methoxybenzyloxymethyl (PMBM), —C(O)CH₂CH₂C(O)OH or 4,4′-dimethoxytrityl, preferably —C(O)CH₂CH₂C(O)OH or 4,4′-dimethoxytrityl, more preferably —C(O)CH₂CH₂C(O)OH.

### GalNAc-Containing Chain

In one embodiment, the GalNAc-containing chain comprises a conjugated group of formula (X′):

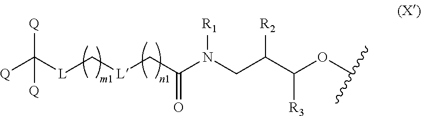

wherein,
[symbol] represents the position of attachment to biomolecules;
Q is independently H,

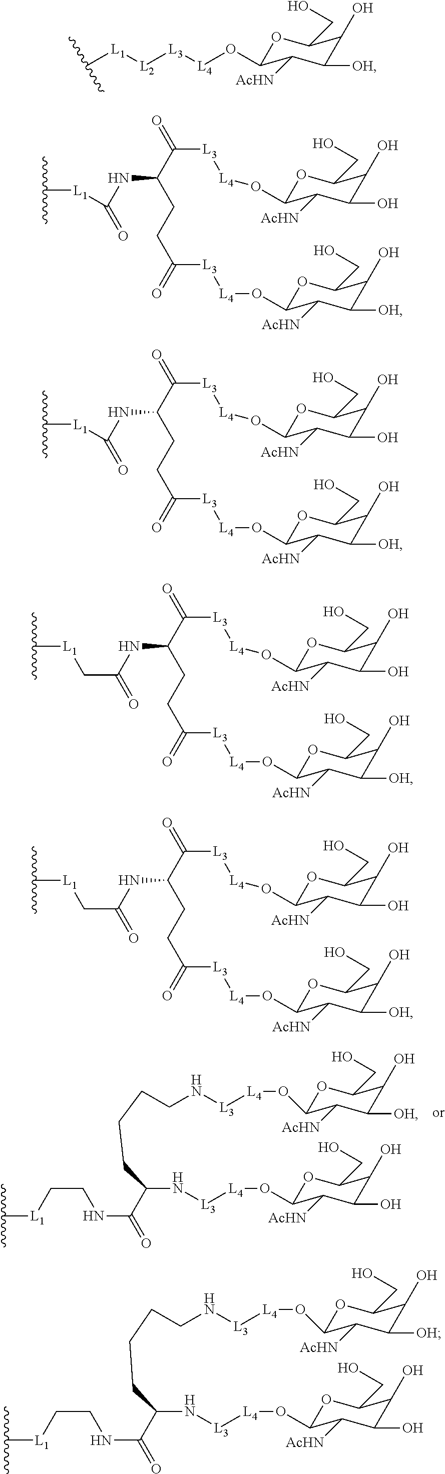

wherein L₁ is bond, —CH₂—, —CH₂CH₂—, —C(O)—, —CH₂O—, —CH₂O—CH₂CH₂O— or —NHC(O)—(CH₂NHC(O))ₐ—;
L₂ is bond or —CH₂CH₂C(O)—;
L₃ is bond, —(NHCH₂CH₂)_{b}—, —(NHCH₂CH₂CH₂)_{b}— or —C(O)CH₂—;
L₄ is —(OCH₂CH₂)ₑ—, —(OCH₂CH₂CH₂)ₑ—, —(OCH₂CH₂CH₂CH₂)ₑ—, —(OCH₂CH₂CH₂CH₂CH₂)ₑ— or —NHC(O)—(CH₂)ₐ—;
   wherein a=0, 1, 2 or 3;
   b=1, 2, 3, 4 or 5;
   c=1, 2, 3, 4 or 5;
   d=1, 2, 3, 4, 5, 6, 7 or 8;
A is bond, —CH₂O— or —NHC(O)—;
A′ is bond, —C(O)NH—, —NHC(O)— or —O(CH₂CH₂O)ₑ—;
   wherein e is 1, 2, 3, 4 or 5;
B is bond, —CH₂—, —C(O)—, -M-, —CH₂-M- or —C(O)-M-;
   wherein M is

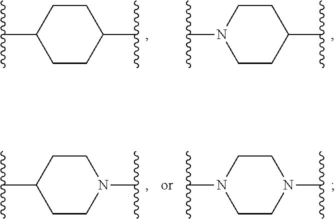 [SMILES: *C1CCC(*)CC1 ; *C1CCN(*)CC1 ; *C1CCN(*)CC1 ; *N1CCN(*)CC1]

R₁ and R₂ together form —CH₂CH₂O— or —CH₂CH(R)—O—; and R₃ is H;
or R₁ and R₃ together form —C₁₋₂ alkylene-; and R₂ is H;
   wherein R is —OR′, —CH₂OR′ or —CH₂CH₂OR′, wherein R′ is H, a hydroxy-protecting group, or a solid support, the hydroxy-protecting group is preferably —C(O)CH₂CH₂C(O)OH or 4,4′-dimethoxytrityl;
m1=0, 1, 2, 3, 4, 5, 6, 7, 8, 9 or 10;
n1=0, 1, 2, 3, 4, 5, 6, 7, 8, 9 or 10.

In another aspect, the present invention relates to a conjugated group comprising N-acetylgalactosamine, wherein the conjugated group is shown as formula (I′):

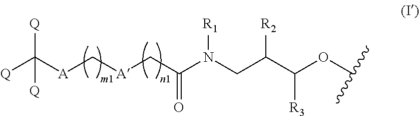

wherein
[symbol] represents the position of attachment to biomolecules;
Q is independently H,

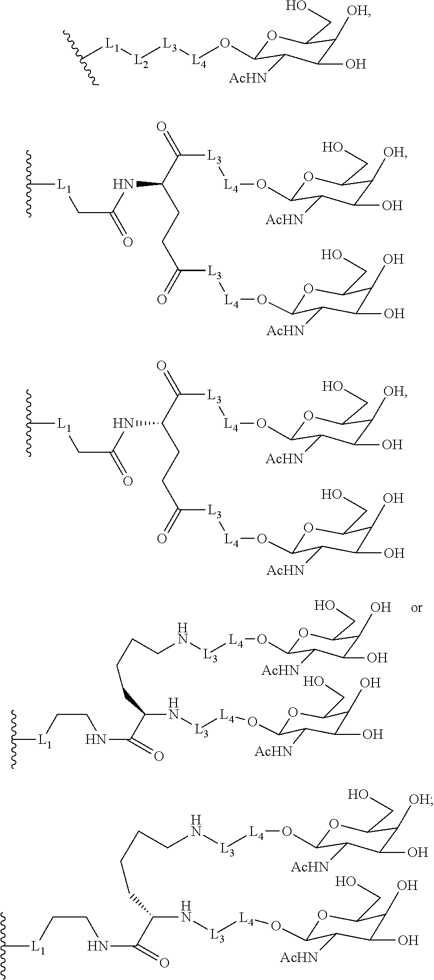

wherein L₁ is bond, —CH₂—, —CH₂CH₂—, —C(O)—, —CH₂O—, —CH₂O—CH₂CH₂O— or —NHC(O)—(CH₂NHC(O))ₐ—;
L₂ is bond or —CH₂CH₂C(O)—;
L₃ is bond, —(NHCH₂CH₂)_{b}—, —(NHCH₂CH₂CH₂)_{b}— or —C(O)CH₂—;
L₄ is —(OCH₂CH₂)ₑ—, —(OCH₂CH₂CH₂)ₑ—, —(OCH₂CH₂CH₂CH₂)—, —(OCH₂CH₂CH₂CH₂CH₂)ₑ— or —NHC(O)—(CH₂)ₐ—;
   wherein a=0, 1, 2 or 3;
   b=1, 2, 3, 4 or 5;
   c=1, 2, 3, 4 or 5;
   d=1, 2, 3, 4, 5, 6, 7 or 8;
A is —CH₂O— or —NHC(O)—;
A′ is bond, —C(O)NH— or —NHC(O)—;
R₁ and R₂ together form —CH₂CH₂O— or —CH₂CH(R)—O—; and R₃ is H;
or R₁ and R₃ together form —C₁₋₂ alkylene-; and R₂ is H;
   wherein R is —OR′, —CH₂OR′ or —CH₂CH₂OR′, wherein R′ is H, a hydroxy-protecting group, or a solid support; the hydroxy-protecting group is preferably —C(O)CH₂CH₂C(O)OH or 4,4′-dimethoxytrityl;
m1=0, 1, 2, 3, 4, 5, 6, 7, 8, 9 or 10;
n1=0, 1, 2, 3, 4, 5, 6, 7, 8, 9 or 10.

In another embodiment, the GalNAc-containing chain comprises a conjugated group of formula (X′), wherein,
Q is independently H,

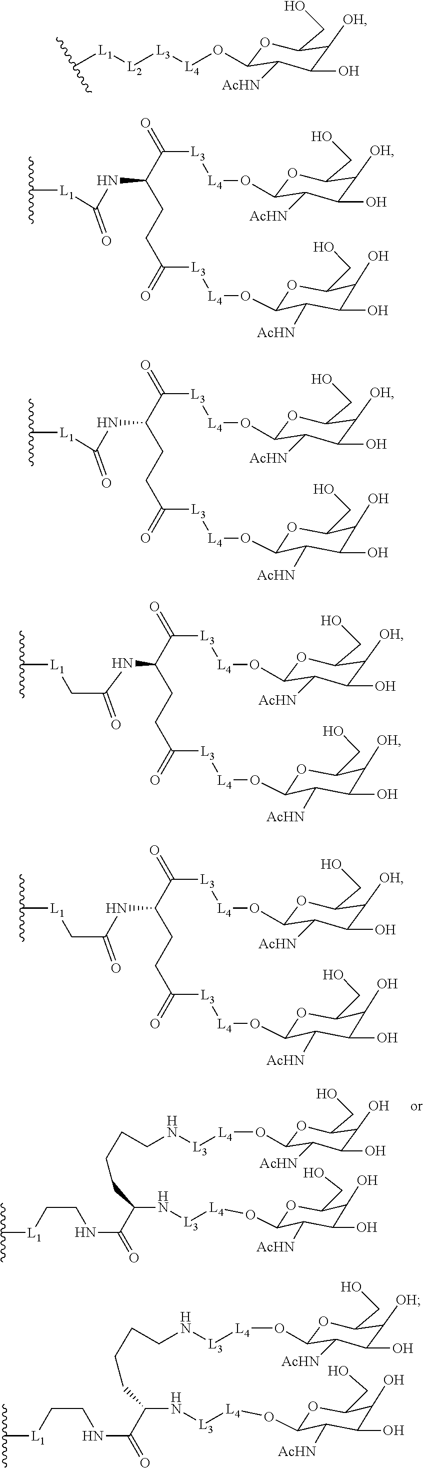

wherein L₁ is bond, —CH₂—, —CH₂CH₂—, —C(O)—, —CH₂O—, —CH₂O—CH₂CH₂O— or —NHC(O)—(CH₂NHC(O))ₐ—;
L₂ is bond or —CH₂CH₂C(O)—;
L₃ is bond, —(NHCH₂CH₂)_{b}—, —(NHCH₂CH₂CH₂)_{b}— or —C(O)CH₂—;
L₄ is —(OCH₂CH₂)ₑ—, —(OCH₂CH₂CH₂)ₑ—, —(OCH₂CH₂CH₂CH₂)ₑ—, —(OCH₂CH₂CH₂CH₂CH₂)ₑ— or —NHC(O)—(CH₂)ₐ—;
   wherein a=0, 1, 2 or 3;
   b=1, 2, 3, 4 or 5;
   c=1, 2, 3, 4 or 5;
   d=1, 2, 3, 4, 5, 6, 7 or 8;
A is bond, —CH₂O— or —NHC(O)—;
A′ is bond, —C(O)NH—, —NHC(O)— or —O(CH₂CH₂O)ₑ—;
   wherein e is 1, 2, 3, 4 or 5;
B is bond, —CH₂—, -M-, —CH₂-M- or —C(O)-M-;
   wherein M is

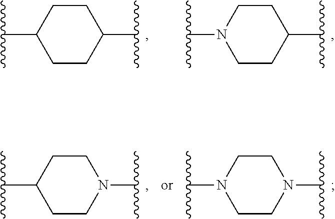 [SMILES: *C1CCC(*)CC1 ; *C1CCN(*)CC1 ; *C1CCN(*)CC1 ; *N1CCN(*)CC1]

R₁ and R₂ together form —CH₂CH₂O— or —CH₂CH(R)—O—; and R₃ is H;
or R₁ and R₃ together form —C₁₋₂ alkylene-; and R₂ is H;
wherein R is —OR′, —CH₂OR′ or —CH₂CH₂OR′, wherein R′ is H, a hydroxy-protecting group, or a solid support; the hydroxy-protecting group is preferably —C(O)CH₂CH₂C(O)OH or 4,4′-dimethoxytrityl;
m1=0, 1, 2, 3, 4, 5, 6, 7, 8, 9 or 10;
n1=0, 1, 2, 3, 4, 5, 6, 7, 8, 9 or 10.

In another embodiment, the GalNAc-containing chain comprises a conjugated group of formula (X′), wherein:
Q is independently H,

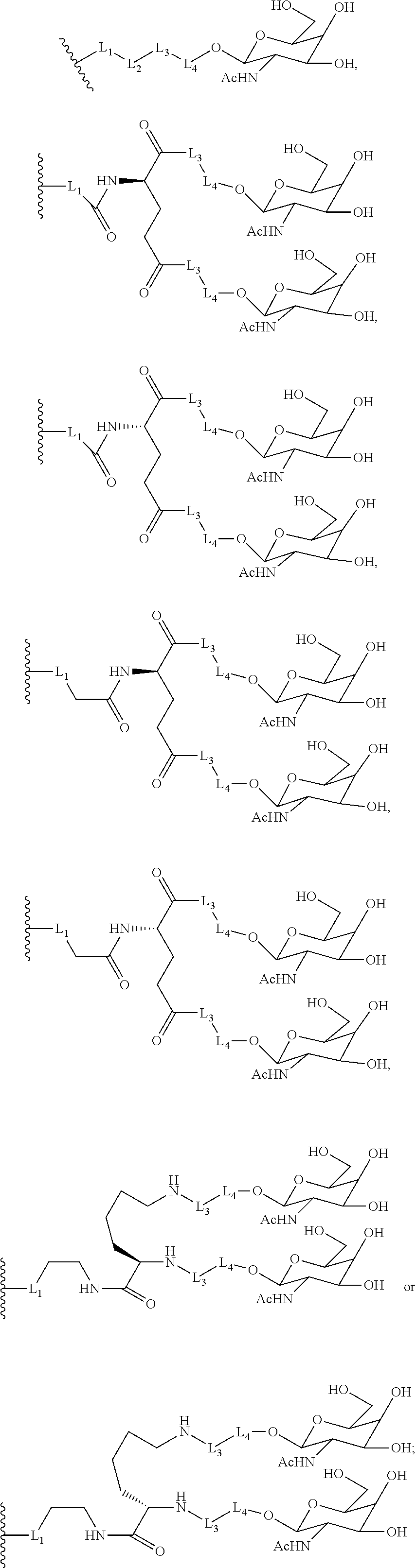

wherein L₁ is bond, —CH₂—, —CH₂CH₂—, —C(O)—, —CH₂O—, —CH₂O—CH₂CH₂O— or —NHC(O)—(CH₂NHC(O))ₐ—;
L₂ is bond or —CH₂CH₂C(O)—;
L₃ is bond, —(NHCH₂CH₂)_{b}—, —(NHCH₂CH₂CH₂)_{b}— or —C(O)CH₂—;
L₄ is —(OCH₂CH₂)ₑ—, —(OCH₂CH₂CH₂)ₑ—, —(OCH₂CH₂CH₂CH₂)ₑ—, —(OCH₂CH₂CH₂CH₂CH₂)ₑ— or —NHC(O)—(CH₂)ₐ—;
   wherein a=0, 1, 2 or 3;
   b=1, 2, 3, 4 or 5;
   c=1, 2, 3, 4 or 5;
   d=1, 2, 3, 4, 5, 6, 7 or 8;
A is bond, —CH₂O— or —NHC(O)—;
A′ is —O(CH₂CH₂O)ₑ—;
   wherein e is 1, 2, 3, 4 or 5;
B is bond, —CH₂—, -Q(O)—, -M-, —CH₂-M- or —C(O)-M-;
   wherein M is

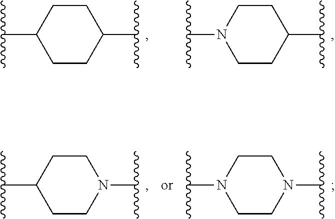 [SMILES: *C1CCC(*)CC1 ; *C1CCN(*)CC1 ; *C1CCN(*)CC1 ; *N1CCN(*)CC1]

R₁ and R₂ together form —CH₂CH₂O— or —CH₂CH(R)—O—; and R₃ is H;
or R₁ and R₃ together form —C₁₋₂ alkylene-; and R₂ is H;
wherein R is —OR′, —CH₂OR′ or —CH₂CH₂OR′, wherein R′ is H, a hydroxy-protecting group, or a solid support, the hydroxy-protecting group is preferably —C(O)CH₂CH₂C(O)OH or 4,4′-dimethoxytrityl;
m1=0, 1, 2, 3, 4, 5, 6, 7, 8, 9 or 10;
n1=0, 1, 2, 3, 4, 5, 6, 7, 8, 9 or 10.

Any technical solution or any combination thereof in any of the above specific embodiments can be combined with any technical solution or any combination thereof in other specific embodiments. For example, any one of the technical solutions for X₁ or any combination thereof can be combined with any one of the technical solutions for X₂, X₃, T, X and L, and Z or any combination thereof. The present invention is intended to include all combinations of these technical solutions, which are not listed one by one due to space limitations.

The present invention also provides a vector comprising a nucleotide sequence encoding the siRNA of the invention. The vector of the present invention can amplify or express a nucleotide encoding the siRNA of the invention linked thereto.

For example, an siRNA targeting PCSK9 can be expressed from a transcription unit inserted into a DNA or RNA vector. Expression can be transient (within hours to weeks) or sustained (weeks to months or longer), depending on the particular construct used and the target tissue or cell type. A nucleotide encoding a siRNA can be introduced into a linear construct, a circular plasmid, or a viral vector. A nucleotide encoding a siRNA can be stably expressed by integration into the cell genome, or can be stably inherited and expressed extrachromosomally. Generally speaking, a vector expressing a siRNA is usually a DNA plasmid or a viral vector.

Viral vector systems comprising a sequence encoding a siRNA include, but are not limited to: (a) adenoviral vectors; (b) retroviral vectors; (c) adeno-associated viral vectors; (d) herpes simplex virus vectors; (e) SV40 vectors; (f) polyomavirus vectors; (g) papillomavirus vectors; (h) picornavirus vectors; (i) poxvirus vectors; and (j) helper virus-dependent or gutless adenoviral vectors.

The present invention also provides a cell comprising a siRNA or vector of the invention, wherein the siRNA or vector of the invention can be transcribed in the cell.

The present invention specifically relates to the following technical solutions:

A1. A compound of formula (I), or a pharmaceutically acceptable salt, tautomer or stereoisomer thereof:

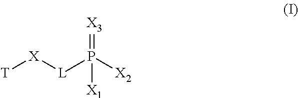

wherein,
X₁ is selected from ORₐ, OP₁, SP₁ or NR_{b}R_{c};
Rₐ is selected from H, C₁₋₆ alkyl, C₁₋₆ haloalkyl, C₂₋₆ alkenyl or C₂₋₆ alkynyl, which is optionally deuterated or fully deuterated;
R_{b} and R_{c} is independently selected from H, C₁₋₆ alkyl or C₁₋₆ haloalkyl; R_{b} and R_{c} may optionally be substituted with D, C₆₋₁₀ aryl or 5-10-membered heteroaryl, or fully deuterated;
X₂ is OH, OP₁ or SP₁, or X₂ is a nucleoside moiety, which connects to P via a hydroxyl or a sulfydryl on the 2′, 3′ or 5′ carbon of the ribose;
X₃ is independently selected from O or S;
T is selected from

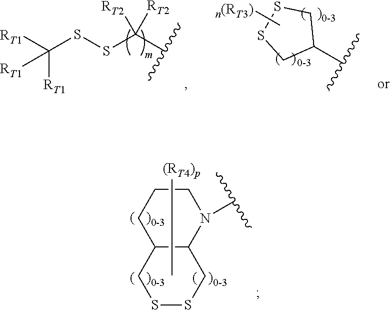

each R_{T1} is independently selected from H, D, halogen, CN, C₁₋₆ alkyl, C₁₋₆ haloalkyl, C₁₋₆ haloalkyl, C₂₋₆ alkenyl, C₂₋₆ alkynyl or GalNAc-containing chain, which is optionally deuterated or fully deuterated;
each R_{T2} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
each R_{T3} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
each R_{T4} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
m is 0, 1, 2, 3, 4 or 5;
n is 0, 1, 2, 3, 4 or 5;
p is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₆ alkyl or C₁₋₆ haloalkyl, which is optionally deuterated or fully deuterated;
L is —Ar—(CH₂)₁₋₆—O—, wherein each CH₂ may optionally be substituted with R #; R # is selected from H, D, halogen, CN, C₁₋₆ alkyl, C₁₋₆ haloalkyl, C₂₋₆ alkenyl or C₂₋₆ alkynyl, which is optionally deuterated or fully deuterated;
Ar in L is linked to X; the oxygen atom in L is linked to the phosphorus atom;
Ar is selected from C₃₋₁₀ cycloalkyl, 3-10-membered heterocyclyl, C₆₋₁₀ aryl or 5-14-membered heteroaryl; C₃₋₁₀ cycloalkyl, 3-10-membered heterocyclyl, C₆₋₁₀ aryl or 5-10-membered heteroaryl may optionally be substituted with 1, 2, 3, 4 or 5 R*;
R* is selected from H, D, halogen, CN, C₁₋₆ alkyl, C₁₋₆ haloalkyl, C₂₋₆ alkenyl or C₂₋₆ alkynyl, which is optionally deuterated or fully deuterated;
wherein P₁ is selected from a protecting group.

A2. The compound of formula (I) of technical solution A1, or a pharmaceutically acceptable salt, tautomer or stereoisomer thereof, wherein,
X₁ is selected from OR, OP₁, SP₁ or NR_{b}R_{c};
Rₐ is selected from H, C₁₋₄ alkyl or C₁₋₄ haloalkyl, which is optionally deuterated or fully deuterated;
R_{b} and R_{c} are independently selected from H, C₁₋₄ alkyl or C₁₋₄ haloalkyl; and R_{b} and R_{c} may optionally be substituted with D, C₆₋₁₀ aryl, which is optionally deuterated or fully deuterated;
X₂ is OH, OP₁ or SP₁, or X₂ is a nucleoside moiety, which connects to P via a hydroxyl or a sulfydryl on the 2′, 3′ or 5′ carbon of the ribose;
X₃ is independently selected from O or S;
T is selected from

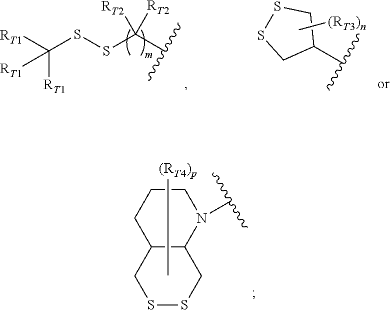

each R_{T1} is independently selected from H, D, C₁₋₄ alkyl, C₁₋₄ haloalkyl or GalNAc-containing chain, which is optionally deuterated or fully deuterated;
each R_{T2} is independently selected from H, D, C₁₋₄ alkyl or C₁₋₄ haloalkyl, which is optionally deuterated or fully deuterated;
each R_{T3} is independently selected from H, D, C₁₋₄ alkyl or C₁₋₄ haloalkyl, which is optionally deuterated or fully deuterated;
each R_{T4} is independently selected from H, D, C₁₋₄ alkyl or C₁₋₄ haloalkyl, which is optionally deuterated or fully deuterated;
m is 0, 1, 2 or 3;
n is 0, 1, 2 or 3;
p is 0, 1, 2 or 3;
X is selected from bond, —O—, —S—, —OC(O)NR_{X1}—, —NR_{X1}C(O)O—, —C(O)O—, —OC(O)—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is —Ar—(CH₂)₁₋₄—O—, wherein each CH₂ may optionally be substituted with 1 or 2 R #; R # is selected from H, D, C₁₋₄ alkyl or C₁₋₄ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₄ alkyl or C₁₋₄ haloalkyl, which is optionally deuterated or fully deuterated;
wherein P₁ is selected from a protecting group.

A3. The compound of formula (I) of technical solutions A1 or A2, or a pharmaceutically acceptable salt, tautomer or stereoisomer thereof, wherein,
X₁ is selected from ORₐ, OP₁, SP₁ or NR_{b}R_{c};
Rₐ is selected from H, C₁₋₄ alkyl or C₁₋₄ haloalkyl, which is optionally deuterated or fully deuterated;
R_{b} and R^{c} are independently selected from H, C₁₋₄ alkyl, C₁₋₄ haloalkyl; R_{b} and R_{c} are optionally substituted with D, phenyl, or fully deuterated;
X₂ is OH, OP₁ or SP₁, or X₂ is a nucleoside moiety, which connects to P via a hydroxyl or a sulfydryl on the 2′, 3′ or 5′ carbon of the ribose;
X₃ is independently selected from O or S;
T is selected from

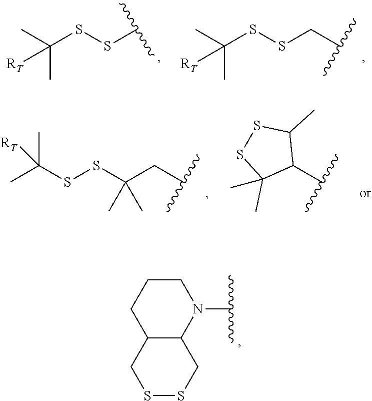

wherein R_{T} is selected from H, D, CH₃ or GalNAc-containing chain, which is optionally deuterated or fully deuterated;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is selected from

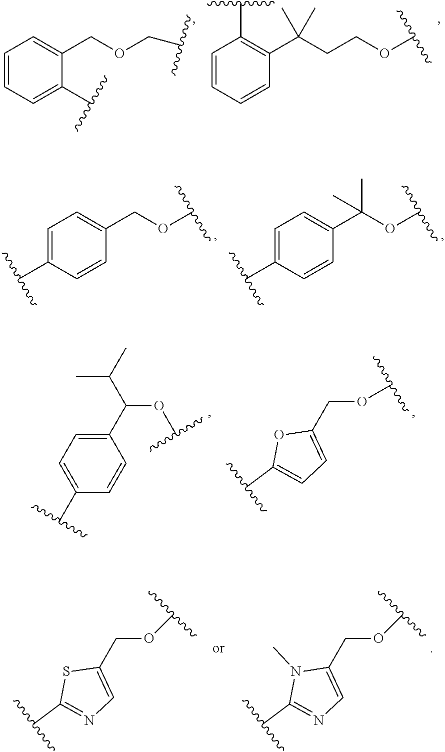

wherein P₁ is selected from a protecting group.

A4. The compound of formula (I) of any one of technical solutions A1-A3, or a pharmaceutically acceptable salt, tautomer or stereoisomer thereof, wherein,
X₁ is selected from OH, OP₁, SP₁ or

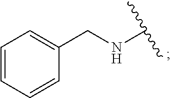 [SMILES: *NCc1ccccc1]

X₂ is OH, OP₁ or SP₁, or X₂ is a nucleoside moiety, which connects to P via a hydroxyl or a sulfydryl on the 2′, 3′ or 5′ carbon of the ribose;
X₃ is independently selected from O or S;
T is selected from

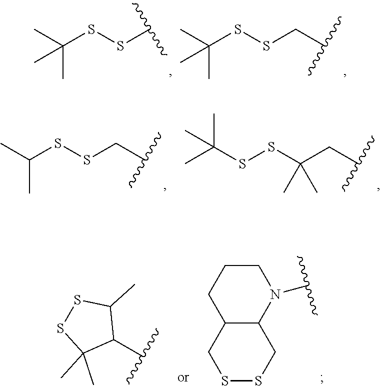

X is selected from bond, —O—, —NHC(O)O—, —OC(O)NH—, —N(CH₃)C(O)O— or —C(O)O—;
L is selected from

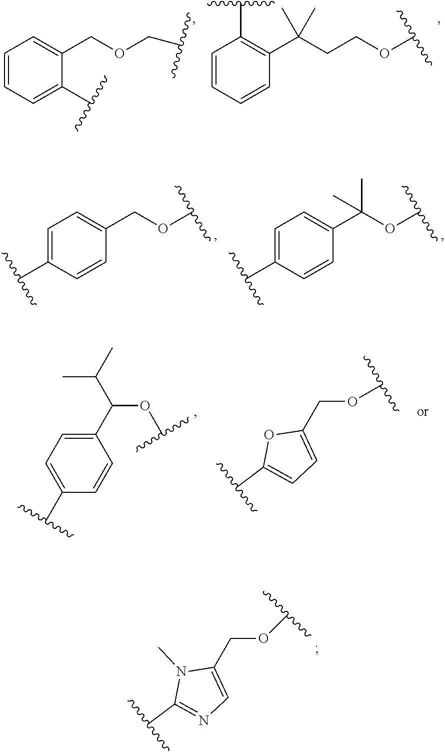

wherein P₁ is selected from a protecting group.

A5. The compound of formula (I) of any one of technical solutions A1-A4, or a pharmaceutically acceptable salt, tautomer or stereoisomer thereof, wherein the compound of formula (I) has a structure as follows:

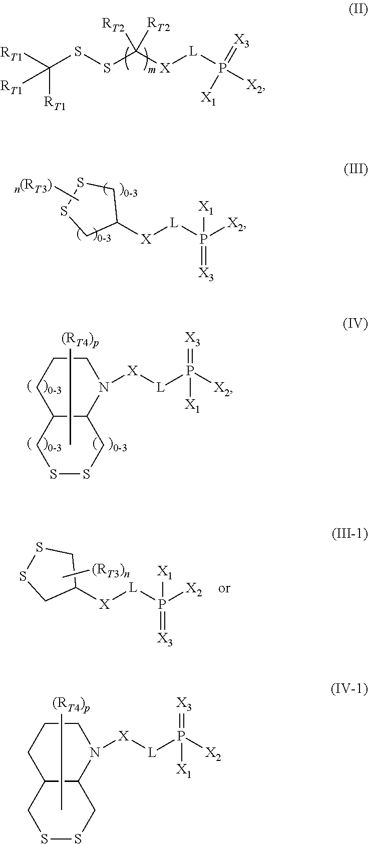

wherein, each group is defined in technical solutions A1-A4.

A6. The compound of formula (I) of technical solution A, or a pharmaceutically acceptable salt, tautomer or stereoisomer thereof, wherein the compound of formula (I) has a structure of formula (II):

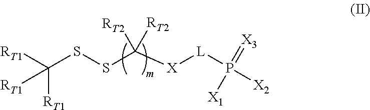

wherein,
X₁ is selected from ORₐ or NR_{b}R_{c};
Rₐ is selected from H, C₁₋₆ alkyl or C₁₋₆ haloalkyl, which is optionally deuterated or fully deuterated;
R_{b} and R_{c} is selected from H, C₁₋₆ alkyl or C₁₋₆ haloalkyl; R_{b} and R_{c} may optionally be substituted with D, C₆₋₁₀ aryl or 5-10-membered heteroaryl, or fully deuterated;
X₂ is OH, OP₁ or SP₁, or X₂ is a nucleoside moiety, which connects to P via a hydroxyl or a sulfydryl on the 2′, 3′ or 5′ carbon of the ribose;
X₃ is independently selected from O or S;
each R_{T1} is independently selected from H, D, C₁₋₆ alkyl, C₁₋₆ haloalkyl, C₂₋₆ alkenyl, C₂₋₆ alkynyl or GalNAc-containing chain, which is optionally deuterated or fully deuterated;
each R_{T2} is independently selected from H, D, C₁₋₆ alkyl, C₁₋₆ haloalkyl, C₂₋₆ alkenyl or C₂₋₆ alkynyl, which is optionally deuterated or fully deuterated;
m is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₆ alkyl or C₁₋₆ haloalkyl, which is optionally deuterated or fully deuterated;
L is —Ar—(CH₂)₁₋₆—O—, wherein each CH₂ may optionally be substituted with 1 or 2 R #; R # is selected from H, D, C₁₋₆ alkyl or C₁₋₆ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2, 3, 4 or 5 R*;
R* is selected from C₁₋₆ alkyl, C₁₋₆ haloalkyl, C₂₋₆ alkenyl or C₂₋₆ alkynyl, which is optionally deuterated or fully deuterated;
wherein P₁ is selected from a protecting group.

A7. The compound of formula (I) of technical solution A6, or a pharmaceutically acceptable salt, tautomer or stereoisomer thereof, wherein,
X₁ is selected from ORₐ or NR_{b}R_{c};
Rₐ is selected from H, C₁₋₄ alkyl or C₁₋₄ haloalkyl, which is optionally deuterated or fully deuterated;
R_{b} and R_{c} is independently selected from H, C₁₋₄ alkyl, C₁₋₄ haloalkyl; R_{b} and R_{c} may optionally be substituted with D, C₆₋₁₀ aryl, or fully deuterated;
X₂ is OH, OP₁ or SP₁, or X₂ is a nucleoside moiety, which connects to P via a hydroxyl or a sulfydryl on the 2′, 3′ or 5′ carbon of the ribose;
X₃ is independently selected from O or S;
each R_{T1} is independently selected from H, D, C₁₋₄ alkyl, C₁₋₄ haloalkyl or GalNAc-containing chain, which is optionally deuterated or fully deuterated;
each R_{T2} is independently selected from H, D, C₁₋₄ alkyl or C₁₋₄ haloalkyl, which is optionally deuterated or fully deuterated;
m is 0, 1, 2 or 3;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is —Ar—(CH₂)₁₋₄—O—, wherein each CH₂ may optionally be substituted with 1 or 2 R #; R # is selected from H, D, C₁₋₄ alkyl or C₁₋₄ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₄ alkyl or C₁₋₄ haloalkyl, which is optionally deuterated or fully deuterated;
wherein P₁ is selected from a protecting group.

A8. The compound of formula (I) of technical solutions A6 or A7, or a pharmaceutically acceptable salt, tautomer or stereoisomer thereof, wherein,
X₁ is selected from ORₐ or NR_{b}R_{c};
Rₐ is selected from H, C₁₋₄ alkyl or C₁₋₄ haloalkyl, which is optionally deuterated or fully deuterated;
R_{b} and R_{c} are independently selected from H, C₁₋₄ alkyl, C₁₋₄ haloalkyl; R_{b} and R_{c} are optionally substituted with D, phenyl, or fully deuterated;
X₂ is OH, OP₁ or SP₁, or X₂ is a nucleoside moiety, which connects to P via a hydroxyl or a sulfydryl on the 2′, 3′ or 5′ carbon of the ribose;
X₃ is independently selected from O or S;

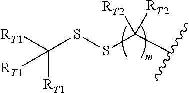

is selected from

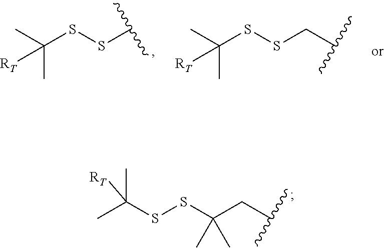

wherein R_{T} is selected from H, D, CH₃ or GalNAc-containing chain, which is optionally deuterated or fully deuterated;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is selected from

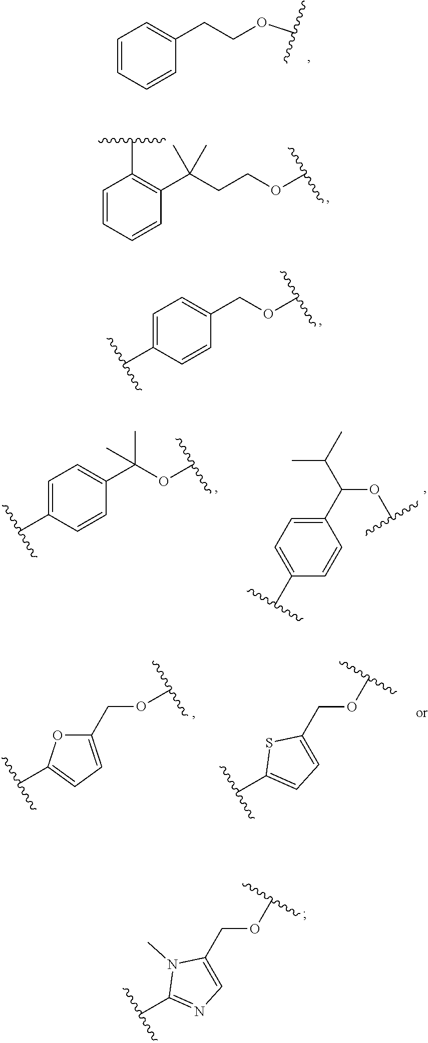

wherein P₁ is selected from a protecting group.

A9. The compound of formula (I) of any one of technical solutions A6-A8, or a pharmaceutically acceptable salt, tautomer or stereoisomer thereof, wherein,

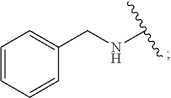 [SMILES: *NCc1ccccc1]

X₁ is selected from OH or
X₂ is OH, OP₁ or SP₁, or X₂ is a nucleoside moiety, which connects to P via a hydroxyl or a sulfydryl on the 2′, 3′ or 5′ carbon of the ribose;
X₃ is independently selected from O or S;

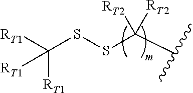

is selected from

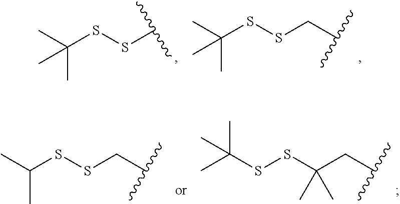

X is selected from bond, —O—, —NHC(O)O—, —OC(O)NH—, —N(CH₃)C(O)O— or —C(O)O—;
L is selected from

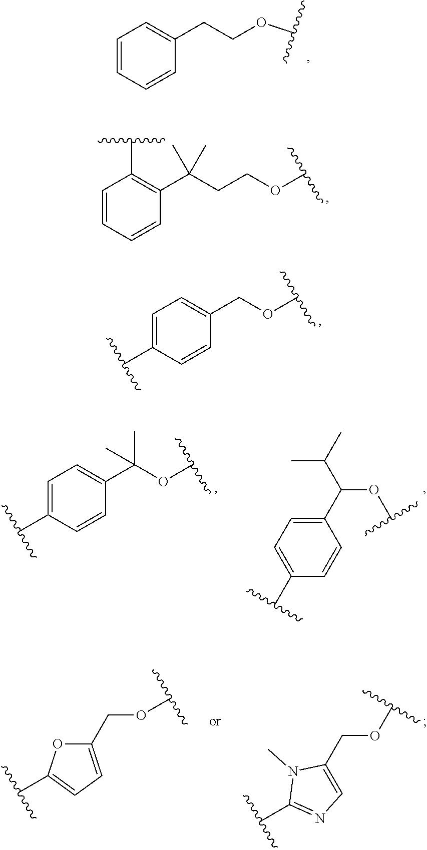

wherein P₁ is selected from a protecting group.

A10. The compound of formula (I) of technical solution A5, or a pharmaceutically acceptable salt, tautomer or stereoisomer thereof, wherein the compound of formula (I) has a structure of formula (II) or IV)

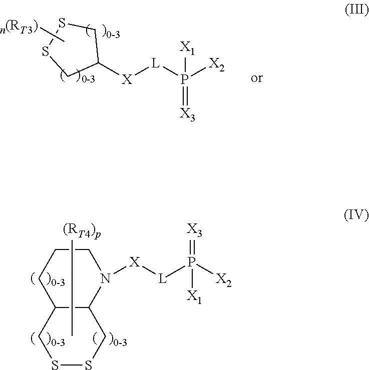

wherein,
X₁ is selected from ORₐ, OP₁, SP₁ or NR_{b}R_{c};
Rₐ is selected from H, C₁₋₆ alkyl or C₁₋₆ haloalkyl, which is optionally deuterated or fully deuterated;
R_{b} and R_{c} is selected from H, C₁₋₆ alkyl or C₁₋₆ haloalkyl; R_{b} and R_{c} may optionally be substituted with D, C₆₋₁₀ aryl or 5-10-membered heteroaryl, or fully deuterated;
X₂ is OH, OP₁ or SP₁, or X₂ is a nucleoside moiety, which connects to P via a hydroxyl or a sulfydryl on the 2′, 3′ or 5′ carbon of the ribose;
X₃ is independently selected from O or S;
each R_{T3} is independently selected from H, D, C₁₋₆ alkyl, C₁₋₆ haloalkyl, C₂₋₆ alkenyl or C₂₋₆ alkynyl, which is optionally deuterated or fully deuterated;
each R_{T4} is independently selected from H, D, C₁₋₆ alkyl, C₁₋₆ haloalkyl, C₂₋₆ alkenyl or C₂₋₆ alkynyl, which is optionally deuterated or fully deuterated;
n is 0, 1, 2, 3, 4 or 5;
p is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₆ alkyl or C₁₋₆ haloalkyl, which is optionally deuterated or fully deuterated;
L is —Ar—(CH₂)₁₋₆—O—, wherein each CH₂ may optionally be substituted with 1 or 2 R #; R # is selected from H, D, C₁₋₆ alkyl or C₁₋₆ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₆ alkyl, C₁₋₆ haloalkyl, C₂₋₆ alkenyl or C₂₋₆ alkynyl, which is optionally deuterated or fully deuterated;
wherein P₁ is selected from a protecting group.

A11. The compound of formula (I) of technical solution A10, or a pharmaceutically acceptable salt, tautomer or stereoisomer thereof, wherein,
X₁ is selected from OR, OP₁, SP₁ or NR_{b}R_{c};
Rₐ is selected from H, C₁₋₄ alkyl or C₁₋₄ haloalkyl, which is optionally deuterated or fully deuterated;
R_{b} and R_{c} is independently selected from H, C₁₋₄ alkyl, C₁₋₄ haloalkyl; R_{b} and R_{c} may be optionally substituted with D or C₆₋₁₀ aryl, or fully deuterated;
X₂ is OH, OP₁ or SP₁, or X₂ is a nucleoside moiety, which connects to P via a hydroxyl or a sulfydryl on the 2′, 3′ or 5′ carbon of the ribose;
X₃ is independently selected from O or S;
each R_{T3} is independently selected from H, D, C₁₋₆ alkyl or C₁₋₆ haloalkyl, which is optionally deuterated or fully deuterated;
each R_{T4} is independently selected from H, D, C₁₋₆ alkyl or C₁₋₆ haloalkyl, which is optionally deuterated or fully deuterated;
n is 0, 1, 2 or 3;
p is 0, 1, 2 or 3;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is —Ar—(CH₂)₁₋₄—O—, wherein each CH₂ may optionally be substituted with 1 or 2 R #; R # is selected from H, D, C₁₋₄ alkyl or C₁₋₄ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₄ alkyl or C₁₋₄ haloalkyl, which is optionally deuterated or fully deuterated;
wherein P₁ is selected from a protecting group.

A12. The compound of formula (I) of technical solutions A10 or A11, or a pharmaceutically acceptable salt, tautomer or stereoisomer thereof, wherein the compound of formula (I) has a structure of formula (III-1) or (IV-1),

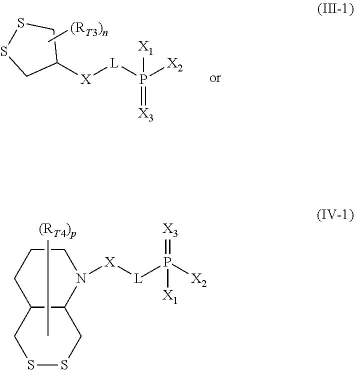

wherein,
X₁ is selected from ORₐ, OP₁, SP₁ or NR_{b}R_{c};
Rₐ is selected from H, C₁₋₄ alkyl or C₁₋₄ haloalkyl, which is optionally deuterated or fully deuterated;
R_{b} and R_{c} are independently selected from H, C₁₋₄ alkyl, C₁₋₄ haloalkyl; R_{b} and R_{c} are optionally substituted with D, phenyl, or fully deuterated;
X₂ is OH, OP₁ or SP₁, or X₂ is a nucleoside moiety, which connects to P via a hydroxyl or a sulfydryl on the 2′, 3′ or 5′ carbon of the ribose;
X₃ is independently selected from O or S;
each R_{T3} is independently selected from H, D, C₁₋₄ alkyl or C₁₋₄ haloalkyl, which is optionally deuterated or fully deuterated;
each R_{T4} is independently selected from H, D, C₁₋₄ alkyl or C₁₋₄ haloalkyl, which is optionally deuterated or fully deuterated;
n is 0, 1, 2 or 3;
p is 0, 1, 2 or 3;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is selected from

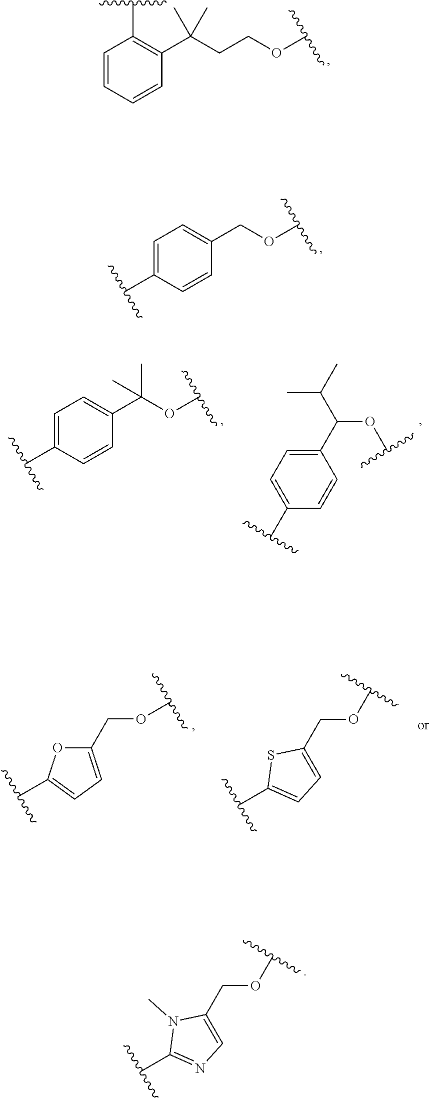

wherein P₁ is selected from a protecting group.

A13. The compound of formula (I) of technical solution A12, or a pharmaceutically acceptable salt, tautomer or stereoisomer thereof wherein
X₁ is selected from OH, OP₁, SP₁ or

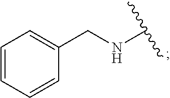 [SMILES: *NCc1ccccc1]

X₂ is OH, OP₁ or SP₁, or X₂ is a nucleoside moiety, which connects to P via a hydroxyl or a sulfydryl on the 2′, 3′ or 5′ carbon of the ribose;
X₃ is independently selected from O or S;
R_{T3} is selected from H, D or CH₃, which is optionally deuterated or fully deuterated;
R_{T4} is H;
n is 3;
p is 0;
X is selected from bond, —O—, —NHC(O)O—, —OC(O)NH—, —N(CH₃)C(O)O— or —C(O)O—;
L is selected from

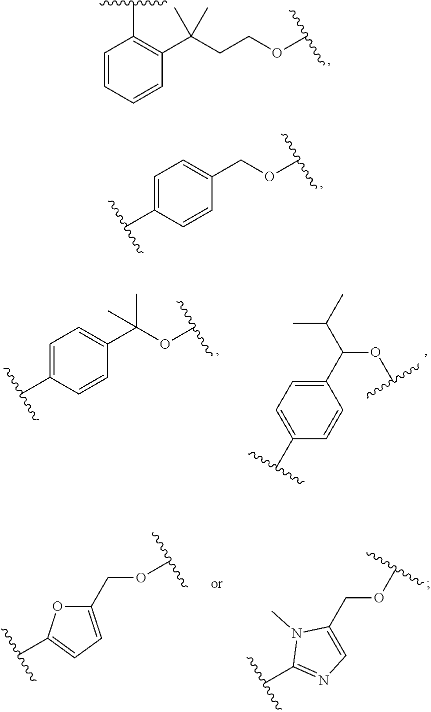

wherein P₁ is selected from a protecting group.

A14. The compound of formula (I) of any one of technical solutions A1-A13, or a pharmaceutically acceptable salt, tautomer or stereoisomer thereof, wherein the compound of formula (I) is selected from:

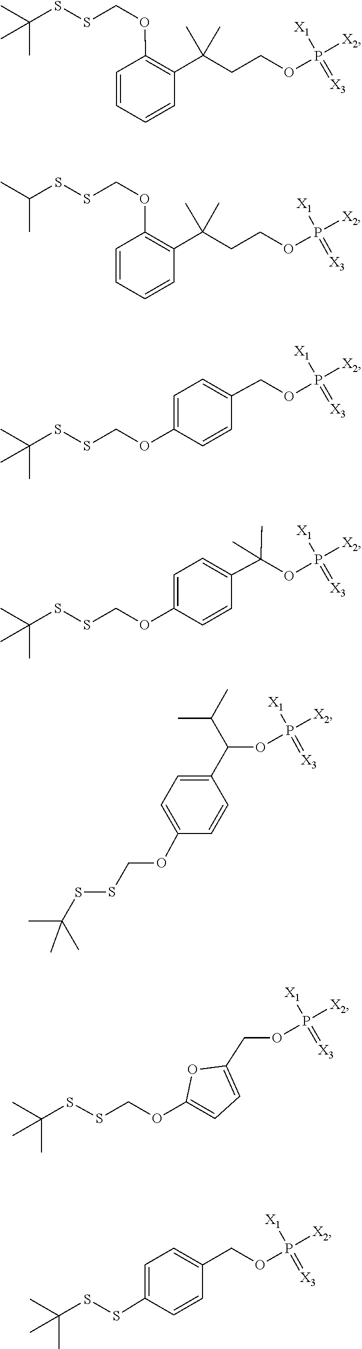

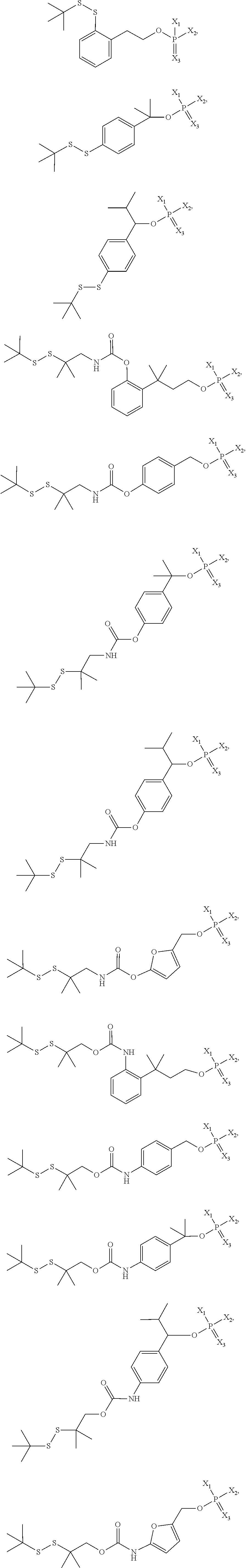

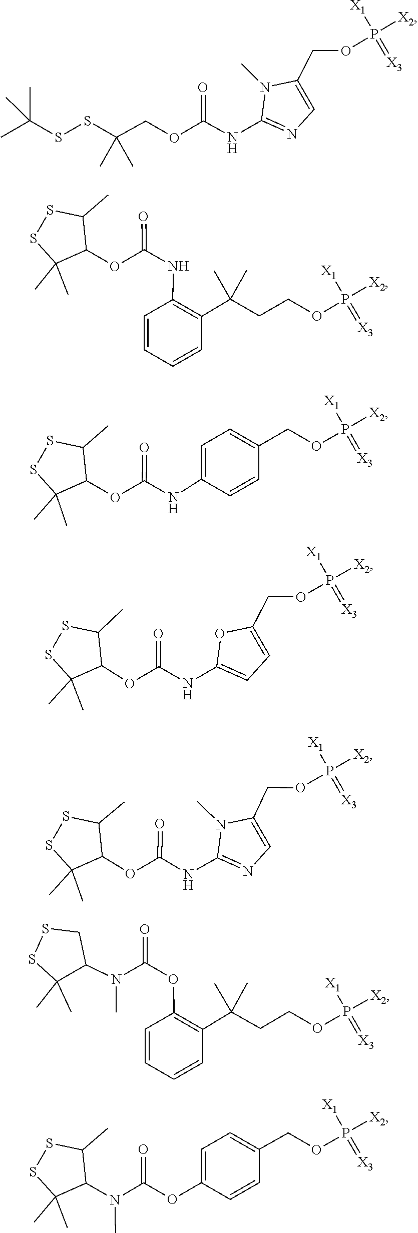

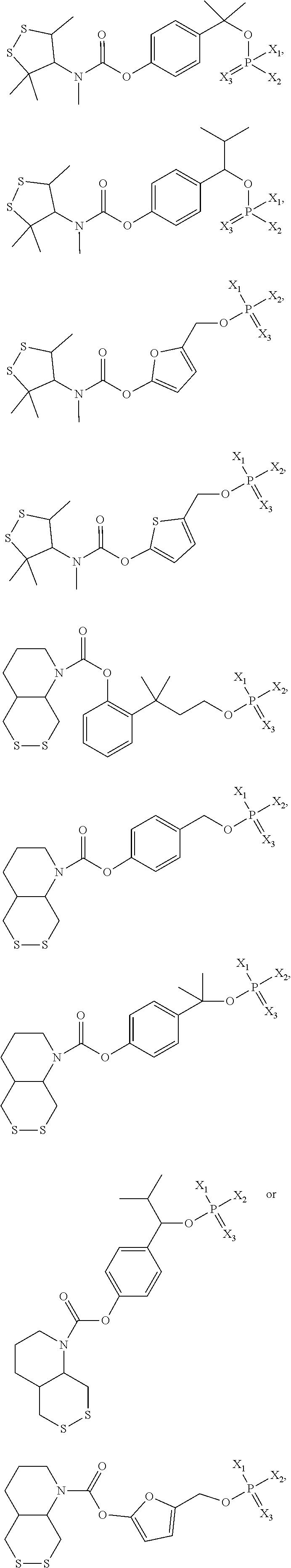

wherein,
X₁ is selected from OH, OP₁ or

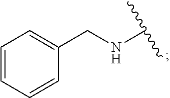 [SMILES: *NCc1ccccc1]

preferably OH or

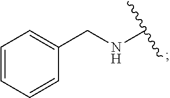 [SMILES: *NCc1ccccc1]

X₂ is OH, OP₁ or SP₁, or a bond connecting to a hydroxyl or a sulfydryl of the 2′ or 3′ carbon of the ribose of the other nucleotide, nucleoside or oligonucleotide;
X₃ is independently selected from O or S;
P₁ is selected from a protecting group;
preferably, the compound of formula I) is selected from:

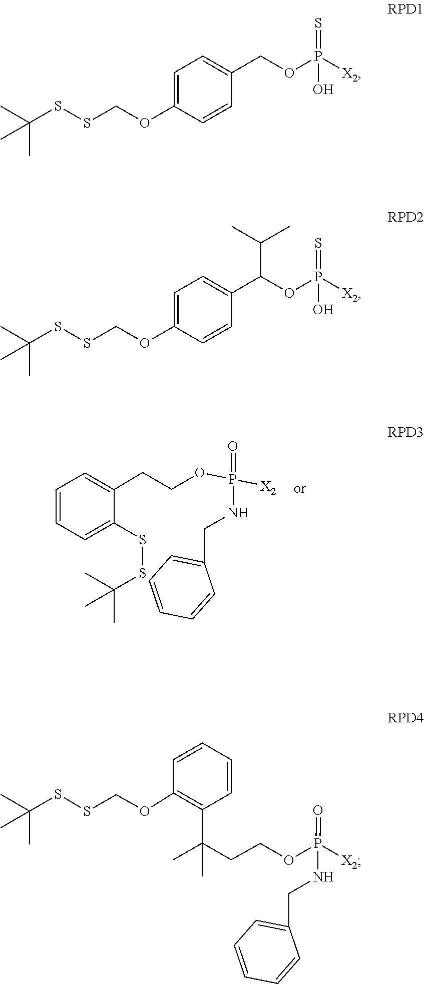

X₂ is a nucleoside moiety, which connects to P via a hydroxyl on the 2′, 3′ or 5′ carbon of the ribose;

A15. The compound of formula (I) of any one of technical solutions A1-A14, or a pharmaceutically acceptable salt, tautomer or stereoisomer thereof, wherein P₁ is selected from a hydroxyl protecting group, e.g., trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), p-methoxybenzyloxymethyl (PMBM), —C(O)CH₂CH₂C(O)OH or 4,4′-dimethoxytrityl, preferably —P(OCH₂CH₂CN)(N(iPr)₂) or —C(O)CH₂CH₂C(O)OH.

A16. An oligonucleotide comprising one, two or three compounds of formula (Ia), or a pharmaceutically acceptable salt, tautomer or stereoisomer thereof:

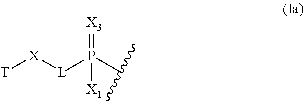

wherein,
[symbol] represents ORₐ or NR_{b}R_{c}, or is a bond connecting to a hydroxyl or a sulfydryl of the 5′ carbon of the ribose of an adjacent nucleotide;
X₁ is selected from ORₐ or NR_{b}R_{c}, or is a bond connecting to a hydroxyl or a sulfydryl of the 2′ or 3′ carbon of the ribose of the other adjacent nucleotide;
and [symbol] and X₁ are not ORₐ or NR_{b}R_{c} simultaneously;
Rₐ is selected from H, C₁₋₆ alkyl, C₁₋₆ haloalkyl, C₂₋₆ alkenyl or C₂₋₆ alkynyl, which is optionally deuterated or fully deuterated;
R_{b} and R_{c} is independently selected from H, C₁₋₆ alkyl or C₁₋₆ haloalkyl; R_{b} and R_{c} may optionally be substituted with D, C₆₋₁₀ aryl or 5-10-membered heteroaryl, or fully deuterated;
X₃ is independently selected from O or S;
T is selected from

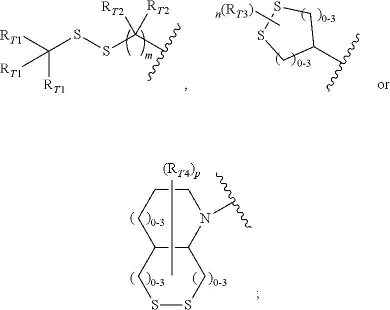

each R_{T1} is independently selected from H, D, halogen, CN, C₁₋₆ alkyl, C₁₋₆ haloalkyl, C₁₋₆ haloalkyl, C₂₋₆ alkenyl, C₂₋₆ alkynyl or GalNAc-containing chain, which is optionally deuterated or fully deuterated;
each R_{T2} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
each R_{T3} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
each R_{T4} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
m is 0, 1, 2, 3, 4 or 5;
n is 0, 1, 2, 3, 4 or 5;
p is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, halogen, C₁₋₆ alkyl or C₁₋₆ haloalkyl, which is optionally deuterated or fully deuterated;
L is —Ar—(CH₂)₁₋₆—O—, wherein each CH₂ may be optionally substituted with R #; R # is selected from H, D, halogen, CN, C₁₋₆ alkyl, C₁₋₆ haloalkyl, C₂₋₆ alkenyl or C₂₋₆ alkynyl, which is optionally deuterated or fully deuterated;
Ar in L is linked to X; the oxygen atom in L is linked to the phosphorus atom;
Ar is selected from C₃₋₁₀ cycloalkyl, 3-10-membered heterocyclyl, C₆₋₁₀ aryl or 5-14-membered heteroaryl; C₃₋₁₀ cycloalkyl, 3-10-membered heterocyclyl, C₆₋₁₀ aryl or 5-10-membered heteroaryl may optionally be substituted with 1, 2, 3, 4 or 5 R*;
R* is selected from H, D, halogen, CN, C₁₋₆ alkyl, C₁₋₆ haloalkyl, C₂₋₆ alkenyl or C₂₋₆ alkynyl, which is optionally deuterated or fully deuterated.

A17. The oligonucleotide of technical solution A16, wherein,
[symbol] represents a bond connecting to a hydroxyl or a sulfydryl of the 5′ carbon of the ribose of an adjacent nucleotide;
X₁ is selected from ORₐ or NR_{b}R_{c}, or is a bond connecting to a hydroxyl or a sulfydryl of the 2′ or 3′ carbon of the ribose of the other adjacent nucleotide;
Rₐ is selected from H, C₁₋₄ alkyl or C₁₋₄ haloalkyl, which is optionally deuterated or fully deuterated;
R_{b} and R_{c} are independently selected from H, C₁₋₄ alkyl or C₁₋₄ haloalkyl; and R_{b} and R_{c} may optionally be substituted with D, C₆₋₁₀ aryl, which is optionally deuterated or fully deuterated;
X₂ and X₃ is independently selected from O or S;
T is selected from

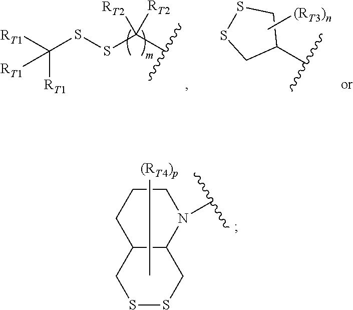

each R_{T1} is independently selected from H, D, C₁₋₄ alkyl, C₁₋₄ haloalkyl or GalNAc-containing chain, which is optionally deuterated or fully deuterated;
each R_{T2} is independently selected from H, D, C₁₋₄ alkyl or C₁₋₄ haloalkyl, which is optionally deuterated or fully deuterated;
each R_{T3} is independently selected from H, D, C₁₋₄ alkyl or C₁₋₄ haloalkyl, which is optionally deuterated or fully deuterated;
each R_{T4} is independently selected from H, D, C₁₋₄ alkyl or C₁₋₄ haloalkyl, which is optionally deuterated or fully deuterated;
m is 0, 1, 2 or 3;
n is 0, 1, 2 or 3;
p is 0, 1, 2 or 3;
X is selected from bond, —O—, —S—, —OC(O)NR_{X1}—, —NR_{X1}C(O)O—, —C(O)O—, —OC(O)—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is —Ar—(CH₂)₁₋₄—O—, wherein each CH₂ may optionally be substituted with 1 or 2 R #; R # is selected from H, D, C₁₋₄ alkyl or C₁₋₄ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₄ alkyl or C₁₋₄ haloalkyl, which is optionally deuterated or fully deuterated.

A18. The olignucleotide of technical solutions A16 or A17, wherein,
[symbol] represents a bond connecting to a hydroxyl or a sulfydryl of the 5′ carbon of the ribose of an adjacent nucleotide;
X₁ is selected from ORₐ or NR_{b}R_{c}, or is a bond connecting to a hydroxyl or a sulfydryl of the 2′ or 3′ carbon of the ribose of the other adjacent nucleotide;
Rₐ is selected from H, C₁₋₄ alkyl or C₁₋₄ haloalkyl, which is optionally deuterated or fully deuterated;
R_{b} and R_{c} are independently selected from H, C₁₋₄ alkyl or C₁₋₄ haloalkyl; R_{b} and R_{c} are optionally substituted with D, phenyl, or fully deuterated;
X₃ is independently selected from O or S;
T is selected from

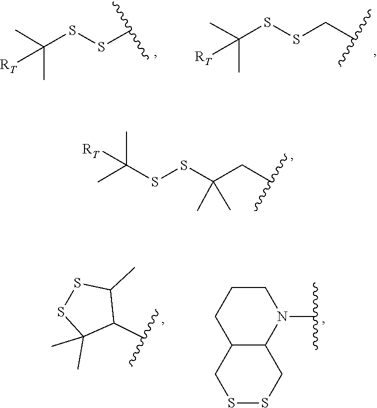

wherein R_{T} is selected from H, D, CH₃ or GalNAc-containing chain, which is optionally deuterated or fully deuterated;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is selected from

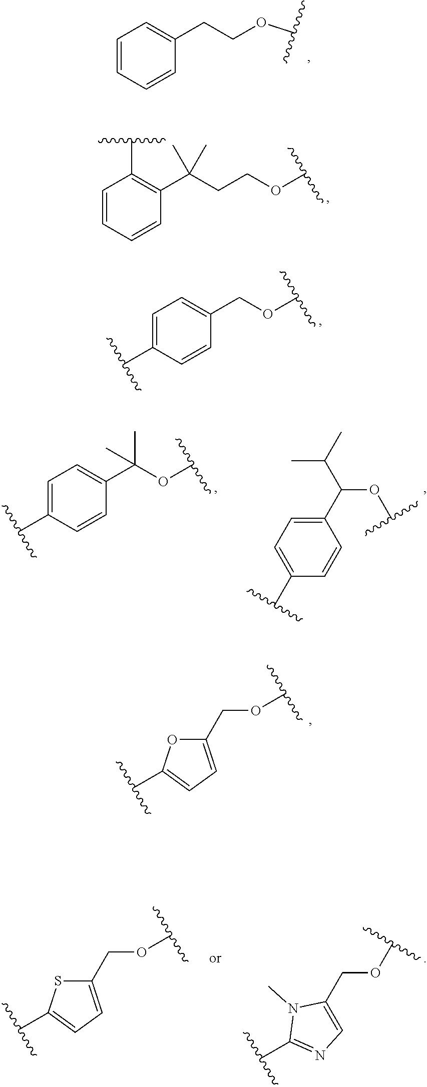

A19. The oligonucleotide of any one of technical solutions A16-A18, wherein,
[symbol] represents a bond connecting to a hydroxyl or a sulfydryl of the 5′ carbon of the ribose of an adjacent nucleotide;
X₁ is selected from OH or

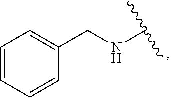 [SMILES: *NCc1ccccc1]

or is a bond connecting to a hydroxyl or a sulfydryl of the 2′ or 3′ carbon of the ribose of the other adjacent nucleotide;
X₃ is selected from O or S;
T is selected from

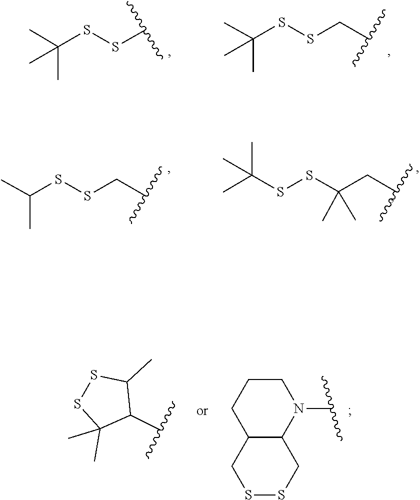

X is selected from bond, —O—, —NHC(O)O—, —OC(O)NH—, —N(CH₃)C(O)O— or —C(O)O—;
L is selected from

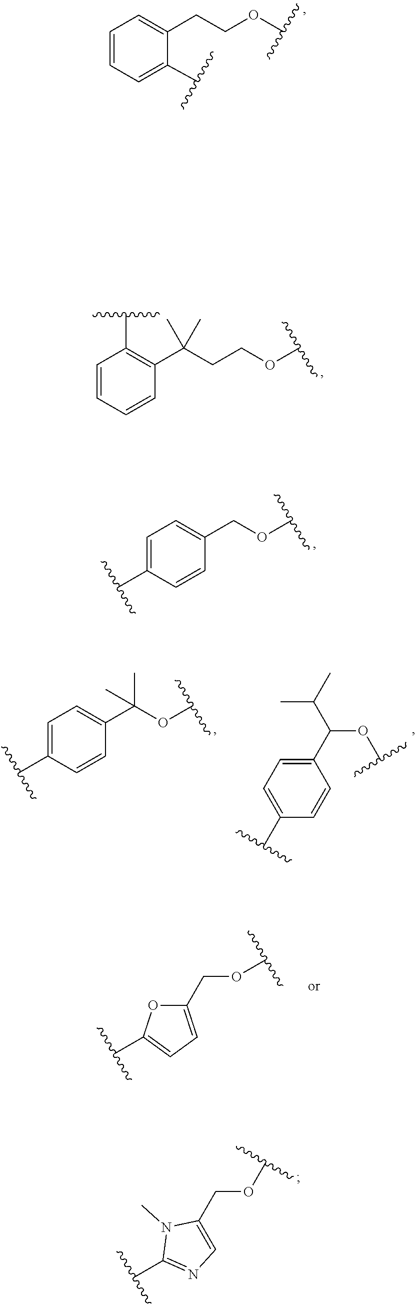

A20. The oligonucleotide of any one of technical solutions A16-A19, wherein the compound of formula (Ia) is selected from the following structures:

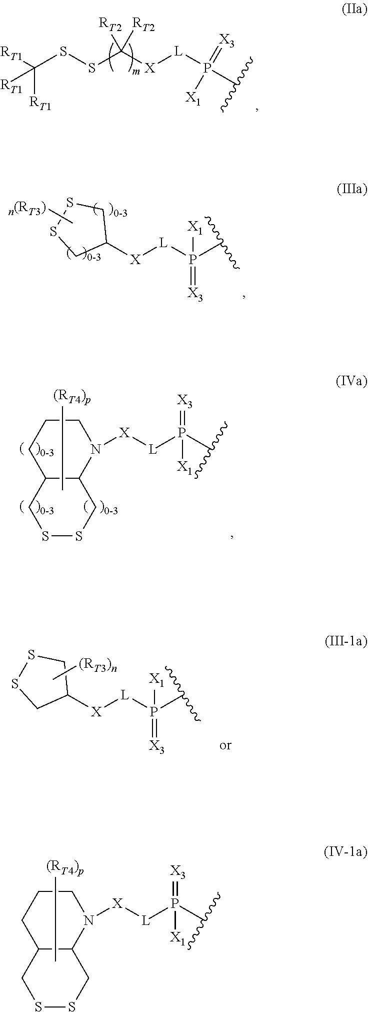

wherein each group is defined as in technical solutions 16-19.

A21. The oligonucleotide of technical solution A20, wherein the compound of formula (Ia) has a structure of formula (IIa):

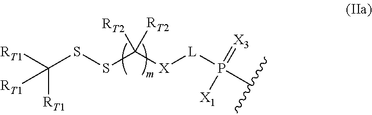

wherein,
[symbol] represents a bond connecting to a hydroxyl or a sulfydryl of the 5′ carbon of the ribose of an adjacent nucleotide;
X₁ is selected from ORₐ or NR_{b}R_{c};
Rₐ is selected from H, C₁₋₆ alkyl or C₁₋₆ haloalkyl, which is optionally deuterated or fully deuterated;
R_{b} and R_{c} are selected from H, C₁₋₆ alkyl or C₁₋₆ haloalkyl; R_{b} and R_{c} may optionally be substituted with D, C₆₋₁₀ aryl or 5-10-membered heteroaryl, or fully deuterated;
X₃ is independently selected from O or S;
each R_{T1} is independently selected from H, D, C₁₋₆ alkyl, C₁₋₆ haloalkyl, C₂₋₆ alkenyl, C₂₋₆ alkynyl or GalNAc-containing chain, which is optionally deuterated or fully deuterated;
each R_{T2} is independently selected from H, D, C₁₋₆ alkyl, C₁₋₆ haloalkyl, C₂₋₆ alkenyl or C₂₋₆ alkynyl, which is optionally deuterated or fully deuterated;
m is 0, 1, 2 or 3;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₆ alkyl or C₁₋₆ haloalkyl, which is optionally deuterated or fully deuterated;
L is —Ar—(CH₂)₁₋₆—O—, wherein each CH₂ may optionally be substituted with 1 or 2 R #; R # is selected from H, D, C₁₋₆ alkyl or C₁₋₆ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₆ alkyl, C₁₋₆ haloalkyl, C₂₋₆ alkenyl or C₂₋₆ alkynyl, which is optionally deuterated or fully deuterated.

A22. The oligonucleotide of technical solution A21, wherein,
[symbol] represents a bond connecting to a hydroxyl or a sulfydryl of the 5′ carbon of the ribose of an adjacent nucleotide;
X₁ is selected from ORₐ or NR_{b}R_{c};
Rₐ is selected from H, C₁₋₄ alkyl or C₁₋₄ haloalkyl, which is optionally deuterated or fully deuterated;
R_{b} and R_{c} is independently selected from H, C₁₋₄ alkyl, C₁₋₄ haloalkyl; R_{b} and R_{c} may optionally be substituted with D, C₆₋₁₀ aryl, or fully deuterated;
X₃ is independently selected from O or S;
each R_{T1} is independently selected from H, D, C₁₋₄ alkyl, C₁₋₄ haloalkyl or GalNAc-containing chain, which is optionally deuterated or fully deuterated;
each R_{T2} is independently selected from H, D, C₁₋₄ alkyl or C₁₋₄ haloalkyl, which is optionally deuterated or fully deuterated;
m is O, 1 or 2;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is —Ar—(CH₂)₁₋₄—O—, wherein each CH₂ may optionally be substituted with 1 or 2 R #; R # is selected from H, D, C₁₋₄ alkyl or C₁₋₄ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₄ alkyl or C₁₋₄ haloalkyl, which is optionally deuterated or fully deuterated.

A23. The oligonucleotide of technical solutions A21 or A22, wherein,
[symbol] represents a bond connecting to a hydroxyl or a sulfydryl of the 5′ carbon of the ribose of an adjacent nucleotide;
X₁ is selected from ORₐ or NR_{b}R_{c};
Rₐ is selected from H, C₁₋₄ alkyl or C₁₋₄ haloalkyl, which is optionally deuterated or fully deuterated;
R_{b} and R_{c} are independently selected from H, C₁₋₄ alkyl, C₁₋₄ haloalkyl; R_{b} and R_{c} are optionally substituted with D or phenyl which is optionally deuterated or fully deuterated;
X₃ is independently selected from O or S;

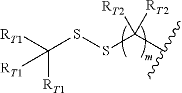

is selected from

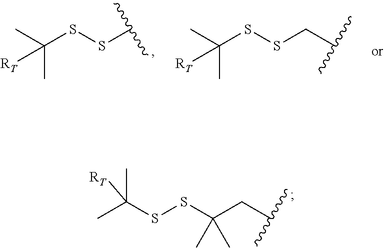

wherein R_{T} is selected from H, D, CH₃ or GalNAc-containing chain, which is optionally deuterated or fully deuterated;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is selected from

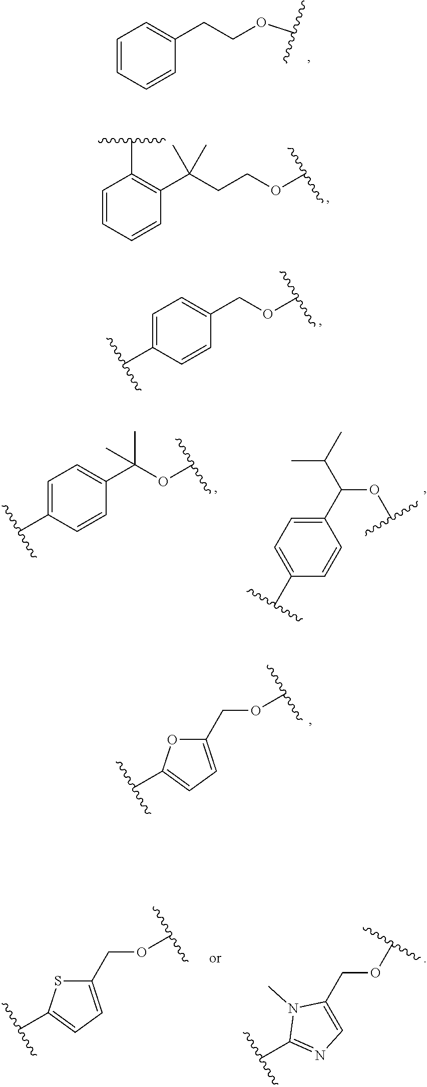

A24. The oligonucleotide of any one of technical solutions A21-A23, wherein,
[symbol] represents a bond connecting to a hydroxyl or a sulfydryl of the 5′ carbon of the ribose of an adjacent nucleotide;
X₁ is selected from OH or

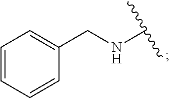 [SMILES: *NCc1ccccc1]

X₃ is selected from O or S;

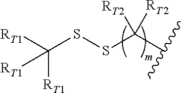

is selected from

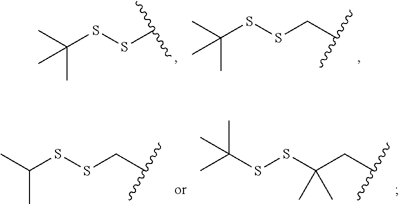

X is selected from bond, —O—, —NHC(O)O—, —OC(O)NH—, —N(CH₃)C(O)O— or —C(O)O—;
L is selected from

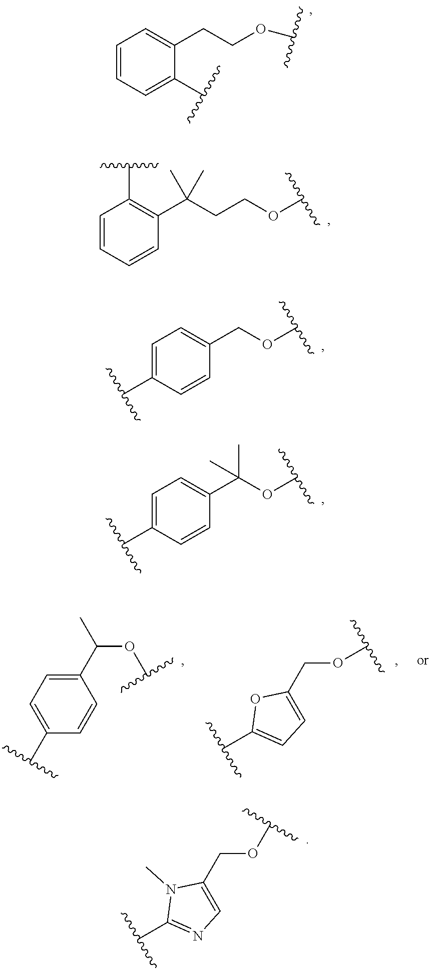

A25. The oligonucleotide of any one of technical solutions A16-A24, wherein, the compound of formula (Ia) is selected from the following structures:

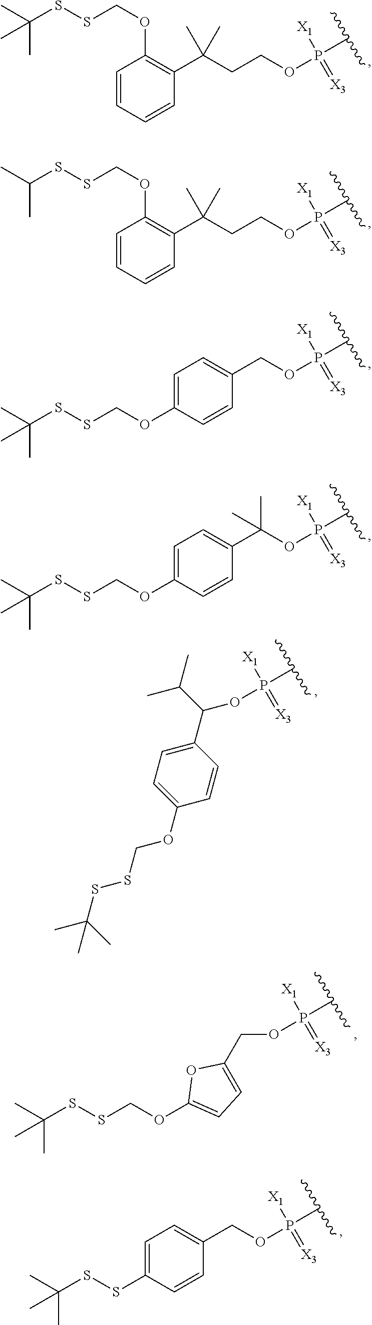

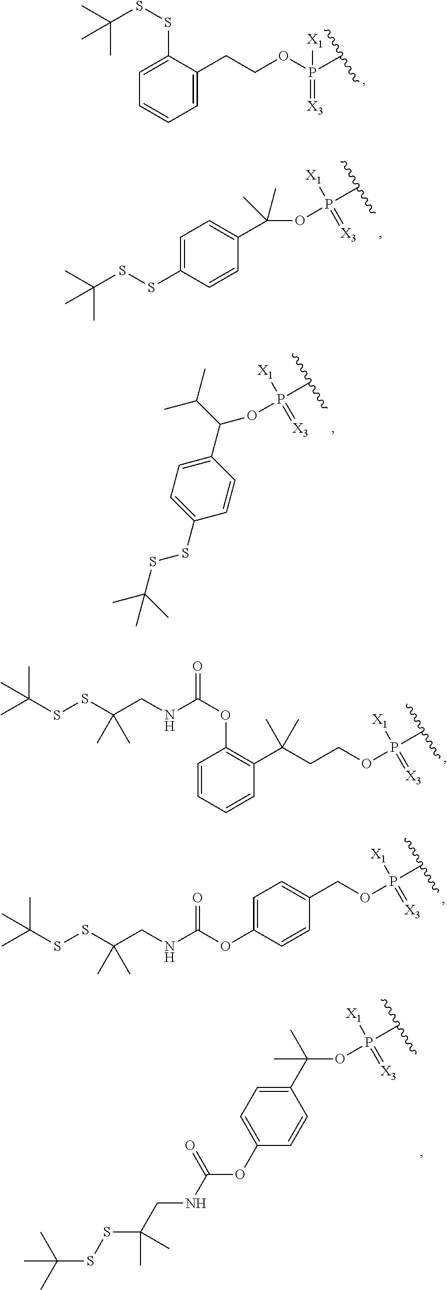

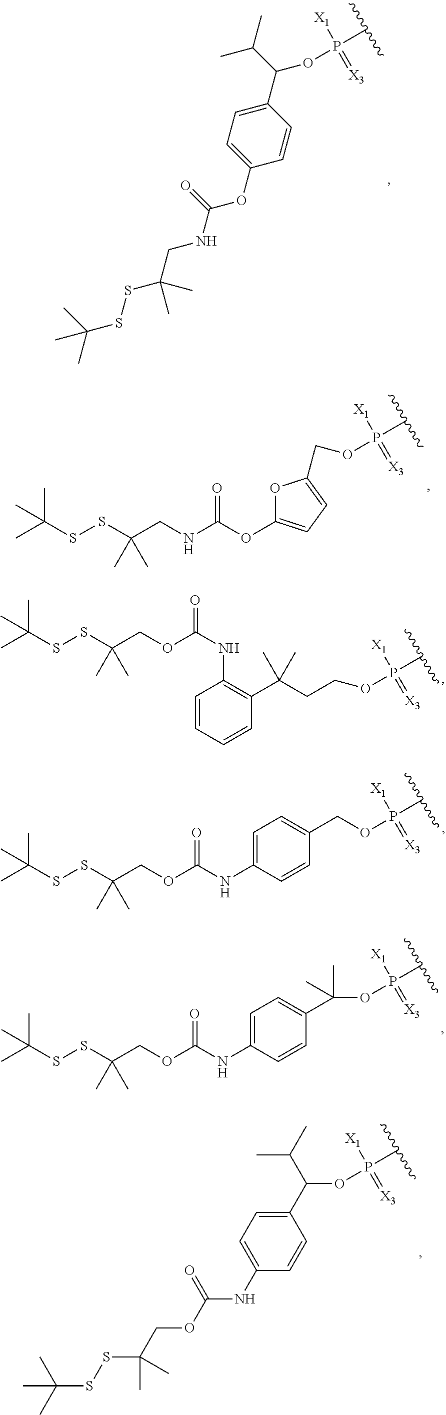

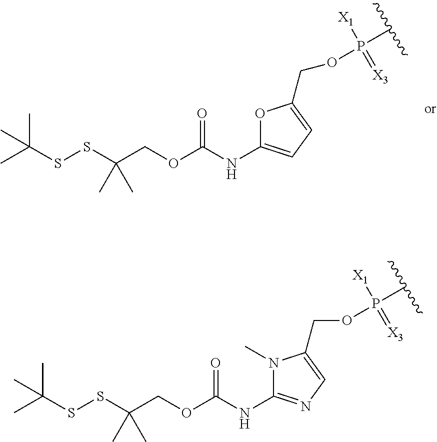

wherein,
[symbol] represents a bond connecting to a hydroxyl or a sulfydryl of the 5′ carbon of the ribose of an adjacent nucleotide;
X₁ is selected from OH or

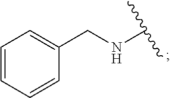 [SMILES: *NCc1ccccc1]

X₃ is selected from O or S;
preferably, the compound of formula (Ia) is selected from:

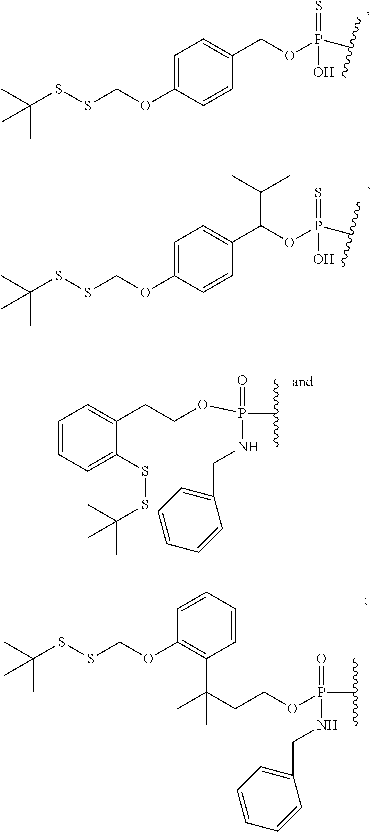

[symbol] represents a bond connecting to a hydroxyl of the 5′ carbon of the ribose of an adjacent nucleotide.

A26. The oligonucleotide of technical solution A20, wherein the compound of formula (Ia) has a structure of formula (IIIa) or (IVa):

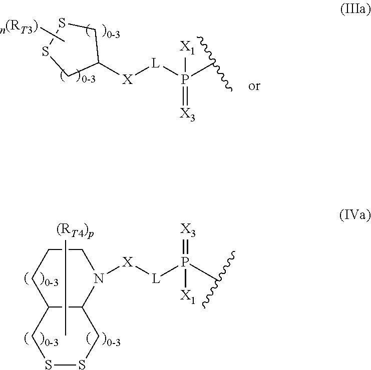

wherein,
[symbol] is selected from ORₐ or NR_{b}R_{c}, or represents a bond connecting to a hydroxyl or a sulfydryl of the 5′ carbon of the ribose of an adjacent nucleotide;
X₁ is selected from ORₐ or NR_{b}R_{c}, or is a bond connecting to a hydroxyl or a sulfydryl of the 2′ or 3′ carbon of the ribose of the other adjacent nucleotide;
and [symbol] and X₁ are not ORₐ or NR_{b}R_{c} simultaneously;
Rₐ is selected from H, C₁₋₆ alkyl or C₁₋₆ haloalkyl, which is optionally deuterated or fully deuterated;
R_{b} and R_{c} is selected from H, C₁₋₆ alkyl or C₁₋₆ haloalkyl; R_{b} and R_{c} may optionally be substituted with D, C₆₋₁₀ aryl or 5-10-membered heteroaryl, or fully deuterated;
X₃ is independently selected from O or S;
each R_{T3} is independently selected from H, D, C₁₋₆ alkyl, C₁₋₆ haloalkyl, C₂₋₆ alkenyl or C₂₋₆ alkynyl, which is optionally deuterated or fully deuterated;
each R_{T4} is independently selected from H, D, C₁₋₆ alkyl, C₁₋₆ haloalkyl, C₂₋₆ alkenyl or C₂₋₆ alkynyl, which is optionally deuterated or fully deuterated;
n is 0, 1, 2, 3, 4 or 5;
p is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₆ alkyl or C₁₋₆ haloalkyl, which is optionally deuterated or fully deuterated;
L is —Ar—(CH₂)₁₋₆—O—, wherein each CH₂ may optionally be substituted with 1 or 2 R #; R # is selected from H, D, C₁₋₆ alkyl or C₁₋₆ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₆ alkyl, C₁₋₆ haloalkyl, C₂₋₆ alkenyl or C₂₋₆ alkynyl, which is optionally deuterated or fully deuterated.

A27. The oligonucleotide of technical solution A26, wherein,
[symbol] is selected from ORₐ or NR_{b}R_{c}, or represents a bond connecting to a hydroxyl or a sulfydryl of the 5′ carbon of the ribose of an adjacent nucleotide;
X₁ is selected from ORₐ or NR_{b}R_{c}, or is a bond connecting to a hydroxyl or a sulfydryl of the 2′ or 3′ carbon of the ribose of the other adjacent nucleotide;
and [symbol] and X₁ are not ORₐ or NR_{b}R_{c} simultaneously;
Rₐ is selected from H, C₁₋₄ alkyl or C₁₋₄ haloalkyl;
R_{b} and R_{c} is independently selected from H, C₁₋₄ alkyl, C₁₋₄ haloalkyl; R_{b} and R_{c} may optionally be substituted with C₆₋₁₀ aryl;
X₂ and X₃ is independently selected from O or S;
each R_{T3} is independently selected from H, C₁₋₆ alkyl or C₁₋₆ haloalkyl;
each R_{T4} is independently selected from H, C₁₋₆ alkyl or C₁₋₆ haloalkyl;
n is 0, 1, 2 or 3;
p is 0, 1, 2 or 3;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl;
L is —Ar—(CH₂)₁₋₄—O—, wherein each CH₂ may optionally be substituted with 1 or 2 R #; R # is selected from H, C₁₋₄ alkyl or C₁₋₄ haloalkyl;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₄ alkyl or C₁₋₄ haloalkyl.

A28. The oligonucleotide of technical solutions A26 or A27, wherein the compound of formula (Ia) has a structure of formula (III-1a) or (IV-1a):

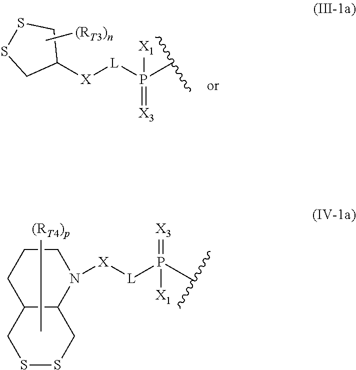

wherein,
[symbol] is selected from ORₐ or NR_{b}R_{c}, or represents a bond connecting to a hydroxyl or a sulfydryl of the 5′ carbon of the ribose of an adjacent nucleotide;
X₁ is selected from ORₐ or NR_{b}R_{c}, or is a bond connecting to a hydroxyl or a sulfydryl of the 2′ or 3′ carbon of the ribose of the other adjacent nucleotide;
and [symbol] and X₁ are not ORₐ or NR_{b}R_{c} simultaneously;
Rₐ is selected from H, C₁₋₄ alkyl or C₁₋₄ haloalkyl, which is optionally deuterated or fully deuterated;
R_{b} and R_{c} are independently selected from H, C₁₋₄ alkyl, C₁₋₄ haloalkyl; R_{b} and R_{c} are optionally substituted with D, phenyl, or fully deuterated;
X₃ is independently selected from O or S;
each R_{T3} is independently selected from H, D, C₁₋₄ alkyl or C₁₋₄ haloalkyl, which is optionally deuterated or fully deuterated;
each R_{T4} is independently selected from H, D, C₁₋₄ alkyl or C₁₋₄ haloalkyl, which is optionally deuterated or fully deuterated;
n is 0, 1, 2 or 3;
p is 0, 1, 2 or 3;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is selected from

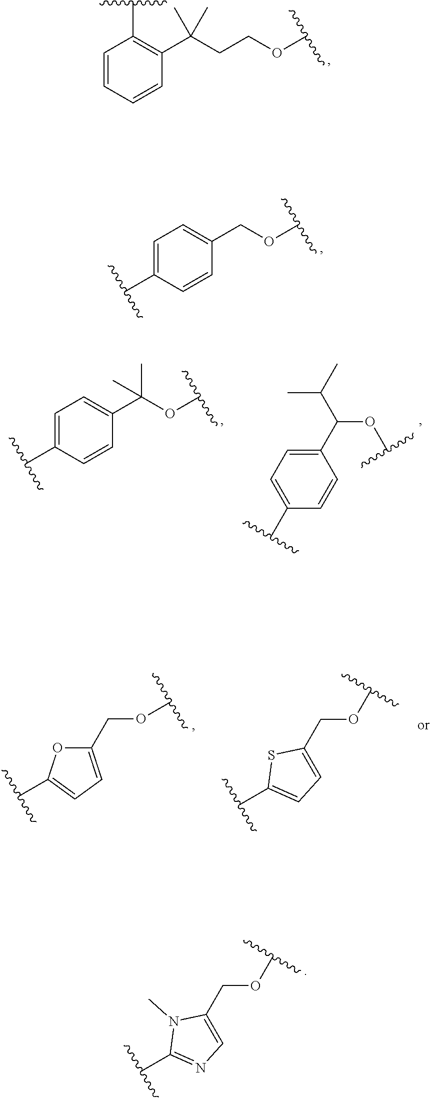

A29. The oligonucleotide of technical solution A28, wherein,
[symbol] is selected from OH or

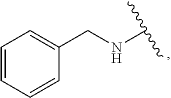 [SMILES: *NCc1ccccc1]

or represents a bond connecting to a hydroxyl or a sulfydryl of the 5′ carbon of the ribose of an adjacent nucleotide;
X₁ is selected from OH or

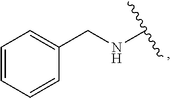 [SMILES: *NCc1ccccc1]

or is a bond connecting to a hydroxyl or a sulfydryl of the 2′ or 3′ carbon of the ribose of the other adjacent nucleotide;
and [symbol] and X₁ are not OH or

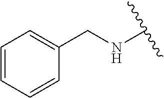 [SMILES: *NCc1ccccc1]

simultaneously;
X₃ is selected from O or S;
R_{T3} is selected from H, D or CH₃, which is optionally deuterated or fully deuterated;
R_{T4} is H;
n is 3;
p is 0;
X is selected from bond, —O—, —NHC(O)O—, —OC(O)NH—, —N(CH₃)C(O)O— or —C(O)O—;
L is selected from

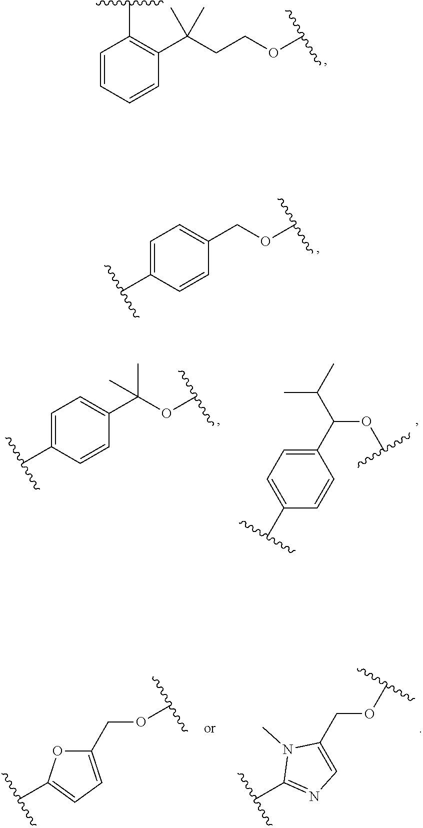

A30. The oligonucleotide of any one of technical solutions A16-A20 and A26-A29, wherein the compound of formula (Ia) is selected from the following structures:

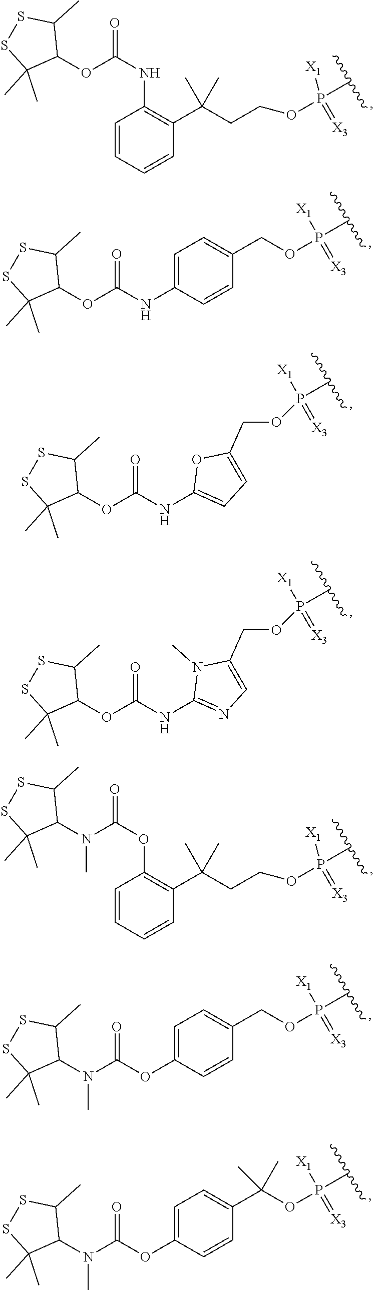

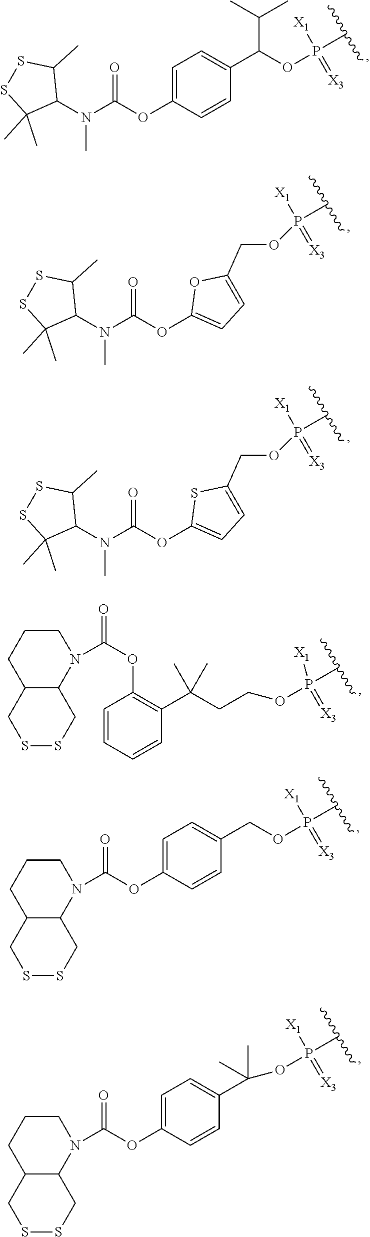

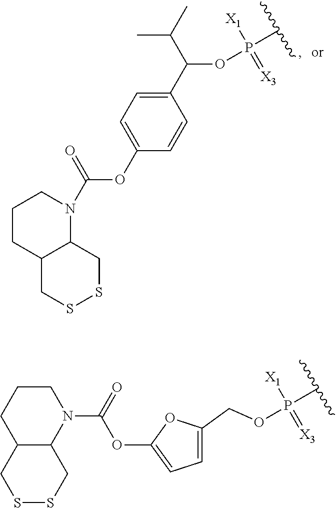

wherein,
[symbol] is selected from OH or

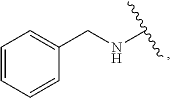 [SMILES: *NCc1ccccc1]

or represents a bond connecting to a hydroxyl or a sulfydryl of the 5′ carbon of the ribose of an adjacent nucleotide;
X₁ is selected from OH or

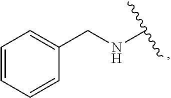 [SMILES: *NCc1ccccc1]

or is a bond connecting to a hydroxyl or a sulfydryl of the 2′ or 3′ carbon of the ribose of the other adjacent nucleotide;
and [symbol] and X₁ are not OH or

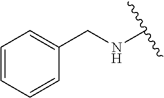 [SMILES: *NCc1ccccc1]

simultaneously;
X₃ is selected from O or S.

A31. The oligonucleotide of any one of technical solutions A16-A30, comprising 14 to 30 nucleotides.

A32. The oligonucleotide of any one of technical solutions A16-A30, comprising at its 5′ end a compound of formula (Ia), or a pharmaceutically acceptable salt, tautomer or stereoisomer thereof.

A33. The oligonucleotide of any one of technical solutions A16-A30, comprising at its 3′ end a compound of formula (Ia), or a pharmaceutically acceptable salt, tautomer or stereoisomer thereof.

A34. The oligonucleotide of any one of technical solutions A16-A30, comprising at both 5′ and 3′ ends a compound of formula (Ia), or a pharmaceutically acceptable salt, tautomer or stereoisomer thereof.

A35. The oligonucleotide of any one of technical solutions A16-A20 and A26-A30, comprising at its internal positions one or more compounds of formula (I′), or a pharmaceutically acceptable salt, tautomer or stereoisomer thereof.

A36. A dsRNA having a sense strand and an antisense strand, wherein each strand has 14-30 nucleotides; wherein the antisense strand comprise a sequence sufficiently complementary to the sense strand and target mRNA; wherein the sense strand and/or the antisense strand comprise one or more compounds of formula (Ia) of any one of technical solutions A16-A35, or a pharmaceutically acceptable salt, tautomer or stereoisomer thereof.

A37. The dsRNA of technical solution A36, wherein the sense strand comprises at its 5′ end the compound of formula (Ia) of any one of technical solutions A16-A35, or a pharmaceutically acceptable salt, tautomer or stereoisomer thereof.

A38. The dsRNA of technical solutions A36 or A37, wherein the sense strand comprises at its 3′ end the compound of formula (Ia) of any one of technical solution A16-A35, or a pharmaceutically acceptable salt, tautomer or stereoisomer thereof.

A39. The dsRNA of any one of technical solutions A36-A38, wherein the sense strand comprises at both 5′ and 3′ ends the compound of formula (Ia) of any one of technical solution A16-A35, or a pharmaceutically acceptable salt, tautomer or stereoisomer thereof.

A40. The dsRNA of any one of technical solutions A36-A39, wherein the sense strand comprises at its internal positions one or more compounds of formula (I′) in any one of technical solutions A16-A20 and A26-A30, or a pharmaceutically acceptable salt, tautomer or stereoisomer thereof.

A41. The dsRNA of any one of technical solutions A36-A40 wherein the antisense strand comprises at its 5′ end the compound of formula (Ia) of any one of technical solutions A16-A35, or a pharmaceutically acceptable salt, tautomer or stereoisomer thereof.

A42. The dsRNA of any one of technical solutions A36-A41, wherein the antisense strand comprises at its 3′ end the compound of formula (Ia) of any one of technical solutions A16-A35, or a pharmaceutically acceptable salt, tautomer or stereoisomer thereof.

A43. The dsRNA of any one of technical solutions A36-A42, wherein the antisense strand comprises at both 5′ and 3′ ends the compound of formula (Ia) of any of technical solutions A16-A35, or a pharmaceutically acceptable salt, tautomer or stereoisomer thereof.

A44. The dsRNA of any one of technical solutions A36-A43, wherein the antisense strand comprises at its internal positions one or more compounds of formula (I′) in any one of technical solutions A16-A20 and A26-A30, or a pharmaceutically acceptable salt, tautomer or stereoisomer thereof.

A45. The dsRNA of any one of technical solutions A36-A44, which is selected from small interfering RNA (siRNA) or small hairpin RNA (shRNA).

A46. A vector comprising a nucleotide sequence that encodes the dsRNA of any one of technical solutions A36-A45.

A47. A cell comprising the dsRNA of any one of technical solutions A36-A45 or the vector of technical solution A46.

A48. A pharmaceutical composition comprising the dsRNA of any one of technical solutions A36-A45, the vector of technical solution A46, or the cell of technical solution A47, and optionally a pharmaceutically acceptable carrier or excipient.

A49. A kit comprising the dsRNA of any one of technical solutions A36-A45, the vector of technical solution A46, or the cell of technical solution A47.

A50. A method of improving the efficacy of a siRNA in a cell, comprising a step of introducing the dsRNA of any one of technical solutions A36-A45 or the vector of technical solution A46 to the cell.

A51. A compound of formula (IIIb) or (IVb), or a pharmaceutically acceptable salt, tautomer or stereoisomer thereof:

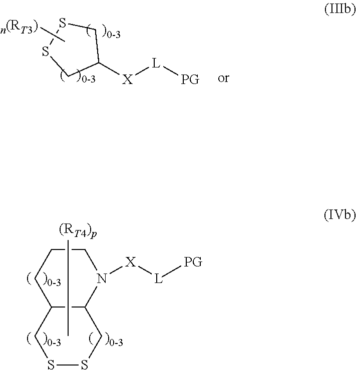

wherein,
each R_{T3} is independently selected from H, D, C₁₋₆ alkyl, C₁₋₆ haloalkyl, C₂₋₆ alkenyl or C₂₋₆ alkynyl, which is optionally deuterated or fully deuterated;
each R_{T4} is independently selected from H, D, C₁₋₆ alkyl, C₁₋₆ haloalkyl, C₂₋₆ alkenyl or C₂₋₆ alkynyl, which is optionally deuterated or fully deuterated;
n is 0, 1, 2, 3, 4 or 5;
p is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₆ alkyl or C₁₋₆ haloalkyl, which is optionally deuterated or fully deuterated;
L is —Ar—(CH₂)₁₋₆—O—, wherein each CH₂ may optionally be substituted with 1 or 2 R #; R # is selected from H, D, C₁₋₆ alkyl or C₁₋₆ haloalkyl, which is optionally deuterated or fully deuterated;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₆ alkyl, C₁₋₆ haloalkyl, C₂₋₆ alkenyl or C₂₋₆ alkynyl, which is optionally deuterated or fully deuterated;
PG is selected from a protecting group.

A52. The compound of technical solution A51, or a pharmaceutically acceptable salt, tautomer or stereoisomer thereof, wherein,
each R_{T3} is independently selected from H, D, C₁₋₆ alkyl or C₁₋₆ haloalkyl, which is optionally deuterated or fully deuterated;
each R_{T4} is independently selected from H, D, C₁₋₆ alkyl or C₁₋₆ haloalkyl, which is optionally deuterated or fully deuterated;
n is 0, 1, 2 or 3;
p is 0, 1, 2 or 3;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is —Ar—(CH₂)₁₋₄—O—, wherein each CH₂ may optionally be substituted with 1 or 2 R #; R # is selected from H, D, C₁₋₄ alkyl or C₁₋₄ haloalkyl, which is optionally deuterated or fully deuterated;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₄ alkyl or C₁₋₄ haloalkyl, which is optionally deuterated or fully deuterated;
PG is selected from a protecting group.

A53. The compound of technical solutions A51 or A52, or a pharmaceutically acceptable salt, tautomer or stereoisomer thereof, wherein PG is selected from a hydroxyl protecting group, e.g., trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), p-methoxybenzyloxymethyl (PMBM), —C(O)CH₂CH₂C(O)OH or 4,4′-dimethoxytrityl, preferably —C(O)CH₂CH₂C(O)OH or 4,4′-dimethoxytrityl, more preferably —C(O)CH₂CH₂C(O)OH.

A54. The compound of any one of technical solutions A51-A5, or a pharmaceutically acceptable salt, tautomer or stereoisomer thereof, wherein the compound of formula (IIIb) or (IVb) has a structure of formula (III-1b) or (IV-1b),

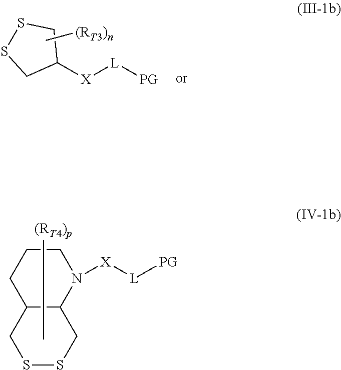

wherein,
each R_{T3} is independently selected from H, D, C₁₋₄ alkyl or C₁₋₄ haloalkyl, which is optionally deuterated or fully deuterated;
each R_{T4} is independently selected from H, D, C₁₋₄ alkyl or C₁₋₄ haloalkyl, which is optionally deuterated or fully deuterated;
n is 0, 1, 2 or 3;
p is 0, 1, 2 or 3;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is selected from

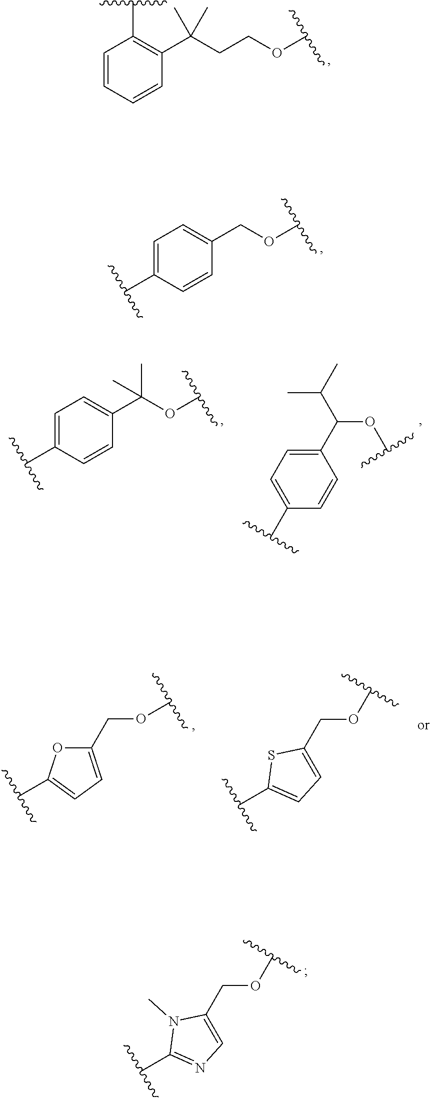

PG is a protecting group selected from DMTr, —P(OCH₂CH₂CN)(N(iPr)₂) or —C(O)CH₂CH₂C(O)OH.

A55. The compound of technical solution A54, or a pharmaceutically acceptable salt, tautomer or stereoisomer thereof, wherein,
R_{T3} is selected from H, D or CH₃, which is optionally deuterated or fully deuterated;
R_{T4} is selected from H or D;
n is 3;
p is 0;
X is selected from bond —O—, —NHC(O)O—, —OC(O)NH—, —N(CH₃)C(O)O— or —C(O)O—;
L is selected from

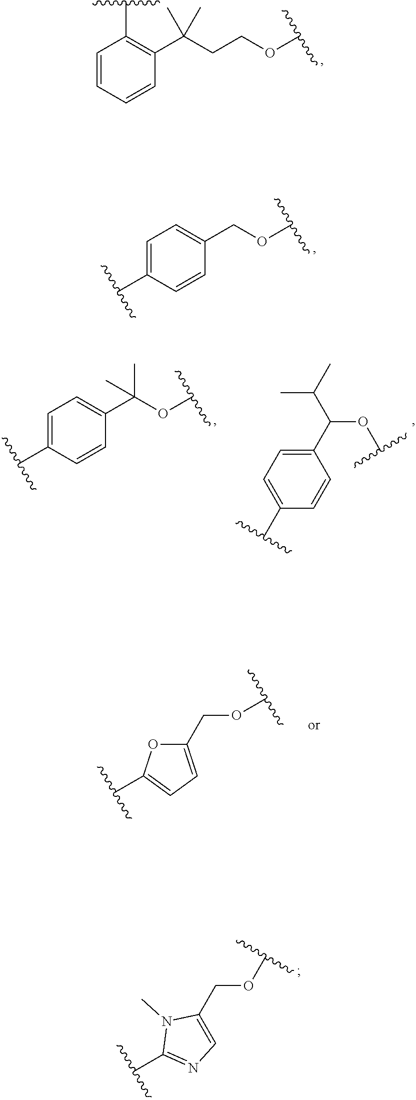

PG is selected from DMTr, —P(OCH₂CH₂CN)(N(iPr)₂) or —C(O)CH₂CH₂C(O)OH.

A56. The compound of any one of technical solutions A51-A55, or a pharmaceutically acceptable salt, tautomer or stereoisomer thereof, wherein the compound of formula (IIIb) or (IVb) is selected from:

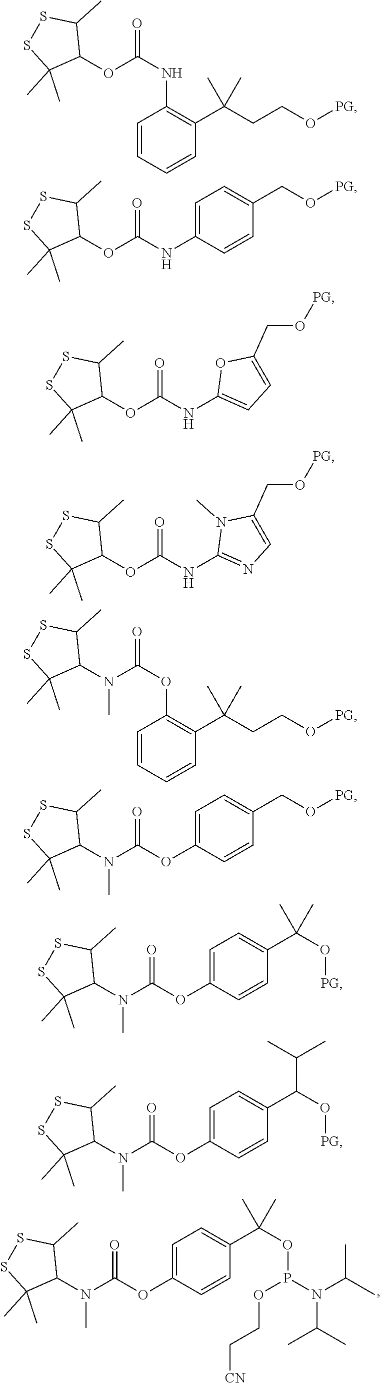

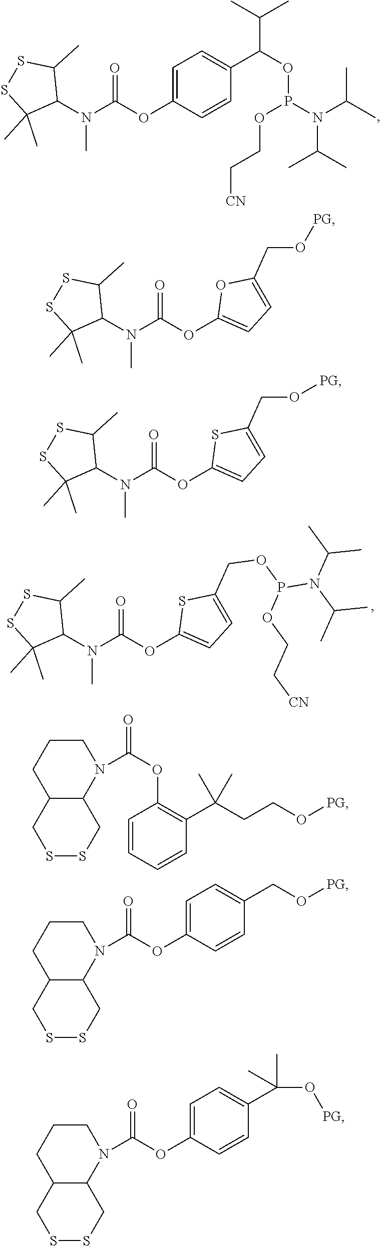

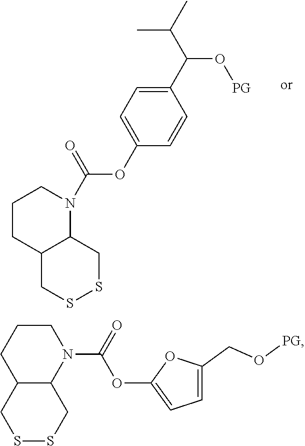

wherein,
PG is selected from a protecting group, preferably a hydroxyl protecting group, e.g., trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), p-methoxybenzyloxymethyl (PMBM), —C(O)CH₂CH₂C(O)OH or 4,4′-dimethoxytrityl, preferably —C(O)CH₂CH₂C(O)OH or 4,4′-dimethoxytrityl, more preferably —C(O)CH₂CH₂C(O)OH.

A57. The compound of formula (I) of any one of technical solutions A1-A3 and A6-A8 or the oligonucleotide of any one of technical solutions A16-18 and A21-23, wherein the GalNAc-containing chain is a conjugated group of formula (X′):

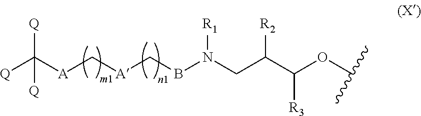

wherein,
[symbol] represents the position of attachment to biomolecules;
Q is independently H,

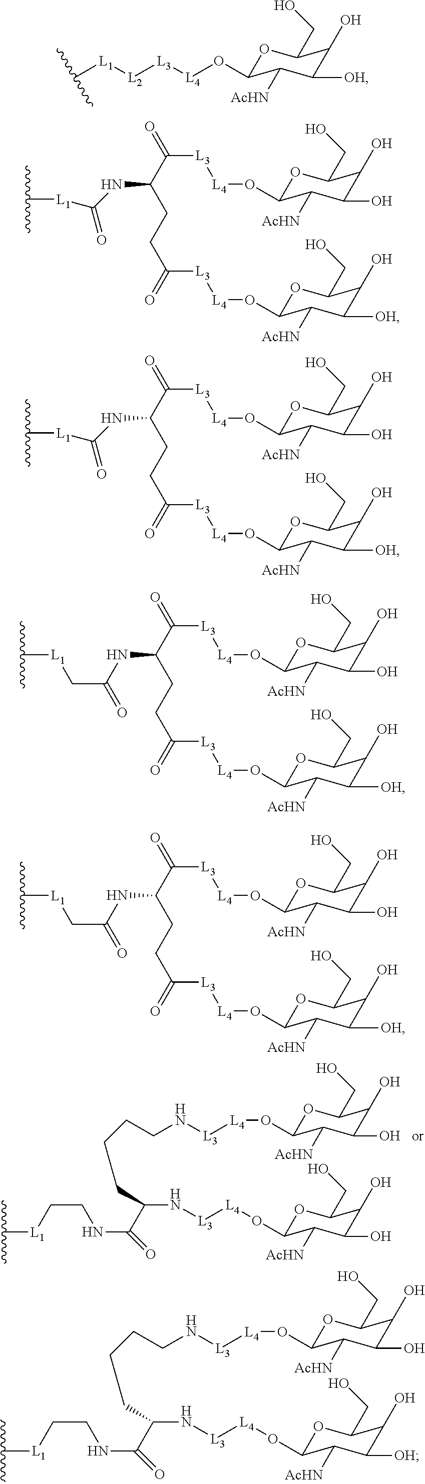

wherein L₁ is bond, —CH₂—, —CH₂CH₂—, —C(O)—, —CH₂O—, —CH₂O—CH₂CH₂O— or —NHC(O)—(CH₂NHC(O))ₐ—;
L₂ is bond or —CH₂CH₂C(O)—;
L₃ is bond, —(NHCH₂CH₂)_{b}—, —(NHCH₂CH₂CH₂)_{b}— or —C(O)CH₂—;
L₄ is —(OCH₂CH₂)ₑ—, —(OCH₂CH₂CH₂)ₑ—, —(OCH₂CH₂CH₂CH₂)ₑ—, —(OCH₂CH₂CH₂CH₂CH₂)ₑ— or —NHC(O)—(CH₂)ₐ—;
   wherein a=0, 1, 2 or 3;
   b=1, 2, 3, 4 or 5;
   c=1, 2, 3, 4 or 5;
   d=1, 2, 3, 4, 5, 6, 7 or 8;
A is bond, —CH₂O— or —NHC(O)—;
A′ is bond, —C(O)NH—, —NHC(O)— or —O(CH₂CH₂O)ₑ—;
   wherein e is 1, 2, 3, 4 or 5;
B is bond, —CH₂—, -Q(O)—, -M-, —CH₂-M- or —C(O)-M-;
   wherein M is

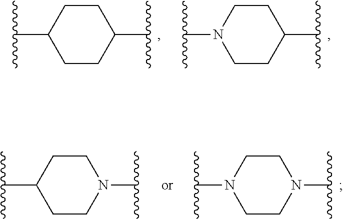

R₁ and R₂ together form —CH₂CH₂O— or —CH₂CH(R)—O—; and R₃ is H;
or R₁ and R₃ together form —C₁₋₂ alkylene-; and R₂ is H;
   wherein R is —OR′, —CH₂OR′ or —CH₂CH₂OR′, wherein R′ is H, a hydroxy-protecting group, or a solid support, the hydroxy-protecting group is preferably —C(O)CH₂CH₂C(O)OH or 4,4′-dimethoxytrityl;
   m1=0, 1, 2, 3, 4, 5, 6, 7, 8, 9 or 10;
   n1=0, 1, 2, 3, 4, 5, 6, 7, 8, 9 or 10.

The present invention also relates to the following technical solutions:

B1. An oligonucleotide comprising a compound of formula (II), or a pharmaceutically acceptable salt, tautomer or stereoisomer thereof:

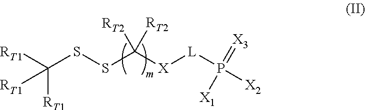

wherein,
X₁ is selected from ORₐ or NR_{b}R_{c};
Rₐ is selected from H, C₁₋₆ alkyl or C₁₋₆ haloalkyl, which is optionally deuterated or fully deuterated;
R_{b} and R_{c} are selected from H, C₁₋₆ alkyl or C₁₋₆ haloalkyl; R_{b} and R_{c} may optionally be substituted with D, C₆₋₁₀ aryl or 5-10-membered heteroaryl, or fully deuterated;
X₂ is the remainder of the oligonucleotide, which connects to P via a hydroxyl or a sulfydryl on the 2′, 3′ or 5′ carbon of the ribose of its first nucleotide counting from the 5′ end;
X₃ is independently selected from O or S;
each R_{T1} is independently selected from H, D, C₁₋₆ alkyl, C₁₋₆ haloalkyl, C₂₋₆ alkenyl, C₂₋₆ alkynyl or GalNAc-containing chain, which is optionally deuterated or fully deuterated;
each R_{T2} is independently selected from H, D, C₁₋₆ alkyl, C₁₋₆ haloalkyl, C₂₋₆ alkenyl or C₂₋₆ alkynyl, which is optionally deuterated or fully deuterated;
m is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₆ alkyl or C₁₋₆ haloalkyl, which is optionally deuterated or fully deuterated;
L is —Ar—(CH₂)₁₋₆—O—, wherein each CH₂ may optionally be substituted with 1 or 2 R #; R # is selected from H, D, C₁₋₆ alkyl or C₁₋₆ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2, 3, 4 or 5 R*;
R* is selected from C₁₋₆ alkyl, C₁₋₆ haloalkyl, C₂₋₆ alkenyl or C₂₋₆ alkynyl, which is optionally deuterated or fully deuterated.

B2. The oligonucleotide of technical solution B1, or a pharmaceutically acceptable salt, tautomer or stereoisomer thereof, wherein,
X₁ is selected from ORₐ or NR_{b}R_{c};
Rₐ is selected from H, C₁₋₄ alkyl or C₁₋₄ haloalkyl, which is optionally deuterated or fully deuterated;
R_{b} and R_{c} are independently selected from H, C₁₋₄ alkyl, C₁₋₄ haloalkyl; R_{b} and R_{c} may optionally be substituted with D, C₆₋₁₀ aryl, or fully deuterated;
X₂ is the remainder of the oligonucleotide, which connects to P via a hydroxyl or a sulfydryl on the 5′ carbon of the ribose of its first nucleotide counting from the 5′ end;
X₃ is independently selected from O or S;
each R_{T1} is independently selected from H, D, C₁₋₄ alkyl, C₁₋₄ haloalkyl or GalNAc-containing chain, which is optionally deuterated or fully deuterated;
each R_{T2} is independently selected from H, D, C₁₋₄ alkyl or C₁₋₄ haloalkyl, which is optionally deuterated or fully deuterated;
m is 0, 1, 2 or 3;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is —Ar—(CH₂)—O—, wherein CH₂ may optionally be substituted with 1 or 2 R #; R # is selected from H, D, C₁₋₄ alkyl or C₁₋₄ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₄ alkyl or C₁₋₄ haloalkyl, which is optionally deuterated or fully deuterated.

B3. The oligonucleotide of technical solutions B1 or B2, or a pharmaceutically acceptable salt, tautomer or stereoisomer thereof, wherein,
X₁ is selected from ORₐ or NR_{b}R_{c};
Rₐ is selected from H, C₁₋₄ alkyl or C₁₋₄ haloalkyl, which is optionally deuterated or fully deuterated;
R_{b} and R_{c} are independently selected from H, C₁₋₄ alkyl, C₁₋₄ haloalkyl; R_{b} and R_{c} are optionally substituted with D, phenyl, or fully deuterated;
X₂ is the remainder of the oligonucleotide, which connects to P via a hydroxyl or a sulfydryl on the 5′ carbon of the ribose of its first nucleotide counting from the 5′ end;
X₃ is independently selected from O or S;

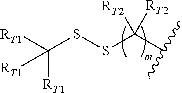

is selected from

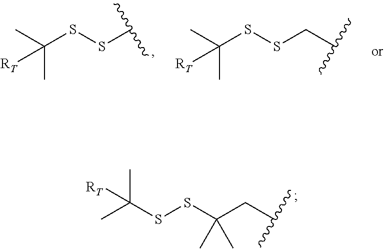

wherein R_{T} is selected from H, D, CH₃ or GalNAc-containing chain, which is optionally deuterated or fully deuterated;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is selected from

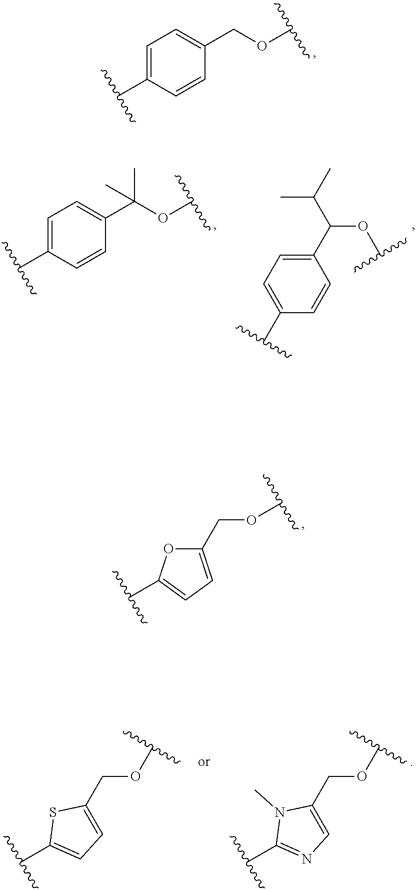

B4. The oligonucleotide of any one of technical solution B1-B3, or a pharmaceutically acceptable salt, tautomer or stereoisomer thereof wherein,
X₁ is selected from OH or

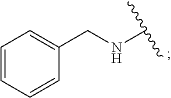 [SMILES: *NCc1ccccc1]

X₂ is the remainder of the oligonucleotide, which connects to P via a hydroxyl or a sulfydryl on the 5′ carbon of the ribose of its first nucleotide counting from the 5′ end;
X₃ is independently selected from O or S;

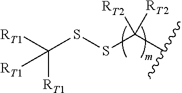

is selected from

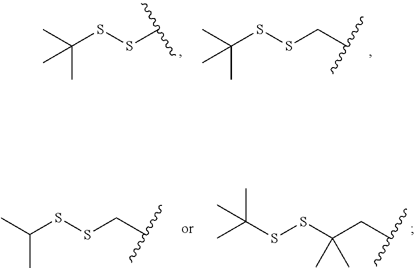

X is selected from bond, —O—, —NHC(O)O—, —OC(O)NH—, —N(CH₃)C(O)O— or —C(O)O—;
L is selected from

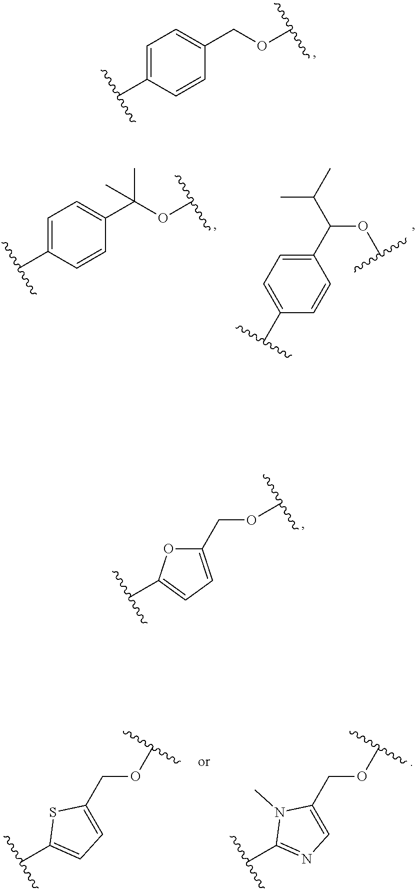

B5. The oligonucleotide of any one of technical solutions B1-B4, or a pharmaceutically acceptable salt, tautomer or stereoisomer thereof, wherein the compound of formula (II) is selected from:

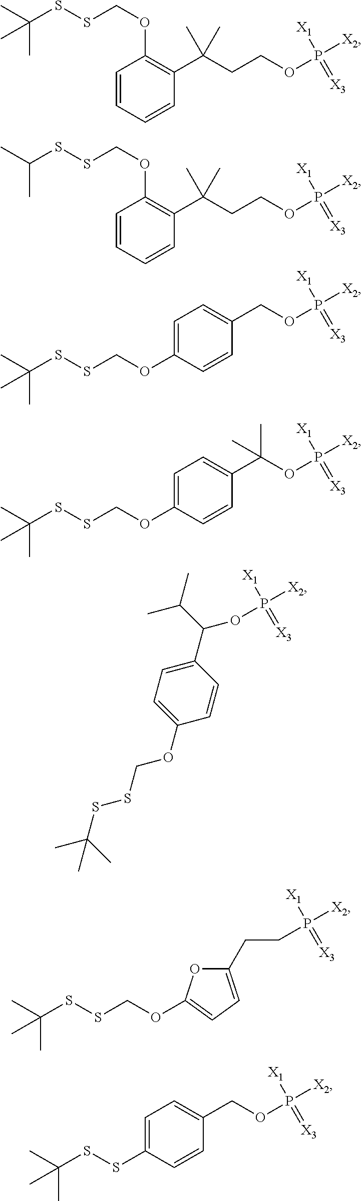

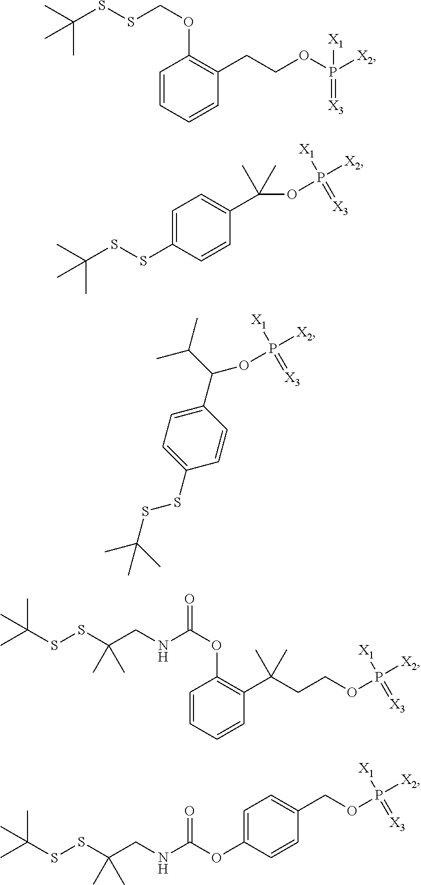

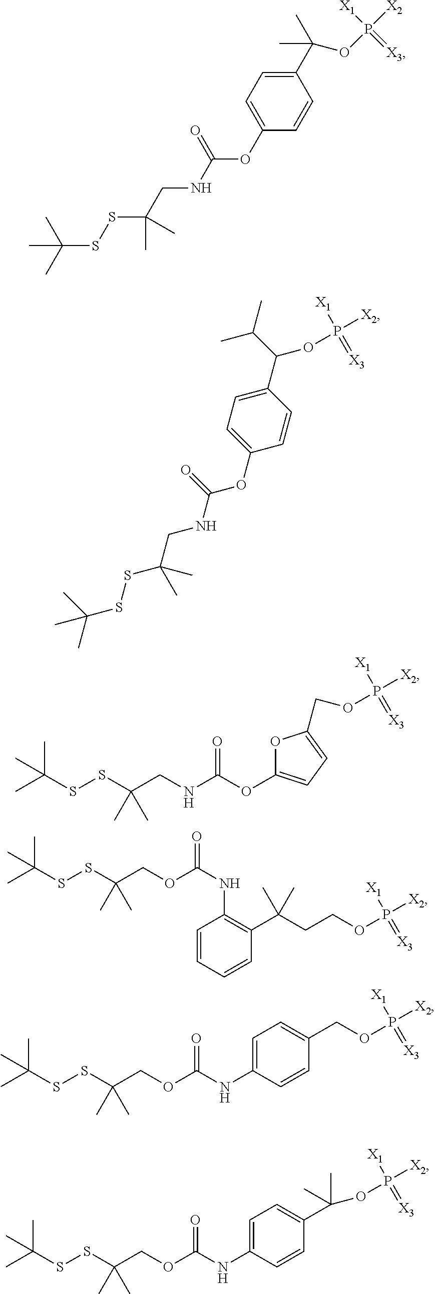

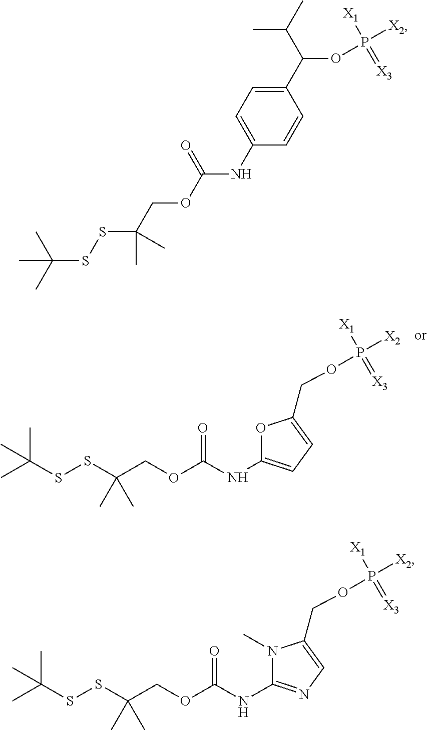

wherein
X₁ is selected from OH or

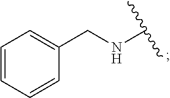 [SMILES: *NCc1ccccc1]

X₂ is the remainder of the oligonucleotide, which connects to P via a hydroxyl or a sulfydryl on the 5′ carbon of the ribose of its first nucleotide counting from the 5′ end;
X₃ is independently selected from O or S;
preferably, the compound of formula (I) is selected from:

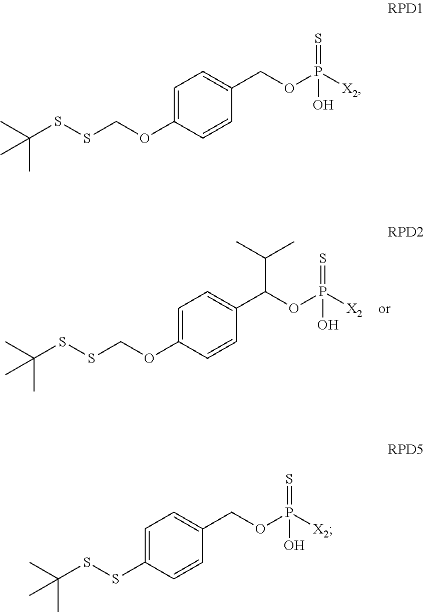

X₂ is the remainder of the oligonucleotide, which connects to P via a hydroxyl or a sulfydryl on the 5′ carbon of the ribose its first nucleotide counting from the 5′ end.

B6. An oligonucleotide comprising one, two or more compounds of formula (Ia), or a pharmaceutically acceptable salt, tautomer or stereoisomer thereof:

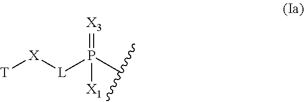

wherein,
[symbol] is selected from ORₐ or NR_{b}R_{c}, or represents a bond connecting to a hydroxyl or a sulfydryl of the 5′ carbon of the ribose of an adjacent nucleotide;
X₁ is a bond connecting to a hydroxyl or a sulfydryl of the 2′ or 3′ carbon of the ribose of the other adjacent nucleotide;
Rₐ is selected from H, C₁₋₆ alkyl, C₁₋₆ haloalkyl, C₂₋₆ alkenyl or C₂₋₆ alkynyl, which is optionally deuterated or fully deuterated;
R_{b} and R_{c} is independently selected from H, C₁₋₆ alkyl or C₁₋₆ haloalkyl; R_{b} and R_{c} may be optionally substituted with D, C₆₋₁₀ aryl or 5-10-membered heteroaryl, or fully deuterated;
X₃ is independently selected from O or S;
T is

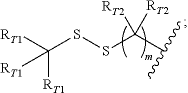

each R_{T1} is independently selected from H, D, halogen, CN, C₁₋₆ alkyl, C₁₋₆ haloalkyl, C₁₋₆ haloalkyl, C₂₋₆ alkenyl, C₂₋₆ alkynyl or GalNAc-containing chain, which is optionally deuterated or fully deuterated;
each R_{T2} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
m is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, halogen, C₁₋₆ alkyl or C₁₋₆ haloalkyl, which is optionally deuterated or fully deuterated;
L is —Ar—(CH₂)₁₋₆—O—, wherein each CH₂ may optionally be substituted with R #; R # is selected from H, D, halogen, CN, C₁₋₆ alkyl, C₁₋₆ haloalkyl, C₂₋₆ alkenyl or C₂₋₆ alkynyl, which is optionally deuterated or fully deuterated;
Ar in L is linked to X; the oxygen atom in L is linked to the phosphorus atom;
Ar is selected from C₃₋₁₀ cycloalkyl, 3-10-membered heterocyclyl, C₆₋₁₀ aryl or 5-14-membered heteroaryl; C₃₋₁₀ cycloalkyl, 3-10-membered heterocyclyl, C₆₋₁₀ aryl or 5-10-membered heteroaryl may optionally be substituted with 1, 2, 3, 4 or 5 R*;
R* is selected from H, D, halogen, CN, C₁₋₆ alkyl, C₁₋₆ haloalkyl, C₂₋₆ alkenyl or C₂₋₆ alkynyl, which is optionally deuterated or fully deuterated.

B7. The oligonucleotide of technical solution B6, wherein,
[symbol] is selected from ORₐ or NR_{b}R_{c}, or represents a bond connecting to a hydroxyl or a sulfydryl of the 5′ carbon of the ribose of an adjacent nucleotide;
X₁ is a bond connecting to a hydroxyl or a sulfydryl of the 2′ or 3′ carbon of the ribose of the other adjacent nucleotide;
Rₐ is selected from H, C₁₋₄ alkyl or C₁₋₄ haloalkyl, which is optionally deuterated or fully deuterated;
R_{b} and R_{c} are independently selected from H, C₁₋₄ alkyl or C₁₋₄ haloalkyl; and R_{b} and R_{c} may optionally be substituted with D, C₆₋₁₀ aryl, which is optionally deuterated or fully deuterated;
X₂ and X₃ is independently selected from O or S;
T is selected from

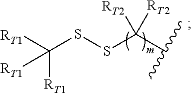

each R_{T1} is independently selected from H, D, C₁₋₄ alkyl, C₁₋₄ haloalkyl or GalNAc-containing chain, which is optionally deuterated or fully deuterated;
each R_{T2} is independently selected from H, D, C₁₋₄ alkyl or C₁₋₄ haloalkyl, which is optionally deuterated or fully deuterated;
m is 0, 1, 2 or 3;
X is selected from bond, —O—, —S—, —OC(O)NR_{X1}—, —NR_{X1}C(O)O—, —C(O)O—, —OC(O)—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is —Ar—(CH₂)—O—, wherein CH₂ may optionally be substituted with 1 or 2 R #; R # is selected from H, D, C₁₋₄ alkyl or C₁₋₄ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₄ alkyl or C₁₋₄ haloalkyl, which is optionally deuterated or fully deuterated.

B8. The oligonucleotide of technical solutions B6 or B7, wherein,
[symbol] is selected from ORₐ or NR_{b}R_{c}, or represents a bond connecting to a hydroxyl or a sulfydryl of the 5′ carbon of the ribose of an adjacent nucleotide;
X₁ is a bond connecting to a hydroxyl or a sulfydryl of the 2′ or 3′ carbon of the ribose of the other adjacent nucleotide;
Rₐ is selected from H, C₁₋₄ alkyl or C₁₋₄ haloalkyl, which is optionally deuterated or fully deuterated;
R_{b} and R_{c} are independently selected from H, C₁₋₄ alkyl or C₁₋₄ haloalkyl; R_{b} and R_{c} are optionally substituted with D, phenyl, or fully deuterated;
X₃ is independently selected from O or S;
T is selected from

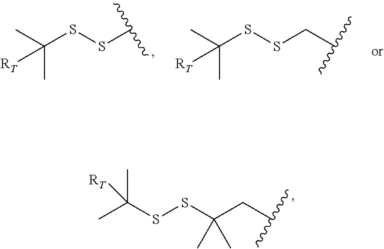

wherein R_{T} is selected from H, D, CH₃ or GalNAc-containing chain, which is optionally deuterated or fully deuterated;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is selected from

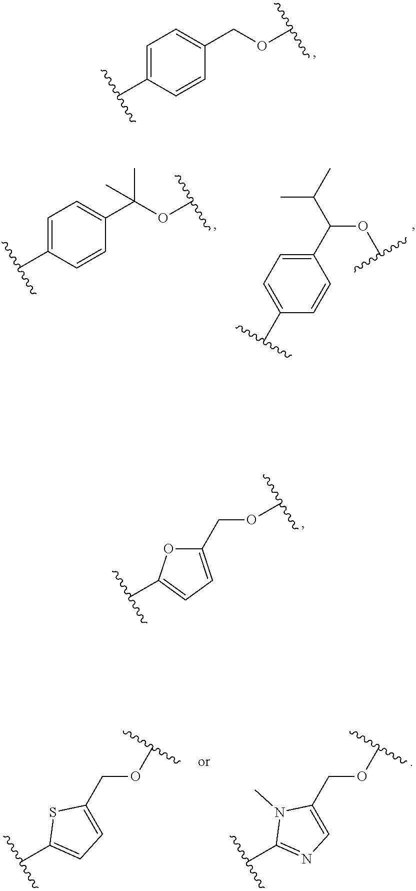

B9. The oligonucleotide of any one of technical solutions B6-B8, wherein,
[symbol] represents a bond connecting to a hydroxyl or a sulfydryl of the 5′ carbon of the ribose of an adjacent nucleotide;
X₁ is a bond connecting to a hydroxyl or a sulfydryl of the 2′ or 3′ carbon of the ribose of the other adjacent nucleotide;
X₃ is selected from O or S;
T is selected from

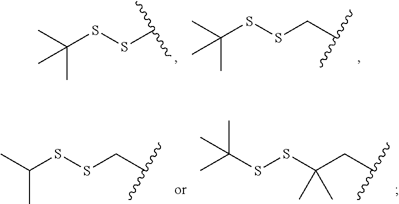

X is selected from bond, —O—, —NHC(O)O—, —OC(O)NH—, —N(CH₃)C(O)O— or —C(O)O—;
L is selected from

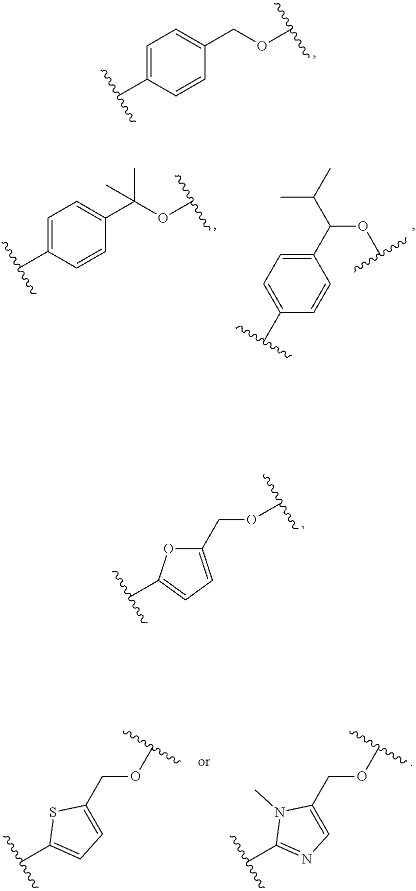

B10. The oligonucleotide of any one of technical solutions B6-B9 oligonucleotide, wherein, the compound of formula (Ia) is selected from the following structures:

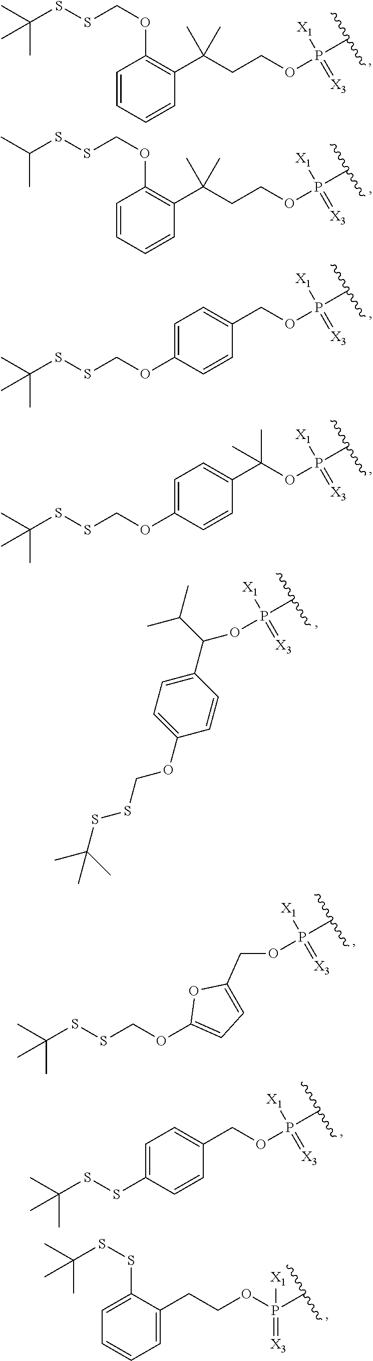

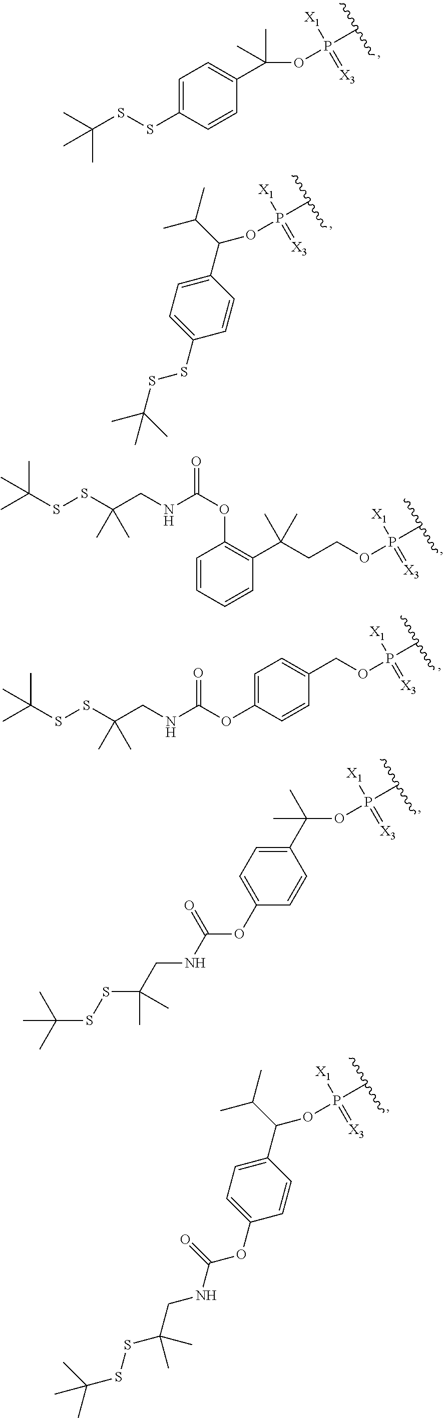

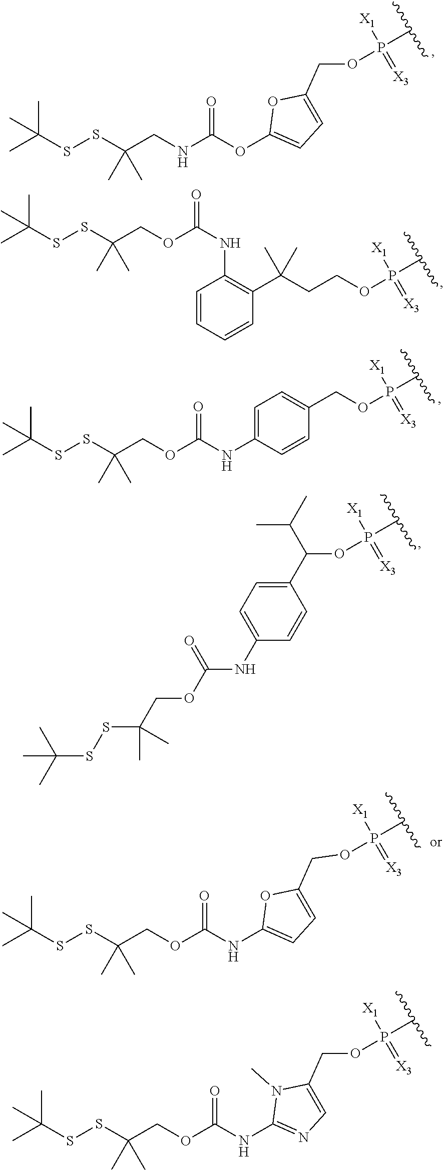

wherein
[symbol] represents a bond connecting to a hydroxyl or a sulfydryl of the 5′ carbon of the ribose of an adjacent nucleotide;
X¹ is a bond connecting to a hydroxyl or a sulfydryl of the 2′ or 3′ carbon of the ribose of the other adjacent nucleotide;
X₃ is selected from O or S;
preferably, the compound of formula (Ia) is selected from:

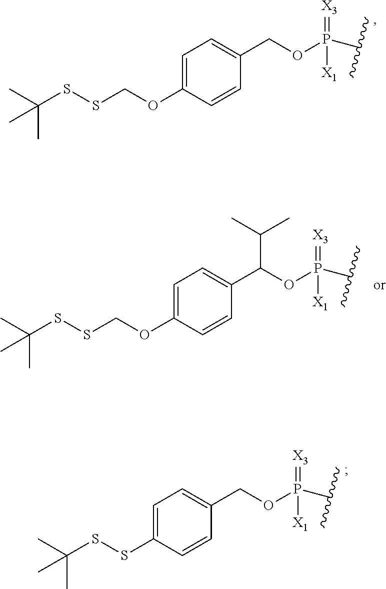

[symbol] represents a bond connecting to a hydroxyl of the 5′ carbon of the ribose of an adjacent nucleotide;
X₁ is a bond connecting to a hydroxyl or a sulfydryl of the 2′ or 3′ carbon of the ribose of the other adjacent nucleotide;
X₃ is selected from O or S.

B11. The oligonucleotide of any one of technical solutions B6-B10, having 14 to 30 nucleotides.

B12. The oligonucleotide of any one of technical solutions B6-B11, comprising one or more compounds of formula (I) in the internal positions of the oligonucleotide, or a pharmaceutically acceptable salt, tautomer or stereoisomer thereof.

B13. An dsRNA having a sense strand and an antisense strand, wherein each strand has 14-30 nucleotides; wherein the antisense strand comprise a sequence sufficiently complementary to the sense strand and target mRNA; wherein the sense strand and/or the antisense strand comprise one or more compounds of formula (II) of any one of technical solutions B1-B5 or one or more compounds of formula (Ia) of any one of technical solutions B6-B10, or a pharmaceutically acceptable salt, tautomer or stereoisomer thereof.

B14. The dsRNA of technical solution B13, wherein the sense strand comprises in its internal position one or more compounds of formula (Ia) of any one of technical solutions B6-B10, or a pharmaceutically acceptable salt, tautomer or stereoisomer thereof.

B15. The dsRNA of technical solution B13, wherein the antisense strand comprises at its 5′ end one compound of formula (II) of any one of technical solutions B1-B5, or a pharmaceutically acceptable salt, tautomer or stereoisomer thereof.

B16. The dsRNA of technical solution B13, wherein the antisense strand comprises in its internal positions one or more compounds of formula (Ia) of any one of technical solutions B6-B10, or a pharmaceutically acceptable salt, tautomer or stereoisomer thereof.

B17. The dsRNA of any one of technical solutions B13-B16, which is selected from small interfering RNA (siRNA) or small hairpin RNA (shRNA).

B18. A cell comprising the dsRNA of any one of technical solutions B14-B17.

B19. A pharmaceutical composition comprising the dsRNA of any one of technical solutions B14-B17, or the cell of technical solution B18, and optionally a pharmaceutically acceptable carrier or excipient.

B20. A kit comprising the dsRNA of any one of technical solutions B14-B17, the cell of technical solution B18, or the pharmaceutical composition of technical solution B19.

B21. A compound of formula (IIb), or a pharmaceutically acceptable salt, tautomer or stereoisomer thereof,

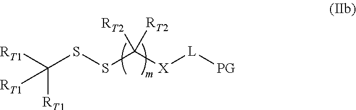

wherein,
each R_{T1} is independently selected from H, D, C₁₋₆ alkyl, C₁₋₆ haloalkyl, C₂₋₆ alkenyl, C₂₋₆ alkynyl or GalNAc-containing chain, which is optionally deuterated or fully deuterated;
each R_{T2} is independently selected from H, D, C₁₋₆ alkyl, C₁₋₆ haloalkyl, C₂₋₆ alkenyl or C₂₋₆ alkynyl, which is optionally deuterated or fully deuterated;
m is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₆ alkyl or C₁₋₆ haloalkyl, which is optionally deuterated or fully deuterated;
L is —Ar—(CH₂)—O—, wherein CH₂ may optionally be substituted with 1 or 2 R #; R # is selected from H, D, C₁₋₆ alkyl or C₁₋₆ haloalkyl, which is optionally deuterated or fully deuterated;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₆ alkyl, C₁₋₆ haloalkyl, C₂₋₆ alkenyl or C₂₋₆ alkynyl, which is optionally deuterated or fully deuterated;
PG is selected from a protecting group.

B22. The compound of technical solution B21, or a pharmaceutically acceptable salt, tautomer or stereoisomer thereof, wherein,
each R_{T1} is independently selected from H, D, C₁₋₆ alkyl, C₁₋₆ haloalkyl or GalNAc-containing chain, which is optionally deuterated or fully deuterated;
each R_{T2} is independently selected from H, D, C₁₋₆ alkyl, C₁₋₆ haloalkyl, which is optionally deuterated or fully deuterated;
m is 0, 1, 2 or 3;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is —Ar—(CH₂)—O—, wherein CH₂ may optionally be substituted with 1 or 2 R #; R # is selected from H, D, C₁₋₄ alkyl or C₁₋₄ haloalkyl, which is optionally deuterated or fully deuterated;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₄ alkyl or C₁₋₄ haloalkyl, which is optionally deuterated or fully deuterated;
PG is selected from a protecting group.

B23. The compound of technical solutions B21 or B22, or a pharmaceutically acceptable salt, tautomer or stereoisomer thereof, wherein PG is selected from a hydroxyl protecting group, e.g., trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), p-methoxybenzyloxymethyl (PMBM), —C(O)CH₂CH₂C(O)OH or 4,4′-dimethoxytrityl, preferably —C(O)CH₂CH₂C(O)OH or 4,4′-dimethoxytrityl, more preferably —C(O)CH₂CH₂C(O)OH.

B24. The compound of any one of technical solutions B21-B23, or a pharmaceutically acceptable salt, tautomer or stereoisomer thereof, wherein the compound of formula (IIb) is selected from

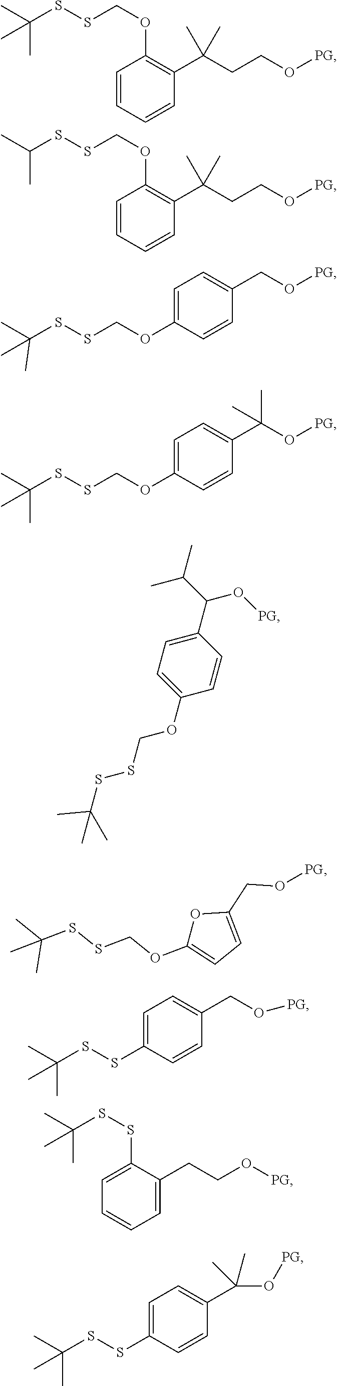

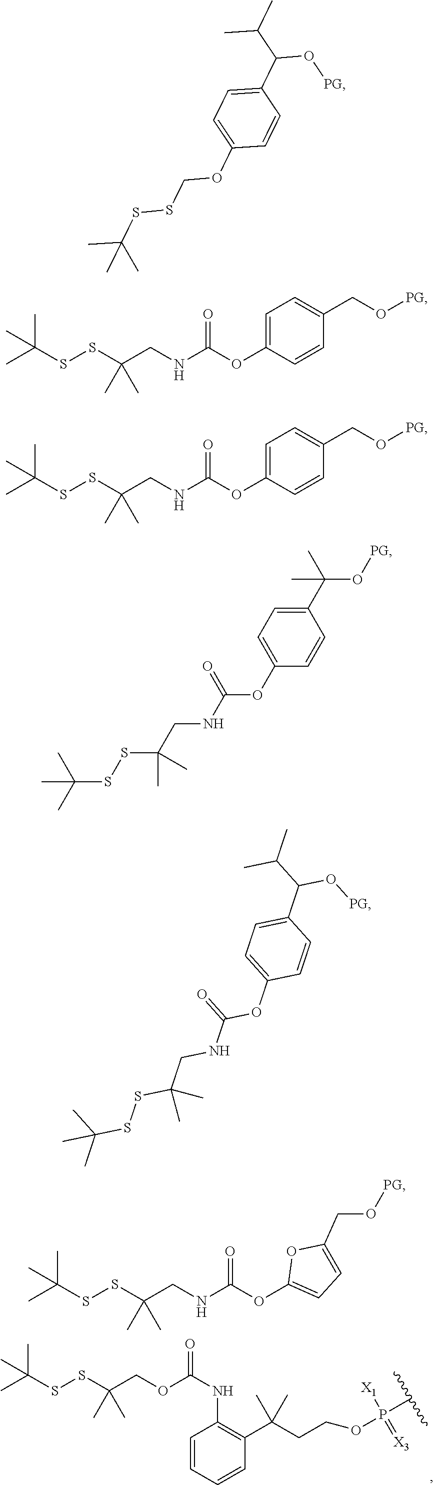

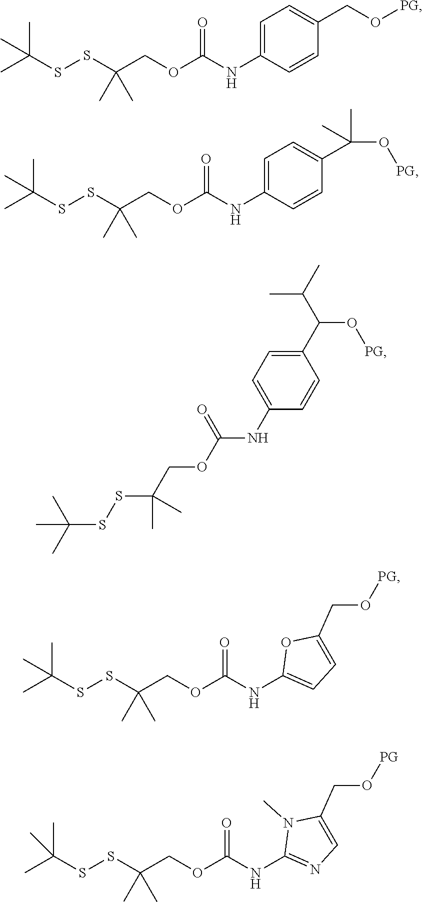

wherein,
PG is —P(OCH₂CH₂CN)(N(iPr)₂) or H.

B25. The oligonucleotide of any one of technical solutions B1-B3 and B6-B8 or the compound of formula (IIb) of technical solutions B21 or B22, wherein the GalNAc-containing chain is a conjugated group of formula (X′):

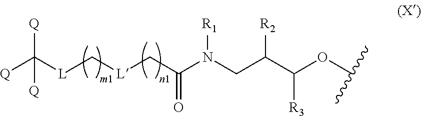

wherein,
[symbol] represents the position of attachment to biomolecules
Q is independently H,

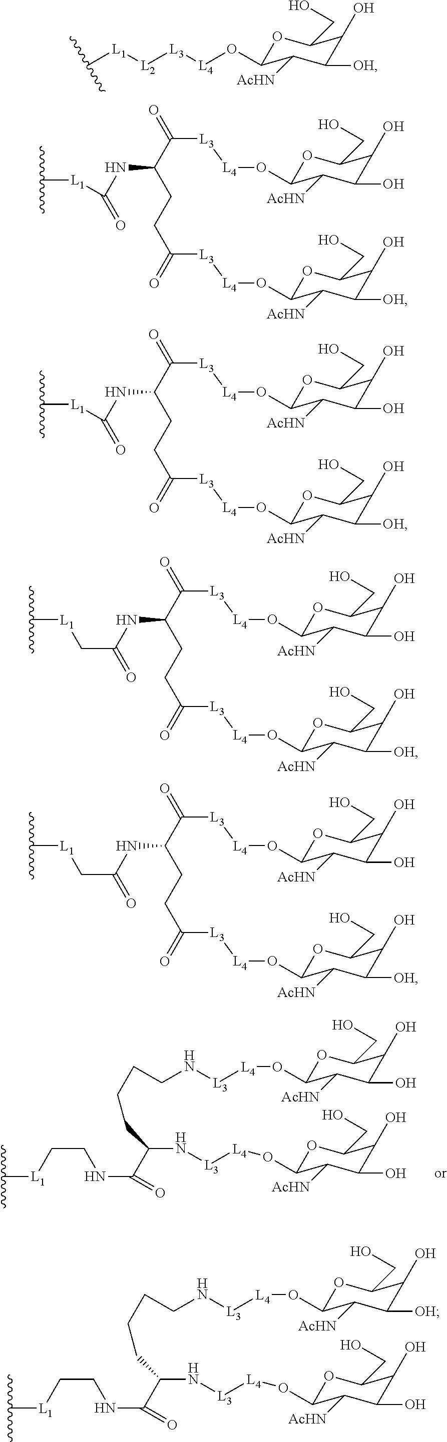

wherein L₁ is bond, —CH₂—, —CH₂CH₂—, —C(O)—, —CH₂O—, —CH₂O—CH₂CH₂O— or —NHC(O)—(CH₂NHC(O))ₐ—;
L₂ is bond or —CH₂CH₂C(O)—;
L₃ is bond, —(NHCH₂CH₂)_{b}—, —(NHCH₂CH₂CH₂)_{b}— or —C(O)CH₂—;
L₄ is —(OCH₂CH₂)ₑ—, —(OCH₂CH₂CH₂)ₑ—, —(OCH₂CH₂CH₂CH₂)ₑ—, —(OCH₂CH₂CH₂CH₂CH₂)ₑ— or —NHC(O)—(CH₂)ₐ—;
   wherein a=0, 1, 2 or 3;
   b=1, 2, 3, 4 or 5;
   c=1, 2, 3, 4 or 5;
   d=1, 2, 3, 4, 5, 6, 7 or 8;
A is bond, —CH₂O— or —NHC(O)—;
A′ is bond, —C(O)NH—, —NHC(O)— or —O(CH₂CH₂O)ₑ—;
   wherein e is 1, 2, 3, 4 or 5;
B is bond, —CH₂—, —C(O)—, -M-, —CH₂-M- or —C(O)-M-;
   wherein M is

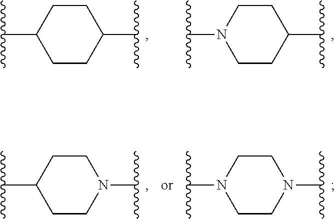 [SMILES: *C1CCC(*)CC1 ; *C1CCN(*)CC1 ; *C1CCN(*)CC1 ; *N1CCN(*)CC1]

R₁ and R₂ together form —CH₂CH₂O— or —CH₂CH(R)—O—; and R₃ is H;
or R₁ and R₃ together form —C₁₋₂ alkylene-; and R₂ is H;
   wherein R is —OR′, —CH₂OR′ or —CH₂CH₂OR′, wherein R′ is H, a hydroxy-protecting group, or a solid support, the hydroxy-protecting group is preferably —C(O)CH₂CH₂C(O)OH or 4,4′-dimethoxytrityl;
   m1=0, 1, 2, 3, 4, 5, 6, 7, 8, 9 or 10;
   n1=0, 1, 2, 3, 4, 5, 6, 7, 8, 9 or 10.

### Examples

The following examples are intended to illustrate the invention and do not limit the scope of the invention.

Abbreviations

| | |
|---|---|
| DCI | 4,5-dicyanoimidazole |
| DCM | dichloromethane |
| DMAP | 4-dimethylaminopyridine |
| DMF | N,N-dimethylformamide |
| p-TsSK | Potassium 4-methylbenzenethiosulfonate |
| TBAF | tetrabutylammonium fluoride |
| TEAB | Triethylamine bicarbonate buffer |

### Example 1 The Preparation of DN0110

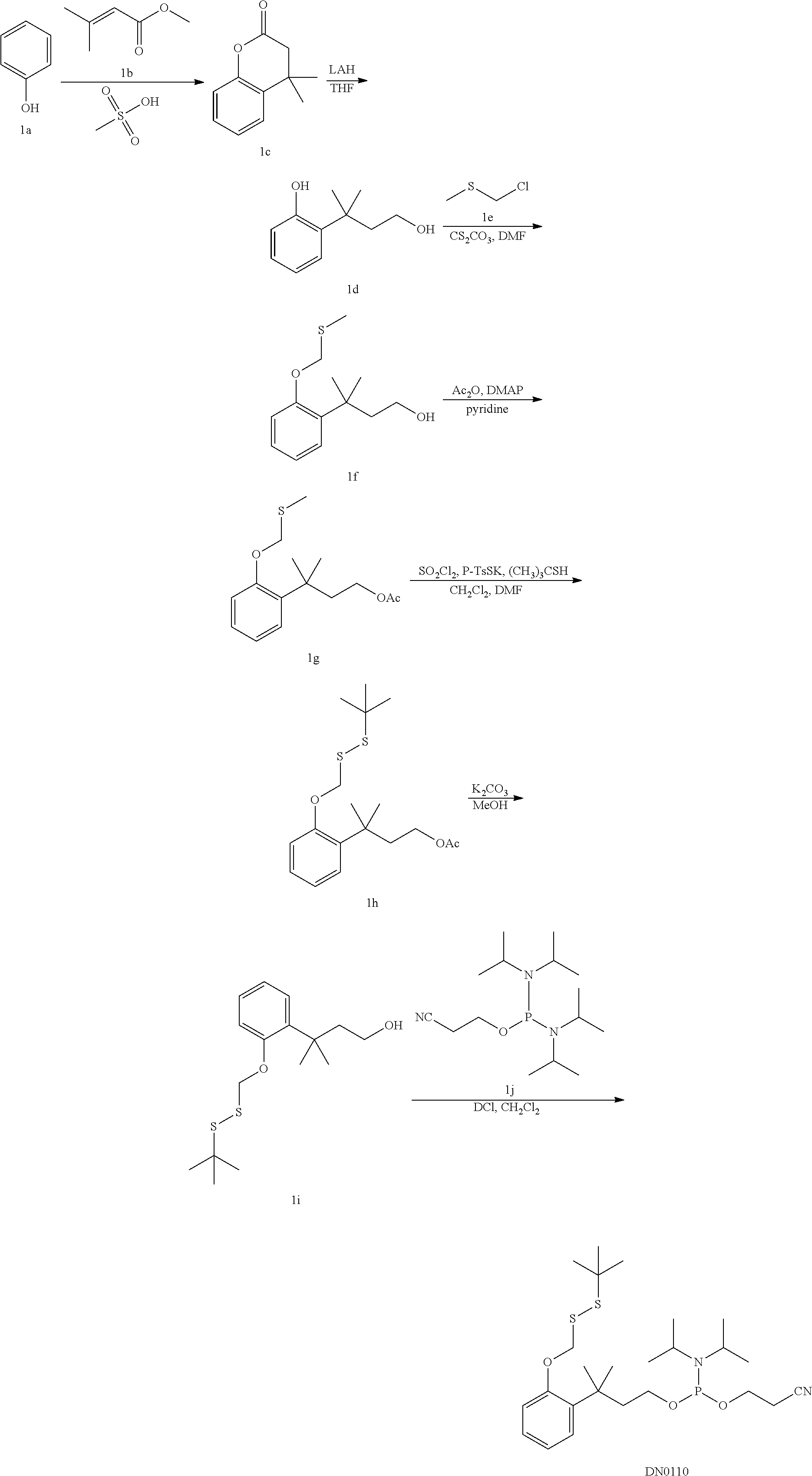

### 1. The Preparation of Compound 1c

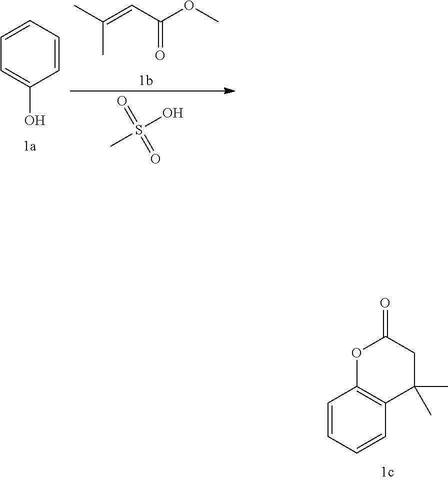

Compound 1a (40 g, 425 mmol, 37.3 mL) was dissolved in CH₃SO₃H (240 mL), and then compound 1b (72.7 g, 637 mmol, 77.8 mL) was added. The mixture was stirred at 70° C. for 96 hours. The reaction mixture was cooled to 0° C. and quenched by the addition of water (500 mL), and then extracted with ethyl acetate (500 mL×2). The organic phase was washed with brine (100 mL×2), dried over anhydrous Na₂SO₄ and filtered. The organic phase was concentrated under reduced pressure and purified by silica gel column chromatography (petroleum ether:ethyl acetate=10:1) to obtain Compound 1c (43.1 g, 57%).

¹H NMR (400 MHz CDCl₃) δ 7.63 (dd, J=1.6 Hz, 3.6 Hz, 1H), 7.24-7.29 (m, 1H), 7.14-7.18 (m, 1H), 7.63 (dd, J=1.2 Hz, 8.0 Hz, 1H), 2.64 (s, 2H), 1.37 (s, 6H).

m/z: ES+[M+H]+ 177.0.

### 2. The Preparation of Compound 1d

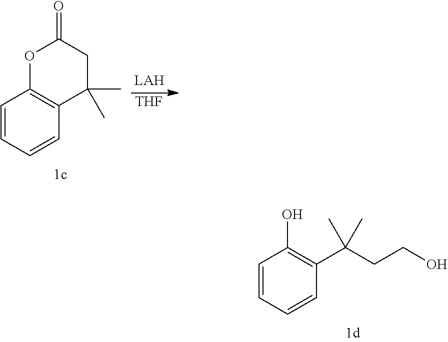

Lithium aluminum hydride (8.61 g, 227 mmol) was suspended in tetrahydrofuran (350 mL), and the atmosphere was replaced with nitrogen three times. Then, a solution of compound 1c (20.0 g, 113 mmol) dissolved in tetrahydrofuran (140 mL) was added dropwise at 0° C. After that, it was stirred at 0° C. for 1.5 hours. Saturated ammonium chloride solution (150 mL) was slowly added to the reaction mixture. The reaction mixture was filtered through celite. The mixture was diluted with ethyl acetate (200 mL×2) and washed twice with brine (300 mL×2). The organic phase was dried over anhydrous sodium sulfate, filtered, and distilled under reduced pressure to obtain Compound 4 (20.4 g, 97%).

¹H NMR (400 MHz CDCl₃) δ 7.22 (dd, J=0.8 Hz, 4.0 Hz, 1H), 7.04-7.10 (m, 1H), 6.82-6.09 (m, 1H), 6.64 (dd, J=0.8 Hz, 4.0 Hz, 1H), 6.17-6.31 (m, 1H), 3.53 (t, J=4.0 Hz, 2H), 2.23 (t, J=4.0 Hz, 2H), 1.42 (6H, s).

### 3. The Preparation of Compound 1f

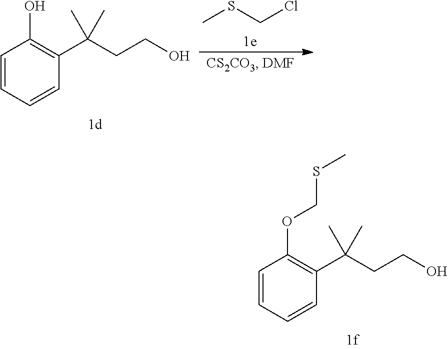

A mixture of Compound 1d (15.0 g, 83.2 mmol) and cesium carbonate (81.3 g, 249 mmol) was subjected to nitrogen replacement three times in DMF (150 mL). Then, Compound 1e (20.0 g, 124 mmol, 17.4 mL) was added, and the reaction solution was stirred at 25° C. for 1 h. The reaction mixture was quenched by adding water (100 mL), and then extracted with ethyl acetate (100 mL×2). The combined organic layers were washed with brine (200 mL×2), dried over anhydrous sodium sulfate, and filtered. The organic phase was concentrated under reduced pressure and purified by silica gel column chromatography (petroleum ether:ethyl acetate=3:1) to afford Compound 1f (10.8 g, 53.9%).

¹H NMR (400 MHz CDCl₃) δ 7.17-7.28 (m, 2H), 6.88-6.98 (m, 2H), 5.2 (s, 2H), 3.45 (t, J=4.0 Hz, 2H), 2.31 (s, 3H), 2.20 (t, J=7.2 Hz, 2H), 1.3 (s, 6H).

### 4. The Preparation of Compound 1g

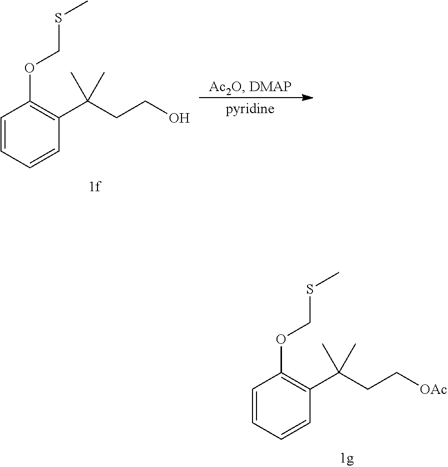 [SMILES: CSCOc1ccccc1C(C)(C)CCO ; CSCOc1ccccc1C(C)(C)CCOC(C)=O]

Compound 1f (10.8 g, 44.9 mmol) was dissolved in pyridine (100 mL), and acetic anhydride (13.7 g, 134 mmol, 12.6 mL) and DMAP (548 mg, 4.49 mmol) were successively added. The reaction mixture was stirred at 25° C. for 2 hours. Ethanol (50 mL) was added to the solution, and the mixture was concentrated under reduced pressure. The resulting residue was dissolved in ethyl acetate (100 mL), and the obtained solution was washed with saturated sodium bicarbonate solution (120 mL) and brine (120 mL). The organic phase was dried over anhydrous sodium sulfate, filtered, and concentrated under reduced pressure to obtain Compound 1g (12.0 g, 94.5%).

¹H NMR (400 MHz CDCl₃) δ 7.16-7.26 (m, 2H), 6.88-6.97 (m, 2H), 5.19 (s, 2H), 3.88 (t, J=4.0 Hz, 2H), 2.31 (s, 3H), 2.25 (t, J=4.0 Hz, 1.6H), 1.89 (s, 3H), 1.43 (s, 6H).

### 5. The Preparation of Compound 1h

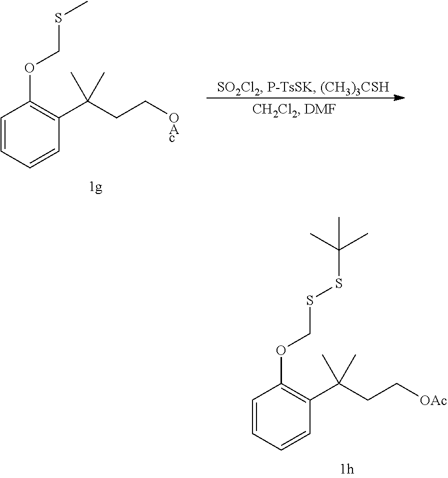 [SMILES: CSCOc1ccccc1C(C)(C)CCOC(C)=O ; CC(=O)OCCC(C)(C)c1ccccc1OCSSC(C)(C)C]

Under a nitrogen atmosphere, SO₂Cl₂ (2.22 g, 16.4 mmol, 1.65 mL) was slowly added to a solution of compound 1g (5.00 g, 17.7 mmol) in dichloromethane (50 mL). The mixture was stirred at 25° C. for 2 hours. The reaction mixture was evaporated to dryness under vacuum. The mixture was dissolved in dichloromethane (20 mL) and DMF (12 mL). p-TsSK (6.01 g, 26.5 mmol) was added to the solution, and the mixture was stirred at 25° C. for 1 hour. Then, tert-butyl mercaptan (3.19 g, 35.4 mmol, 3.99 mL) was added, and the reaction solution was stirred at 25° C. under a nitrogen atmosphere for 18 hours. The reaction solution was diluted with ethyl acetate (120 mL), washed with water (100 mL×3), and the organic phase was dried over anhydrous sodium sulfate, filtered. The organic phase was concentrated under reduced pressure and purified by silica gel column chromatography (petroleum ether:ethyl acetate=10:1) to obtain compound 1h (4.42 g, 69.7%).

¹H NMR (400 MHz CDCl₃) δ 7.14-7.26 (m, 2H), 6.87-6.98 (m, 2H), 5.34 (m, 2H), 3.85 (t, J=4.0 Hz, 2H), 2.25 (t, J=4.0 Hz, 2H), 1.89 (s, 3H), 1.42 (s, 6H), 1.37 (s, 9H).

6. The preparation of Compound 1i

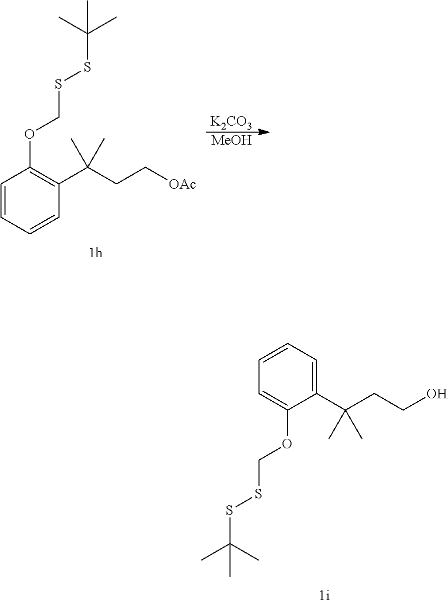 [SMILES: "MeOH" ; CC(=O)OCCC(C)(C)c1ccccc1OCSSC(C)(C)C ; "K2CO3" ; CC(C)(C)SSCOc1ccccc1C(C)(C)CCO]

Compound 1h (4.4 g, 12.3 mmol) and potassium carbonate (3.41 g, 24.6 mmol) were dissolved in methanol (40 mL), and the mixture was stirred at 25° C. for 2 hours. The reaction mixture was diluted with ethyl acetate (50 mL), washed twice with brine (100 mL×2), and the organic layer was dried over anhydrous sodium sulfate, and filtered. The organic phase was concentrated under reduced pressure and purified by silica gel column chromatography (petroleum ether:ethyl acetate=5:1) to obtain compound 1i (1.19 g, 30.8%).

¹H NMR (400 MHz CDCl₃) δ 7.18-7.27 (m, 2H), 6.89-6.99 (m, 2H), 5.35 (s, 2H), 3.43 (t, J=2.0 Hz, 2H), 2.20 (t, J=4.0 Hz, 2H), 1.42 (s, 6H), 1.38 (s, 9H).

### 7. The Preparation of Compound DN0110

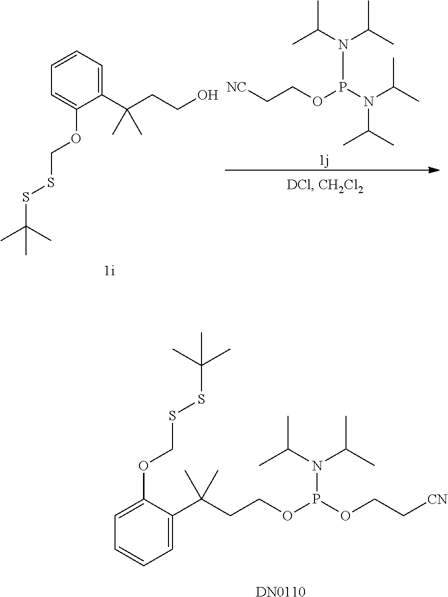

Compound 1i (1.00 g, 3.18 mmol) and DCI (187 mg, 1.59 mmol) were dissolved in a dichloromethane (10 mL) solution, and then compound 1j (1.92 g, 6.36 mmol, 2.02 mL) was added. The reaction mixture was stirred at 25° C. for 1 hour. Triethylamine (0.5 mL) and silica gel basified with triethylamine were added to the reaction solution, which was then concentrated under reduced pressure. The crude product was purified by silica gel column chromatography (petroleum ether (containing 1% triethylamine)/ethyl acetate=98/2) to obtain compound DN0110 (1.03 g, 62.9%).

¹H NMR (400 MHz CDCl₃) δ 7.16-7.27 (m, 2H), 6.89-6.99 (m, 2H), 5.33 (m, 2H), 3.67-3.80 (m, 2H), 3.47-3.59 (m, 2H), 3.30-3.45 (m, 2H), 2.58 (t, J=4.0 Hz, 2H), 2.17-2.25 (m, 2H), 1.41 (s, 6H), 1.37 (s, 9H), 1.15 (t, J=4.0 Hz, 6H), 1.09 (t, J=4.0 Hz, 6H)

³¹P NMR (400 MHz CDCl₃) δ 146.50.

### Example 2 the Preparation of Compound DN0111

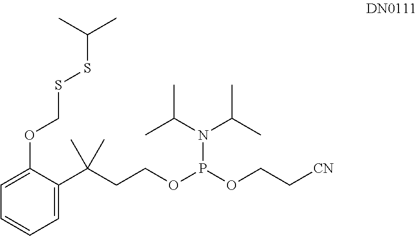 [SMILES: CC(C)SSCOc1ccccc1C(C)(C)CCOP(OCCC#N)N(C(C)C)C(C)C]

The preparation method of Example 2 is referred to Example 1, wherein isopropylmercaptan is used in replacement of tert-butyl mercaptan.

¹H NMR (400 MHz, CDCl₃) δ 7.09-7.17 (m, 2H), 6.82-6.91 (m, 2H), 5.21 (s, 2H), 3.61-3.70 (m, 2H), 3.440-3.57 (m, 4H), 3.18-3.27 (m, 1H), 2.99-3.05 (m, 1H), 2.51 (t, J=4 Hz, 1H), 2.111-2.21 (m, 2H), 1.33-1.35 (m, 6H), 1.24-1.26 (m, 6H), 1.18-1.22 (m, 6H), 1.05-1.08 (m, 6H).

³¹P NMR (400 MHz CDCl₃) δ 146.52.

### Example 3 The Preparation of AE1

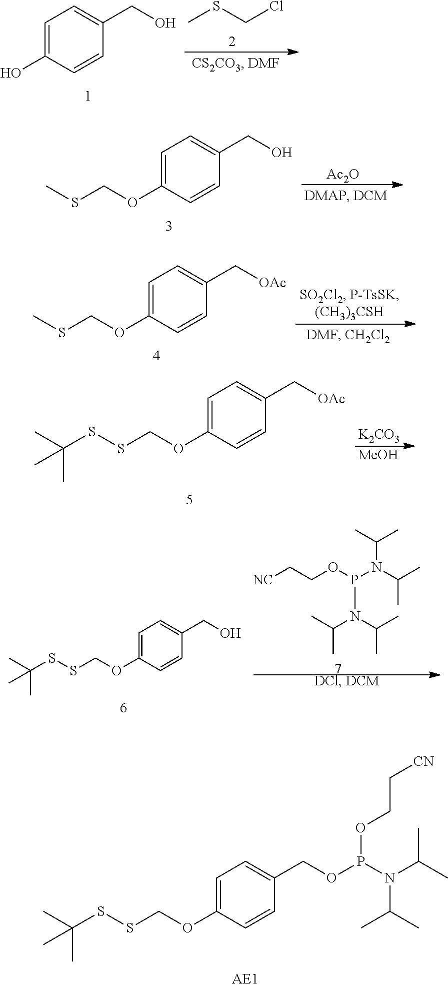

1. The preparation of Compound 3

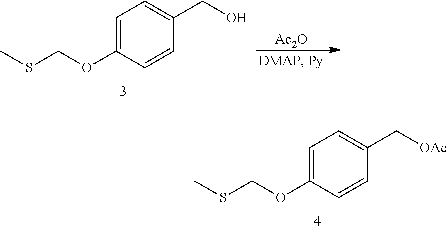

Compound 1 (13.0 g, 104 mmol) and cesium carbonate (102 g, 314 mmol) were dissolved in DMF (130 mL). After purging with nitrogen three times, Compound 2 (15.2 g, 157 mmol, 13.2 mL) was added to the previous solution, and the reaction was carried out at 25° C. for 2 hours. TLC (petroleum ether:ethyl acetate=3:1) indicated the disappearance of the starting materials and the formation of a new compound. The reaction solution was diluted and extracted with ethyl acetate (50 mL×3). The organic phase was washed successively with saturated sodium bicarbonate solution (50 mL) and saturated sodium chloride solution (50 mL), dried over anhydrous sodium sulfate, and evaporated to dryness. The residue was purified by silica gel column chromatography (petroleum ether:ethyl acetate=5:1-3:1) to afford yellow oily Compound 3 (10.0 g, 54.3 mmol, 51.82%).

¹H NMR (400 MHz, CDCl₃) δ 7.32-7.27 (m, 2H), 6.96-6.94 (m, 2H), 5.20 (s, 2H), 4.62 (s, 2H), 2.25 (s, 3H).

### 2. The Preparation of Compound 4

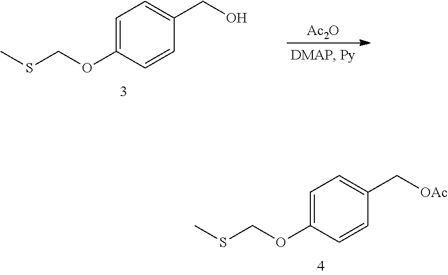

Compound 3 (10 g, 54.27 mmol) was dissolved in pyridine (100 mL), and acetic oxide (6.65 g, 65.13 mmol, 6.12 mL) and 4-dimethylaminopyridine were added. The reaction was carried on under 25° C. for 3 hours. TLC (petroleum ether:ethyl acetate=3:1) showed the disappearance of the starting materials and the generation of a new compound. Ethanol (10 mL) was added to quench the reaction. After the solvent was evaporated by rotary evaporation, extraction was carried out with ethyl acetate (100 mL×3). The organic phase was washed successively with saturated sodium bicarbonate (50 mL) and saturated sodium chloride (50 mL), dried over anhydrous sodium sulfate, evaporated by rotary evaporation, and then purified by silica gel column chromatography (petroleum ether:ethyl acetate=5:1-3:1) to obtain a colorless oily Compound 4 (4 g, 17.32 mmol, 31.92%).

¹H NMR (400 MHz, DMSO) δ 7.31 (d, J=8.78 Hz, 2H), 6.99 (d, J=8.53 Hz, 2H), 5.27 (s, 2H), 5.00 (s, 2H), 2.16 (s, 3H), 2.03 (s, 3H).

### 3. The Preparation of Compound 5

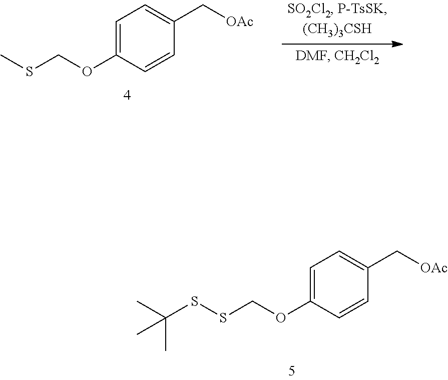 [SMILES: CSCOc1ccc(COC(C)=O)cc1 ; CC(=O)OCc1ccc(OCSSC(C)(C)C)cc1]

Compound 4 (5.0 g, 22.1 mmol) was dissolved in CH₂Cl₂ (50 mL) solution under a nitrogen atmosphere. SO₂Cl₂ (2.98 g, 22.1 mmol, 2.21 mL) dissolved in CH₂Cl₂ (5 mL) was added slowly. The mixture was stirred under 25° C. for 2 hours. The reaction mixture was evaporated under vacuum using a rotary evaporator and then dissolved in CH₂Cl₂ (20 mL) and DMF (12 mL). P-TsSK (7.50 g, 33.1 mmol) was added to the solution and the mixture was stirred under 25° C. for 1 hour. Then tert-butyl mercaptan (3.99 g, 44.2 mmol, 4.98 mL) was added and the reaction was stirred for 18 hours under 25° C. and a nitrogen atmosphere. TLC (petroleum ether:ethyl acetate=10:1) shows the generation of a new compound. The reaction solution was diluted with ethyl acetate (60 mL), washed with water (50 mL×3), and the organic layer was dried over Na₂SO₄, filtered. The organic phase was concentrated under reduced pressure and purified by a normal-phase column (petroleum ether:ethyl acetate=10:1) to obtain a yellow oily Compound 5 (4.90 g, 16.3 mmol, 73.810%). ¹H NMR (400 MHz, CD₃OD) δ 7.32-7.27 (m, 2H), 6.96-6.94 (m, 2H), 5.20 (s, 2H), 4.62 (s, 2H), 2.25 (s, 3H).

### 4. The Preparation of Compound 6

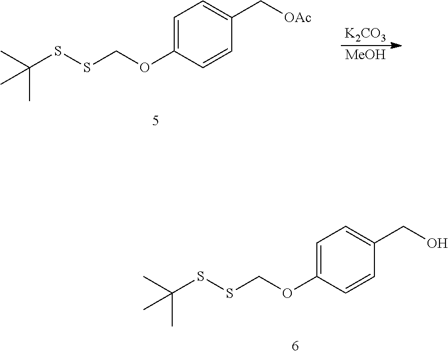

Compound 5 (4.90 g, 16.3 mmol) and K₂CO₃ (4.51 g, 32.6 mmol) were dissolved in MeOH (50 mL) and the mixture was stirred at 25° C. for 2 hours. TLC (petroleum ether:ethyl acetate=3:1) indicated the formation of a new compound. The mixture was concentrated under reduced pressure. Then the reaction mixture was diluted with ethyl acetate (20 mL) and washed twice with brine (50 mL×2). The organic layer was dried over Na₂SO₄. The organic phase was concentrated under reduced pressure and purified by a normal-phase column (petroleum ether:ethyl acetate=3:1) to obtain yellow oily compound 6 (2.1 g, 7.09 mmol, 43.50%).

¹H NMR: (400 MHz, CD₃OD) δ 7.31-7.29 (m, 2H), 6.95-6.91 (m, 2H), 5.33 (s, 2H), 4.50 (s, 2H), 1.32 (s, 9H).

### 5. The Preparation of Compound AE1

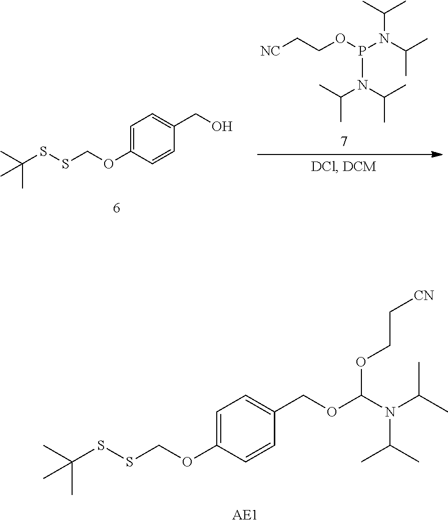 [SMILES: CC(C)(C)SSCOc1ccc(CO)cc1 ; CC(C)N(C(C)C)P(OCCC#N)N(C(C)C)C(C)C ; CC(C)N(C(C)C)C(OCCC#N)OCc1ccc(OCSSC(C)(C)C)cc1]

Compound 6 (800 mg, 3.10 mmol) and DCI (183 mg, 1.55 mmol) were dissolved in a CH₂Cl₂ (8 mL) solution, and then compound 7 (1.87 g, 6.19 mmol) was added. The reaction mixture was stirred at 25° C. for 1 hour. TLC (petroleum ether (containing 1% triethylamine)/ethyl acetate=10/1) showed that the starting materials had completely reacted. Triethylamine (0.5 mL) and silica gel basified with triethylamine were added to the reaction solution, which was then concentrated under reduced pressure. The crude product was separated by normal-phase column chromatography (petroleum ether (containing 1% triethylamine)/ethyl acetate=98/2) to obtain a colorless oily Compound AE1 (560 mg, 1.22 mmol, 39.44%).

¹H NMR (400 MHz, CDCl₃) δ 7.29-7.31 (m, 2H), 6.91-6.93 (m, 2H), 5.29 (s, 2H), 4.59-4.74 (m, 2H), 3.81-3.88 (m, 2H), 3.61-3.70 (m, 2H), 2.63 (t, J=6.8 Hz, 2H), 1.35 (s, 9H), 1.18-1.22 (m, 12H).

### _Example 4 The Preparation of Compound AE2

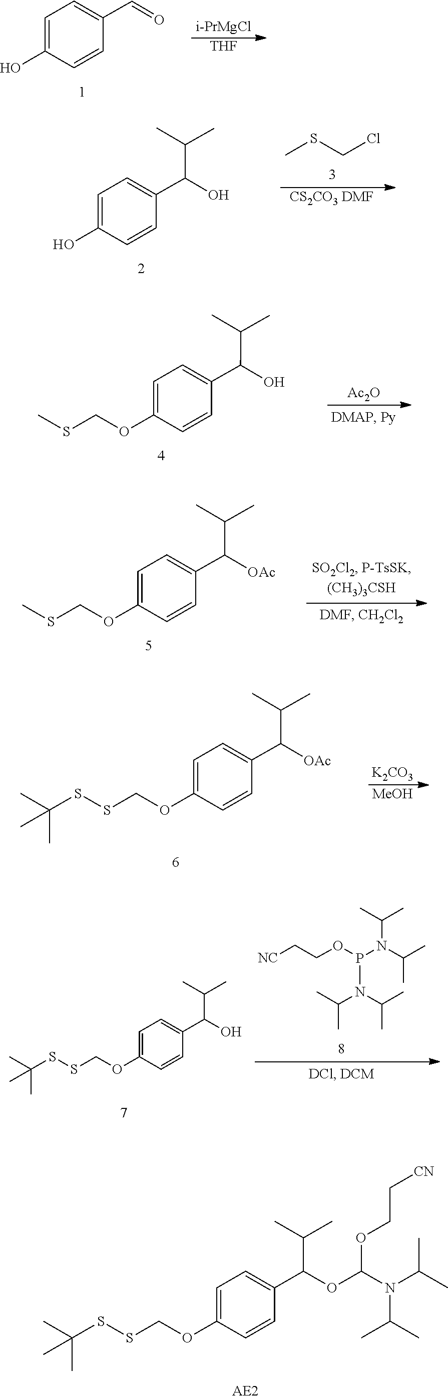

### 1. The Preparation of Compound 2

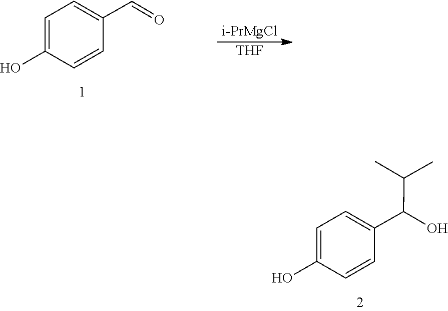

Compound 1 (5.00 g, 40.9 mmol) was dissolved in THF (40 mL). Isopropylmagnesium chloride (i-PrMgCl) (2 M, 81.9 mL) was added at 0° C., and then the reaction was conducted at 0° C. for 3 hours. TLC (petroleum ether:ethyl acetate=3:1) indicated the formation of a new compound. The reaction mixture was cooled to 0° C. and quenched by the addition of ammonium chloride solution (80 mL), and then extracted with ethyl acetate (100 mL×2). The organic phase was washed with brine (100 mL×2), dried over anhydrous Na₂SO₄, and filtered to obtain a brown solid Compound 2 (6.70 g, crude).

¹H NMR (400 MHz, CD₃OD) δ 7.11 (d, J=8.4 Hz, 2H), 6.73 (d, J=8.4 Hz, 2H), 4.15 (d, J=7.2 Hz, 1H), 1.83-1.89 (m, 1H), 0.98 (d, J=7.6 Hz, 3H), 0.72 (d, J=7.6 Hz, 3H).

### 2. The Preparation of Compound 4

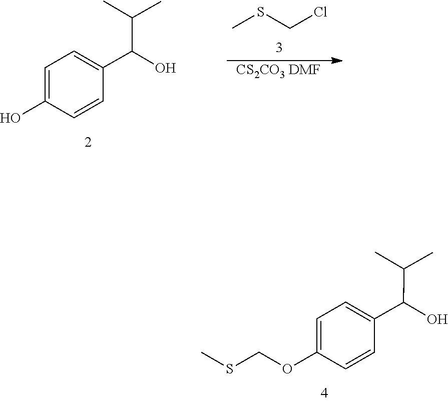

Compound 2 (2 g, 12.0 mmol) and cesium carbonate (11.8 g, 36.1 mmol) were dissolved in DMF (20 mL), and the atmosphere was replaced with nitrogen three times. Then, Compound 3 (1.74 g, 18.1 mmol, 1.51 mL) was added and the mixture was stirred at 25° C. for 1 hour. TLC (petroleum ether:ethyl acetate=3:1) indicated the formation of a new compound. The mixture was diluted with ethyl acetate (40 mL), washed twice with brine (30 mL), and the organic phase was dried over Na₂SO₄, filtered, and evaporated to dryness under reduced pressure to obtain a yellow oily Compound 4 (2.8 g, crude product).

¹H NMR (400 MHz, CD₃OD) δ 7.23 (d, J=8.8 Hz, 2H), 6.93 (d, J=8.8 Hz, 2H), 5.18 (s, 2H), 4.22 (d, J=7.2 Hz, 1H), 2.21 (s, 3H), 1.83-1.93 (m, 1H), 0.99 (d, J=6.4 Hz, 3H), 0.74 (d, J=6.8 Hz, 3H).

### 3. The Preparation of Compound 5

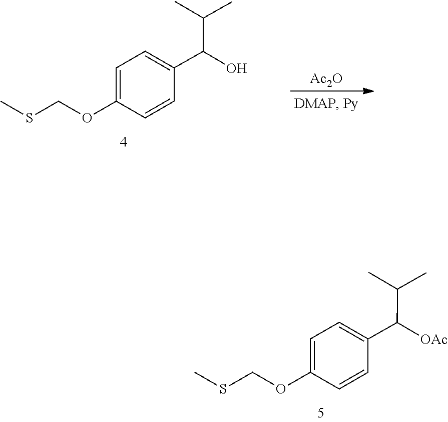 [SMILES: "Ac2O" ; CSCOc1ccc(C(O)C(C)C)cc1 ; CSCOc1ccc(C(OC(C)=O)C(C)C)cc1]

Compound 4 (2.70 g, 11.9 mmol) was dissolved in pyridine (25 mL). Then acetic anhydride (3.65 g, 35.8 mmol, 3.36 mL) and DMAP (146 mg, 1.19 mmol) were successively added. The reaction mixture was stirred at 25° C. for 2 hours. TLC (petroleum ether:ethyl acetate=3:1) showed that a new compound was formed. The mixture was evaporated in vacuo. The residue was dissolved in ethyl acetate (30 mL), and the resulting solution was washed with NaHCO₃ solution (120 mL) and brine (120 mL). The organic layer was dried over Na₂SO₄, filtered under vacuum and concentrated to obtain a yellow oily Compound 5 (2.87 g, 10.69 mmol, 89.65%).

¹H NMR (400 MHz, CD₃OD) δ 7.22 (d, J=8.8 Hz, 2H), 6.94 (d, J=8.8 Hz, 2H), 5.18 (s, 2H), 2.20 (s, 3H), 2.07-2.13 (m, 1H), 2.02 (s, 3H), 1.97 (d, J=8.0 Hz, 3H), 0.78 (d, J=6.8 Hz, 3H).

### 4. The Preparation of Compound 6

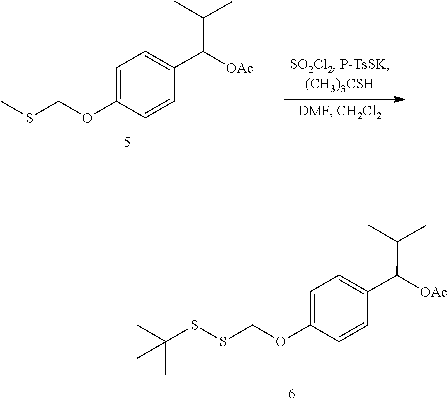 [SMILES: CSCOc1ccc(C(OC(C)=O)C(C)C)cc1 ; CC(=O)OC(c1ccc(OCSSC(C)(C)C)cc1)C(C)C]

Under a nitrogen atmosphere, a solution of SO₂Cl₂ (1.31 g, 9.70 mmol, 970 μL) in CH₂Cl₂ (5 mL) was slowly added to a solution of Compound 5 (2.8 g, 10.4 mmol) in CH₂Cl₂ (30 mL). The mixture was stirred at 25° C. for 2 hours. The reaction mixture was evaporated to dryness under vacuum. The mixture was dissolved in CH₂Cl₂ (10 mL) and DMF (6 mL). P-TsSK (3.54 g, 15.7 mmol) was added to the solution, and the mixture was stirred at 25° C. for 1 hour. Then tert-butyl mercaptan (941 mg, 10.4 mmol, 1.17 mL) was added, and the reaction solution was stirred at 25° C. under a nitrogen atmosphere for 18 hours. TLC (petroleum ether:ethyl acetate=10:1) showed that a new compound was formed. The reaction solution was diluted with ethyl acetate (60 mL) and washed with water (50 mL×3). The organic layer was dried over Na₂SO₄ and filtered. The organic phase was concentrated under reduced pressure and purified by normal-phase column chromatography (petroleum ether:ethyl acetate=10:1) to obtain a yellow oily Compound 6 (2.10 g, 6.13 mmol, 58.7%).

¹H NMR (400 MHz, CD₃OD) δ 7.23-7.26 (m, 2H), 6.91-6.94 (m, 2H), 5.37 (d, J=8.0 Hz, 1H), 5.32 (s, 2H), 2.06-2.12 (m, 1H), 2.04 (s, 3H), 1.32 (s, 9H), 0.97 (d, J=6.4 Hz, 3H), 0.78 (d, J=6.4 Hz, 3H).

### 5. The Preparation of Compound 7

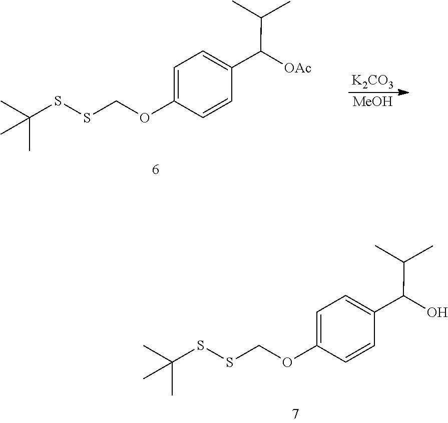

Compound 6 (2.10 g, 6.13 mmol) and K₂CO₃ (1.69 g, 12.3 mmol) were dissolved in MeOH (20 mL), and the mixture was stirred at 25° C. for 2 hours. TLC (petroleum ether:ethyl acetate=10:1) showed that a new compound was formed. The reaction mixture was diluted with ethyl acetate (20 mL), and then it was washed twice with brine (100 mL×2). The organic layer was dried over Na₂SO₄, the organic phase was concentrated under reduced pressure, and was purified by normal phase column chromatography (petroleum ether:ethyl acetate=10:1) to obtain a yellow oily Compound 7 (1.2 g, 3.88 mmol, 63.31%).

¹H NMR (400 MHz, CD₃OD) δ 7.23-7.26 (m, 2H), 6.90-6.93 (m, 2H), 5.33 (s, 2H), 4.22 (d, J=7.2 Hz, 1H), 1.85-1.93 (m, 1H), 1.32 (s, 9H), 0.98 (d, J=6.8 Hz, 3H), 0.74 (d, J=6.8 Hz, 3H).

### 6. The Preparation of Compound AE2

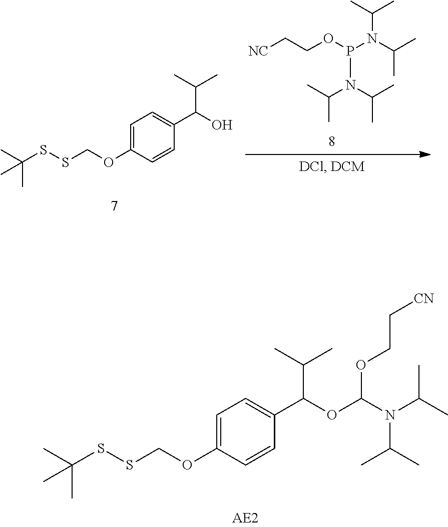 [SMILES: CC(C)C(O)c1ccc(OCSSC(C)(C)C)cc1 ; CC(C)N(C(C)C)P(OCCC#N)N(C(C)C)C(C)C ; CC(C)C(OC(OCCC#N)N(C(C)C)C(C)C)c1ccc(OCSSC(C)(C)C)cc1]

To a solution of Compound 7 (500 mg, 1.66 mmol) and DCI (98.3 mg, 832 μmol) dissolved in CH₂Cl₂ (10 mL), Compound 8 (1.00 g, 3.33 mmol, 1.06 mL) was added. The reaction mixture was stirred at 25° C. for 1 hour. TLC (petroleum ether (1% triethylamine)/ethyl acetate=10/1) indicated that the starting materials were completely reacted. Triethylamine (0.5 mL) and silica gel alkalized with triethylamine were added to the reaction solution, which was then concentrated under reduced pressure. The crude product was separated by normal phase column chromatography (petroleum ether (1% triethylamine)/ethyl acetate=98/2) to obtain a colorless oily Compound AE2 (310 mg, 619 μmol).

¹H NMR (400 MHz, CDCl₃) δ 7.22 (d, J=8.4 Hz, 2H), 6.86-6.91 (m, 2H), 5.28 (d, J=5.6 Hz, 2H), 4.40-4.46 (m, 1H), 3.62-3.89 (m, 2H), 3.48-3.56 (m, 2H), 2.64 (t, J=6.4 Hz, 1H), 2.33 (t, J=6.4 Hz, 1H), 194-1.99 (m, 1H), 1.35 (s, 9H), 1.12-1.23 (m, 9H), 0.91-0.97 (m, 6H), 0.78-0.80 (m, 3H).

### Example 5 the Preparation of Compound AE3

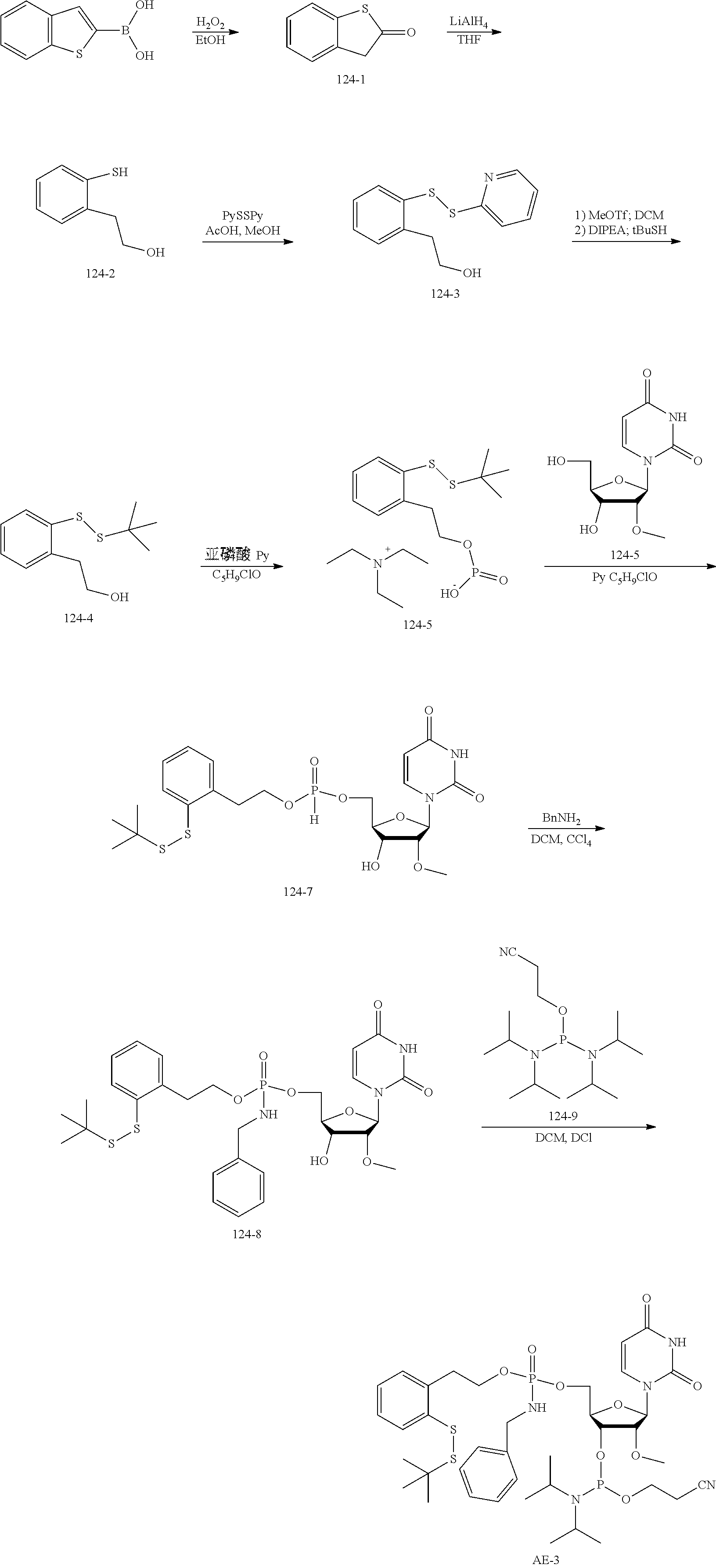

### 1. The Preparation of Compound 124-1

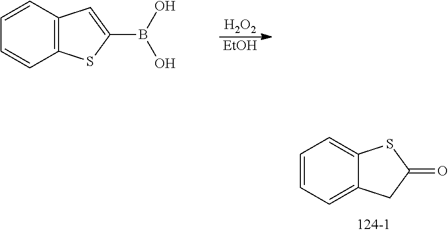 [SMILES: "EtOH" ; OB(O)c1cc2ccccc2s1 ; "H2O2" ; O=C1Cc2ccccc2S1]

Over a period of 30 minutes, 30% hydrogen peroxide (20 mL) was added dropwise to a solution of benzo[b]thiophene-2-boronic acid (10.0 g, 56.2 mmol) in ethanol (50 mL). Then the reaction mixture was stirred for 16 hours. TLC indicated that the reaction was completed. Subsequently, the reaction mixture was carefully washed with water three times and extracted twice with dichloromethane. The organic phase was dried and concentrated to obtain the crude product. Then it was purified by silica gel column chromatography (PE/EA=5:1) to obtain a pale-yellow solid Compound 124-1 (7.8 g, 92.1%).

¹H NMR (400 MHz, CDCl₃) δ 7.50-7.08 (m, 4H), 3.99 (s, 2H).

### 2. The Preparation of Compound 124-2

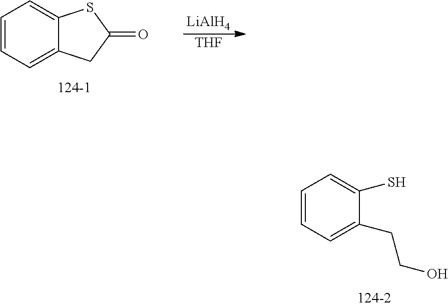 [SMILES: O=C1Cc2ccccc2S1 ; "LiAlH4" ; OCCc1ccccc1S ; "THF"]

A solution of LiAlH₄ (3.9 g, 102.7 mmol) in THF (80 mL) was cooled to 0° C. Then, Compound 124-1 was dissolved in THF (30 mL) and added to the reaction mixture over a period of 1 hour. The reaction was stirred for 16 hours. TLC indicated the completion of the reaction. Subsequently, the reaction mixture was carefully quenched with a saturated Na₂SO₄ solution, and then 1 M HCl solution was added until the reaction mixture became clear. The organic phase was dried and concentrated to obtain the crude product, which was then purified by silica gel column chromatography (PE/EA=2:1) to afford a colorless oily Compound 124-2 (6.0 g, 75.2%).

¹H NMR (400 MHz, CDCl₃) δ 7.23 (dd, J=7.3, 1.8 Hz, 1H), 7.12 (dd, J=7.2, 2.0 Hz, 1H), 7.04 (pd, J=7.3, 1.8 Hz, 2H), 3.81 (t, J=6.6 Hz, 2H), 3.34 (s, 1H), 2.90 (t, J=6.6 Hz, 2H).

### 3. The Preparation of Compound 124-3

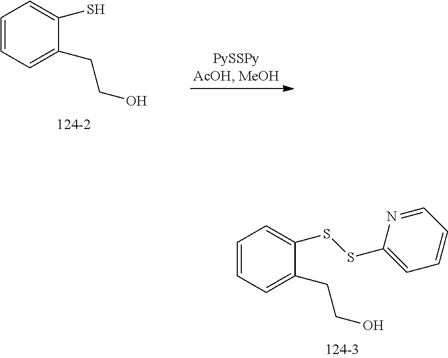 [SMILES: OCCc1ccccc1S ; OCCc1ccccc1SSc1ccccn1]

A solution of Compound 124-2 (6.0 g, 38.9 mmol) in methanol (20 mL) was added to a solution of 2,2′-dipyridyl disulfide (PySSPy, 21.4 g, 97.1 mmol) and acetic acid (1.2 mL) in methanol (100 mL). The reaction was stirred for 16 hours. TLC indicated the completion of the reaction. Subsequently, the reaction was directly concentrated to obtain the crude product, which was purified by silica gel column chromatography to afford a colorless oily Compound 124-3 (5.7 g, 56.0%).

¹H NMR (400 MHz, CDCl₃) δ 8.50 (dt, J=4.9, 1.4 Hz, 1H), 7.64 (ddt, J=8.2, 7.2, 3.2 Hz, 3H), 7.27-7.17 (m, 3H), 7.15-7.11 (m, 1H), 3.97 (t, J=6.6 Hz, 2H), 3.18 (t, J=6.6 Hz, 2H).

### 4. The Preparation of Compound 124-4

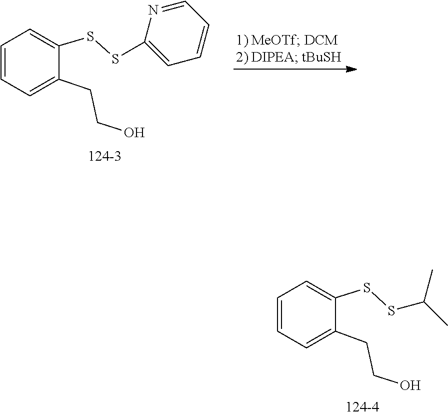 [SMILES: OCCc1ccccc1SSc1ccccn1 ; CC(C)SSc1ccccc1CCO]

Methyl trifluoromethanesulfonate (MeOTf, 7.1 g, 43.4 mmol) was added dropwise to a solution of Compound 124-3 (5.7 g, 21.7 mmol) in dichloromethane (60 mL), and then the reaction mixture was stirred for 15 minutes. Tert-Butyl mercaptan (2.0 g, 21.7 mmol) and DIPEA (7.6 mL, 43.4 mmol) were added to the reaction mixture. Then the reaction mixture was stirred for 30 minutes. TLC showed that the reaction was completed. The reaction mixture was directly concentrated to obtain the crude product, which was purified by silica gel column chromatography (PE/EA=6:1) to afford a colorless oily Compound 124-4 (2.9 g, 55.6%).

¹H NMR (400 MHz, CDCl₃) δ 7.77 (dd, J=7.9, 1.1 Hz, 1H), 7.19-7.05 (m, 3H), 3.84 (t, J=6.8 Hz, 2H), 3.05 (t, J=6.7 Hz, 2H), 1.23 (s, 9H).

### 5. The Preparation of Compound 124-5

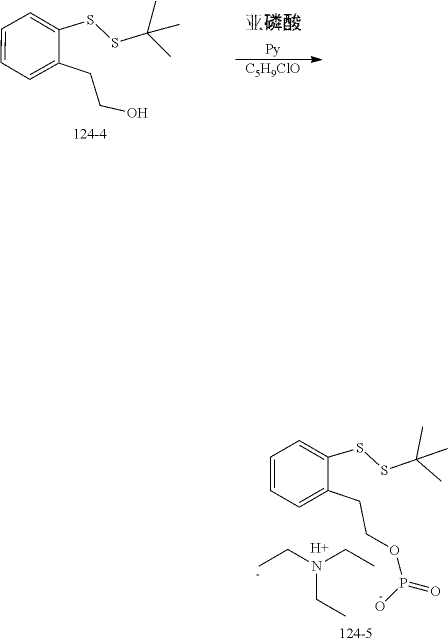

Phosphorous acid (9.8 g, 120.0 mmol) was co-evaporated with anhydrous pyridine three times and then redissolved in pyridine (60 mL). Compound 124-4 (2.9 g, 12.0 mmol) was added to the reaction mixture. Then the reaction mixture was cooled to 0° C. and stirred for 10 minutes. Pivaloyl chloride (7.2 g, 60.0 mmol) was added to the reaction mixture and stirred for 3 hours. The reaction was quenched with triethylammonium bicarbonate buffer (30 mL, 1M) and diluted twice with ethyl acetate. Subsequently, the organic phases were combined and concentrated to obtain the crude product, which was purified by silica gel column chromatography (MeOH:DCM=10:1, 1% TEA) to afford a colorless oily Compound 124-5 (860 mg, 59.7%).

### 6. The Preparation of Compound 124-7

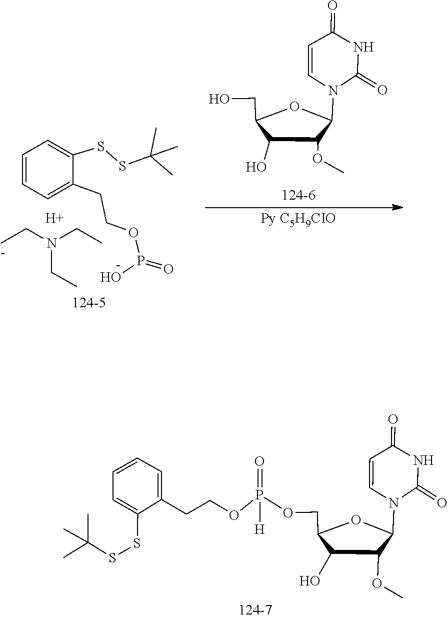

A solution of Compound 124-5 (860 mg, 2.1 mmol) and Compound 124-6 (465.0 mg, 1.8 mmol) was co-evaporated with anhydrous pyridine three times and then redissolved in pyridine (15 mL). The reaction mixture was cooled to −15° C., and pivaloyl chloride (0.5 mL, 3.6 mmol) was added dropwise and stirred at −15° C. for 2 hours. The reaction mixture was diluted with dichloromethane and the reaction was quenched with a saturated NH₄C₁ solution. The organic phases were combined and concentrated to obtain the crude product, which was purified by silica gel column chromatography (MeOH:DCM=5:1, 1% acetic acid) to afford a white solid Compound 124-7 (650 mg, 56.2%).

7. The preparation of Compound 124-8

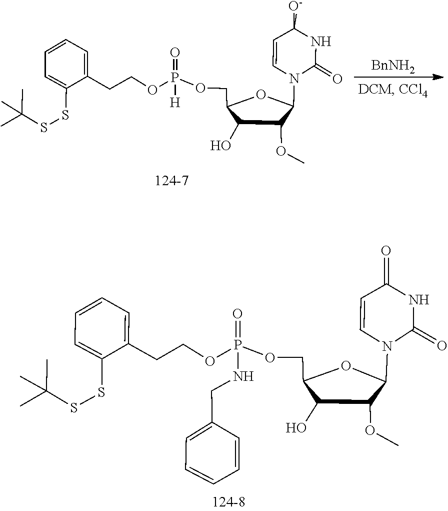

Benzylamine (0.6 mL, 5.5 mmol) was added to a solution of Compound 124-7 (650 mg, 1.2 mmol) in a mixture of dichloromethane and carbon tetrachloride (v/v=1:1, 16 mL), and then the reaction mixture was stirred for 16 hours. TLC indicated that the reaction was completed. Subsequently, the reaction mixture was directly concentrated to obtain the crude product, which was purified by silica gel column chromatography (MeOH:DCM=8:1) to afford a white solid Compound 124-8 (580 mg, 75.0%).

### 8. The Preparation of Compound AE3

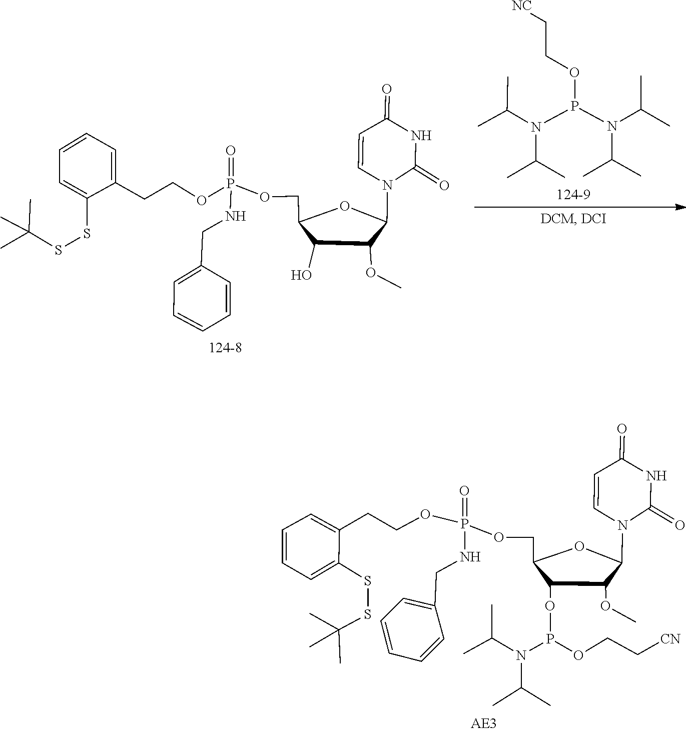

Compound 124-9 (352 mg, 1.2 mmol) and DCI (159.6 mg, 1.4 mmol) were added to a solution of Compound 124-8 (580 mg, 0.9 mmol) in dichloromethane (8 mL), and then the reaction mixture was stirred for 2 hours. TLC showed that the reaction was completed. Subsequently, the reaction mixture was washed with water and extracted with dichloromethane. The organic phases were combined and concentrated to obtain the crude product, which was purified by silica gel column chromatography (PE:EA=6:1, 1% pyridine) to afford a white solid Compound AE3 (640 mg, 85.2%).

¹H NMR (400 MHz, CDCl₃) δ 7.91 (s, 1H), 7.69 (dd, J=8.2, 5.0 Hz, 1H), 7.58 (dd, J=8.2, 3.1 Hz, 1H), 7.35-7.27 (m, 3H), 7.25-7.15 (m, 2H), 6.97-6.91 (m, 1H), 6.87 (d, J=5.0 Hz, 1H), 6.00-5.92 (m, 1H), 5.64 (dd, J=8.2, 6.6 Hz, 1H), 5.56 (dd, J=8.2, 6.7 Hz, 1H), 5.31-5.29 (m, 2H), 4.00 (dtd, J=12.9, 6.8, 6.4, 2.8 Hz, 3H), 3.88-3.74 (m, 5H), 3.69 (tt, J=12.5, 6.3 Hz, 2H), 3.62 (tt, J=12.5, 6.3 Hz, 3H), 3.49 (d, J=1.0 Hz, 1H), 3.45 (d, J=6.0 Hz, 2H), 2.29 (t, J=7.2 Hz, 3H), 1.34 (d, J=1.2 Hz, 9H), 1.19 (ddd, J=15.2, 6.9, 3.6 Hz, 12H). ³¹P NMR (162 MHz, CDCl₃) δ 151.07, 150.78, 150.28, 150.23, 8.92, 8.73, 8.42, 8.31.

### Example 6 The Preparation of Compound AE4

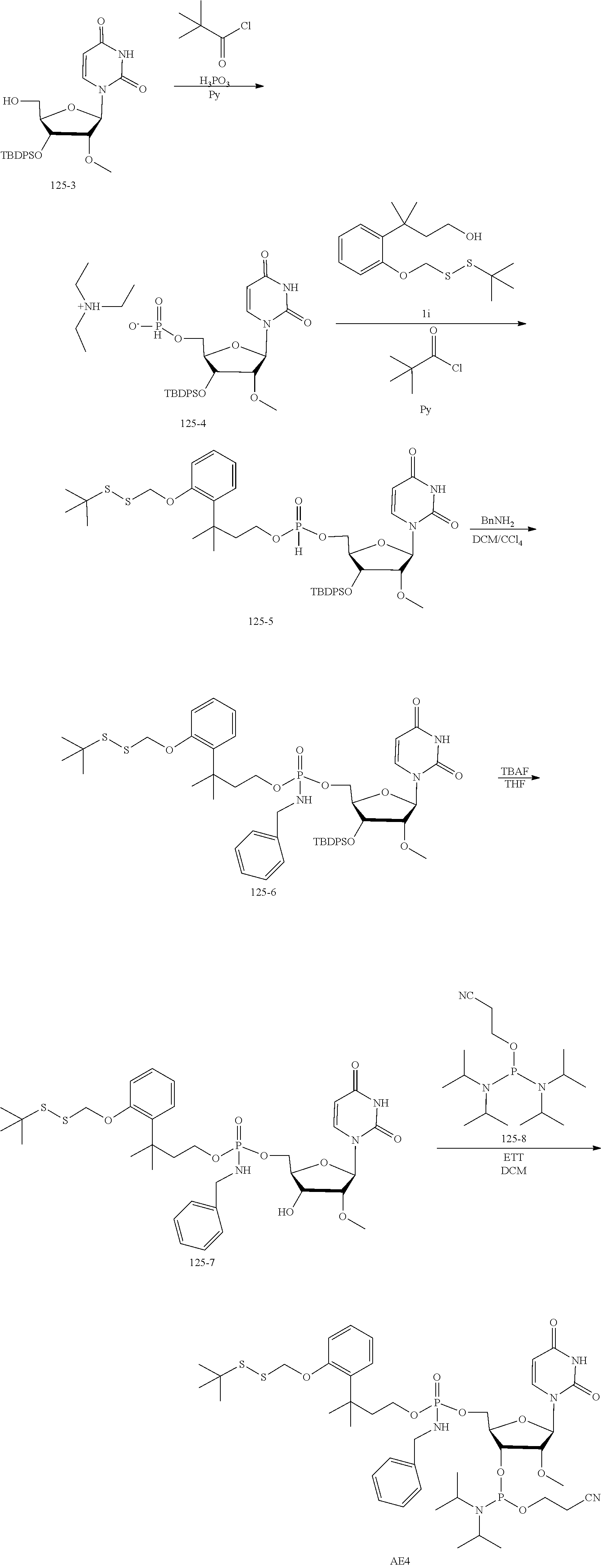

1. The preparation of Compound 125-4

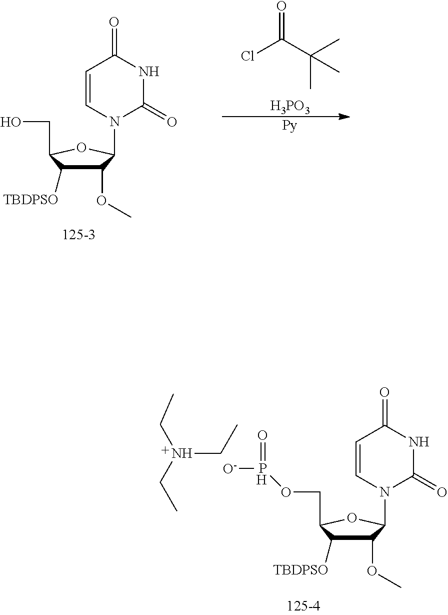

Trimethylacetyl chloride (4.07 mL, 33.73 mmol) was added to a solution of phosphonic acid (5.53 g, 67.45 mmol) and Compound 125-3 (6.70 g, 13.49 mmol) in anhydrous pyridine (Py, 50 mL), and the mixture was stirred. Subsequently, thin-layer chromatography (TLC) was carried out on the reaction mixture. After 3 hours, the reaction was quenched by carefully adding 2 M triethylamine bicarbonate buffer solution (TEAB, 20 mL). After the evolution of carbon dioxide ceased, the reaction mixture was evaporated to dryness, and the residue was partitioned between dichloromethane and 0.5 M TEAB. The organic layer was evaporated, and the residue was purified on a silica gel column to obtain Compound 125-4 (8.40 g, 94%).

### 2. The Preparation of Compound 125-5

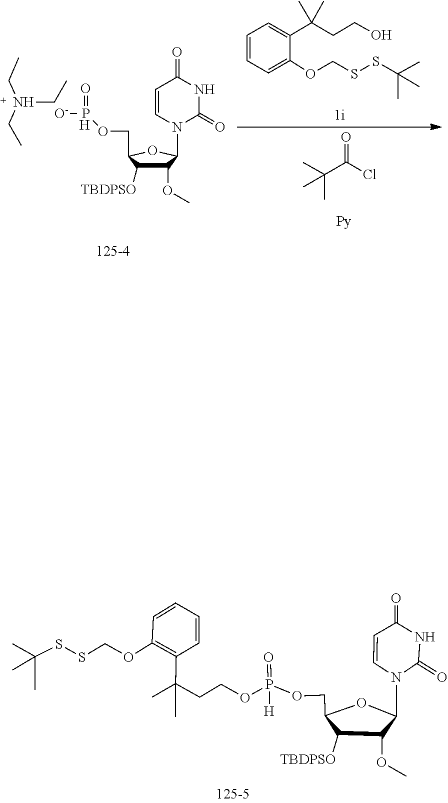

Trimethylacetyl chloride (1.4 g, 11.3 mmol) was added to a stirred solution of Compound 125-4 (2.5 g, 3.8 mmol) and Compound 1i (1.2 g, 3.8 mmol) in anhydrous pyridine (10 mL). After thin-layer chromatography (TLC) analysis showed that the reaction was complete, 2 M TEAB (1 mL) was added. The reaction mixture was concentrated, and the residue was partitioned between dichloromethane and 0.5 M TEAB. The organic layer was collected, dried over Na₂SO₄ and evaporated. Subsequently, purification on a silica gel column was carried out to obtain a diastereomeric mixture of Compound 125-5 (crude, 1.7 g, 53%).

3. The preparation of Compound 125-6

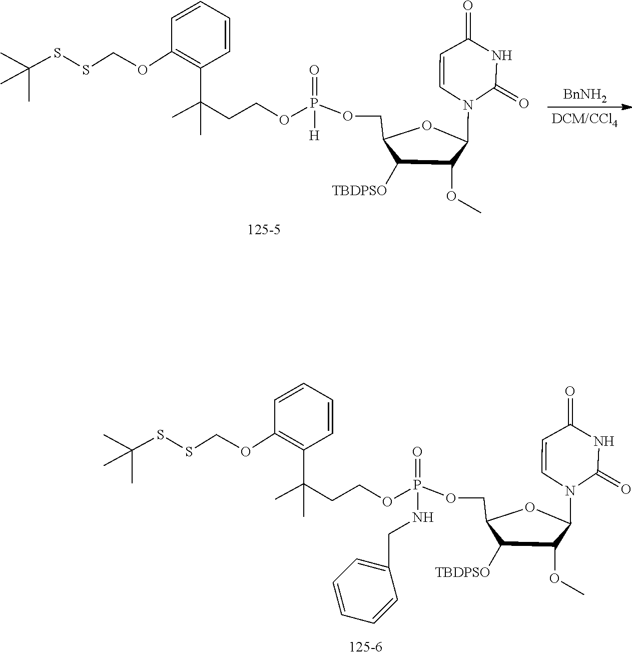

Benzylamine (0.3 g, 3 mmol) was added dropwise to a solution of Compound 125-5 (1.7 g, 2.0 mmol) in a mixture of dichloromethane and carbon tetrachloride (v/v=1:1, 4 ml), and the resulting mixture was stirred for 3 hours. The volatiles were removed under vacuum to obtain a residue, which was then purified by flash silica gel column chromatography to afford a white solid Compound 125-6 (1.5 g, 79%).

### 4. The Preparation of Compound 125-7

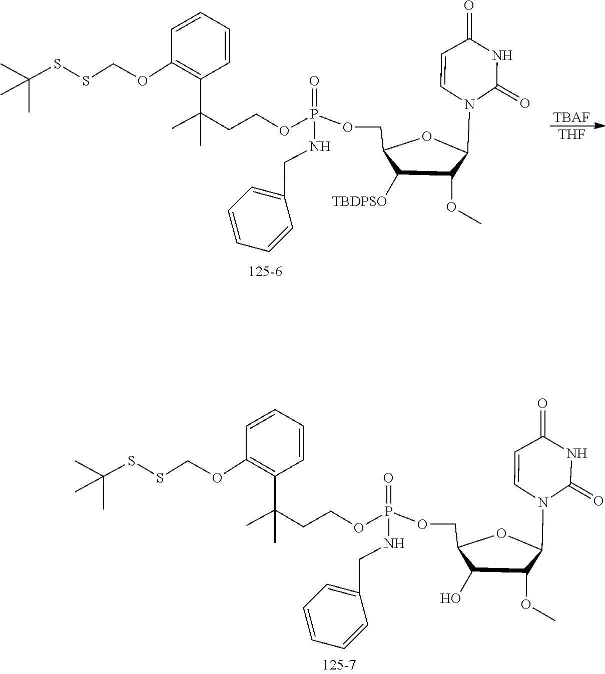

Compound 125-6 was dissolved in a 1 M solution of tetra-n-butylammonium fluoride (TBAF) in tetrahydrofuran (THF, 0.5 mL) and stirred overnight. Subsequently, the reaction mixture was partitioned between water and diethyl ether. The organic layer was collected, dried over Na₂SO₄ and evaporated. Then, purification on a silica gel column was carried out to obtain a diastereomeric mixture of Compound 125-7 (crude, 1.1 g, 97%).

### 5. The Preparation of Compound AE4

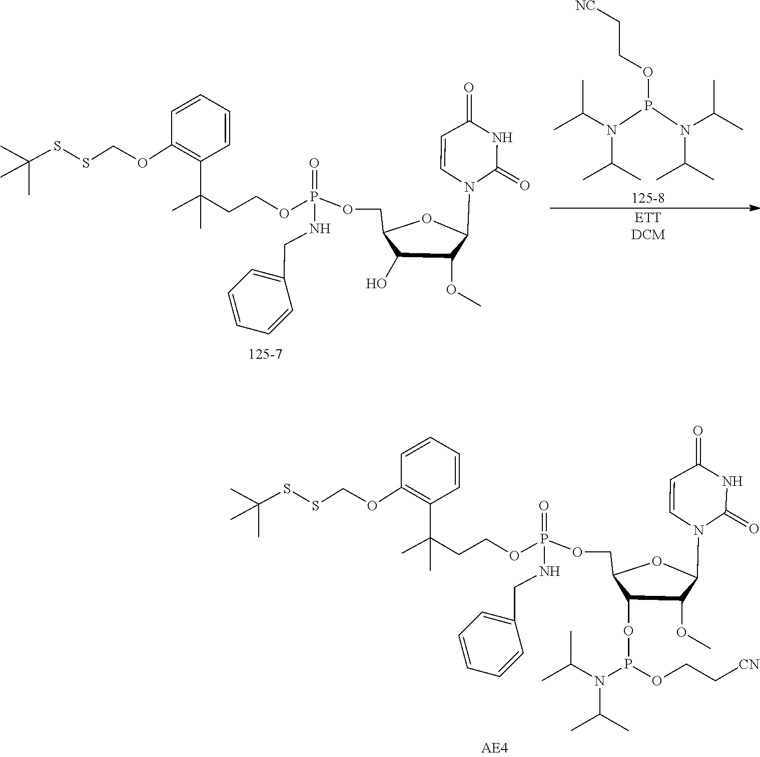

Compound 125-8 (0.37 g, 1.24 mmol) was added to a solution of compound 125-7 (0.60 g, 0.83 mmol) and 5-(ethylthio)-1H-tetrazole (ETT, 0.08 g, 0.66 mmol) in anhydrous dichloromethane (5 mL). The reaction mixture was stirred at room temperature for 2 hours. The reaction mixture was filtered, concentrated and loaded onto a silica gel column to obtain a white foamy Compound AE4 (0.50 g, 65%).

¹H NMR (400 MHz, CDCl₃) δ 7.91 (s, 1H), 7.69 (dd, J=8.2, 5.0 Hz, 1H), 7.58 (dd, J=8.2, 3.1 Hz, 1H), 7.35-7.27 (m, 3H), 7.25-7.15 (m, 4H), 6.97-6.91 (m, 1H), 6.87 (d, J=5.0 Hz, 1H), 6.00-5.92 (m, 1H), 5.64 (dd, J=8.2, 6.6 Hz, 1H), 5.56 (dd, J=8.2, 6.7 Hz, 1H), 5.31-5.29 (m, 2H), 4.41-4.25 (m, 3H), 4.24-4.13 (m, 3H), 4.00 (dtd, J=12.9, 6.8, 6.4, 2.8 Hz, 3H), 3.88-3.74 (m, 5H), 3.62 (tt, J=12.5, 6.3 Hz, 3H), 3.49 (d, J=1.0 Hz, 1H), 3.45 (d, J=6.0 Hz, 2H), 2.98-2.72 (m, 3H), 2.66-2.54 (m, 3H), 2.29 (t, J=7.2 Hz, 3H), 1.40 (s, 6H), 1.34 (d, J=1.2 Hz, 9H), 1.19 (ddd, J=15.2, 6.9, 3.6 Hz, 12H). ³¹P NMR (162 MHz, CDCl₃) δ 151.07, 150.78, 150.28, 150.23, 8.92, 8.73, 8.42, 8.31.

### Example 7 Preparation of Compound AE5

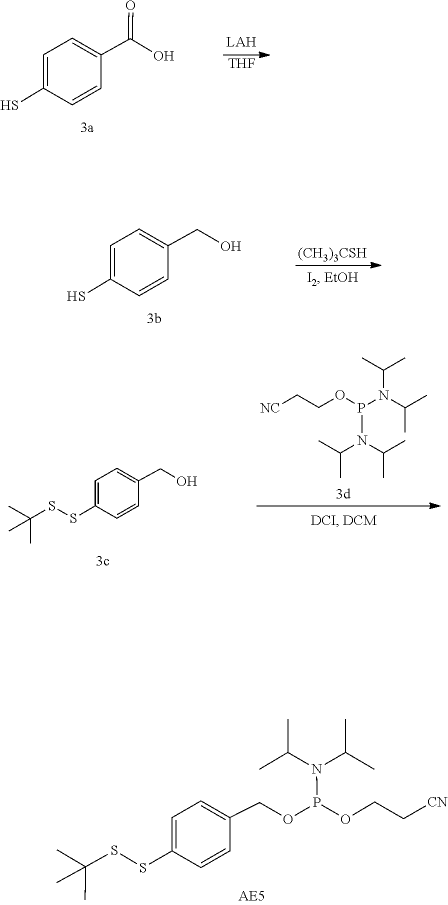

### 1. Preparation of Compound 3b

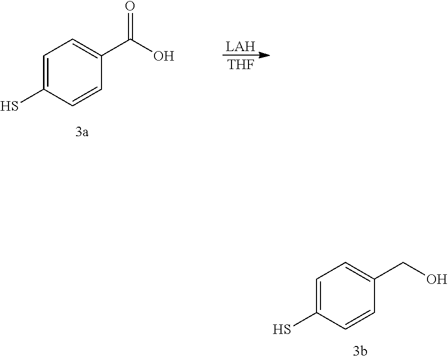

LiAlH₄ (57.08 mg, 142.69 mmol) was added into THF (150 mL) and the reaction was cooled to 0° C. Then Compound 3a (10.00 g, 64.86 mmol) was added. The reaction was conducted under 20° C. for 14 hours and under 25° C. for 24 hours. The reaction was cooled to 0° C. and quenched by 2N hydrochloric acid solution. The aqueous phase was extracted twice with 100 mL ethyl acetate and the organic phase was filtered, and then rotary evaporation to dryness was conducted to obtain compound 3b (8.90 g, 63.48 mmol, 97.9%).

¹H NMR (400 MHz, CD₃OD) δ 7.18-7.28 (m, 4H), 4.54 (s, 2H).

### 2. Preparation of Compound 3c

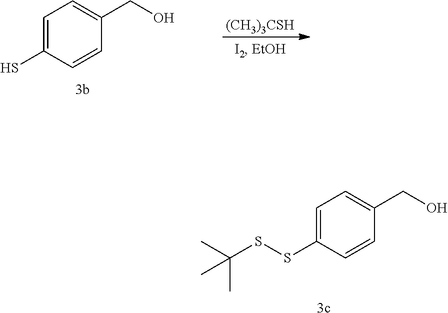

Compound 3b (5.00 g, 35.7 mmol) was dissolved in EtOH (100 mL). tert-Butyl mercaptan (36.95 mL, 340.1 mmol) was added. A solution of iodine (6.79 g, 26.7 mmol) in EtOH (100 mL) was added dropwise at 0° C., and then the reaction was carried out at 20° C. for 2 hours. After the reaction was completed, the pH was adjusted to 7 with saturated sodium bicarbonate. Ethanol was partially removed by rotary evaporation. The aqueous phase was extracted with ethyl acetate (300 mL×3). The organic phase was filtered and evaporated to dryness to obtain a crude product. The crude product was purified by column chromatography (petroleum ether/ethyl acetate) to afford Compound 3c (3.20 g, 14.0 mmol, 39.3%).

¹H NMR (400 MHz, CD₃OD) δ 7.50-7.59 (m, 2H), 7.27-7.35 (m, 2H), 4.57 (s, 2H), 1.28 (s, 9H).

### 3. The Preparation of Compound AE5

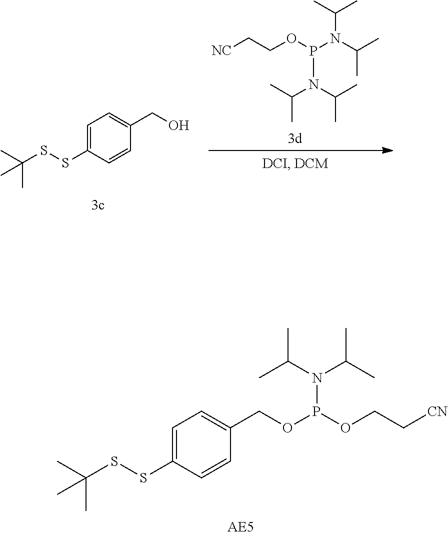 [SMILES: CC(C)(C)SSc1ccc(CO)cc1 ; CC(C)N(C(C)C)P(OCCC#N)N(C(C)C)C(C)C ; CC(C)N(C(C)C)P(OCCC#N)OCc1ccc(SSC(C)(C)C)cc1]

Compound 3c (1.00 g, 4.38 mmol) was dissolved in DCM (10.0 mL). The reaction solution was cooled to 0° C., and DCI (0.57 g, 4.8 mmol) and Compound 3d (2.64 g, 8.76 mmol) were added. The reaction was carried at 0° C. for 1 hour. TLC (petroleum ether:ethyl acetate=3:1) showed the disappearance of the starting materials. 0.3 mL triethylamine and basic silica gel was added to the reaction solution which was then evaporated to dryness. The crude product was purified by column chromatography (petroleum ether:ethyl acetate) to obtain compound AE5 (1.10 g, 2.57 mmol, 58.61%).

¹H NMR (400 MHz, CDCl₃) δ 7.51-7.56 (m, 2H), 7.25-7.35 (m, 2H), 4.55-4.80 (m, 2H), 3.76-3.93 (m, 2H), 3.58-3.71 (m, 2H), 2.60-2.66 (m, 2H), 1.30 (s, 9H), 1.16-1.23 (m, 12H).

### Example 8: Synthesis of siRNA

The siRNA of the present invention was prepared using the solid-phase phosphoramidite method well known in the art. The specific methods can be referred to, for example, PCT Publication Nos. WO2016081444 and WO2019105419, and are briefly described below.

### 1 Synthesis of Sense Strand (SS)

Using the solid-phase phosphoramidite method, a blank CPG solid support or CPG solid support connected with L96 was used as the starting cycle, and nucleoside monomers were linked one by one according to the arrangement of sense strand nucleotides from the 3′ to 5′ direction. Each linkage of a nucleoside monomer involved four steps of deprotection, coupling, capping, and oxidation or thiolation to synthesize 5 μmol oligonucleotide. The synthesis conditions were as follows:

Nucleoside monomers were provided in a 0.05 mol/L acetonitrile solution. The conditions for each step were identical: 25° C.; deprotection for 3 times using a 3% trichloroacetic acid-dichloromethane solution; coupling twice using a 0.25 mol/L 5-(ethylthio)-1H-tetrazole (ETT)-acetonitrile solution as an activator; capping twice using 10% acetic anhydride-acetonitrile and pyridine/N-methylimidazole/acetonitrile (10:14:76, v/v/v); oxidation twice using 0.05 mol/L of iodine in tetrahydrofuran/pyridine/water (70:20:10, v/v/v); thiolation twice using 0.2 mol/L phenylacetyl disulfide (PADS) in acetonitrile/3-methylpyridine (1:1, v/v).

### 2 Synthesis of Antisense Strand (AS)

Using the solid-phase phosphoramidite method, a blank CPG solid support was used as the starting cycle, and nucleoside monomers (including the monomer compound of the present invention) were linked one by one according to the arrangement of antisense strand nucleotides from the 3′ to 5′ direction. Each linkage of a nucleoside monomer involved four steps of deprotection, coupling, capping, and oxidation or thiolation. The conditions for the synthesis of a 5 μmol oligonucleotide for the antisense strand were identical to those for the sense strand.

### 3 Purification and Annealing of Oligonucleotides

### 3.1 Ammonolysis

The synthesized solid support (sense or antisense strand) was transferred to a 5 mL centrifuge tube, followed by the addition of 3% diethylamine/ammonia (v/v) or a mixed solution of ammonia and methylamine (50%-50%, v/v). The mixture was allowed to react in a thermostatic water bath at 35° C. (or 55° C.) for 16 h (or 8 h), and then filtered. The solid support was washed three times with ethanol/water, 1 mL each time. The filtrate was concentrated by centrifugation, and the crude product was purified.

### 3.2 Purification

The methods for purification and desalting are well known to those skilled in the art. For example, a strong anionic packing column can be used; a sodium chloride-sodium hydroxide system can be used for elution and purification. The product can be collected in tubes and desalted using a gel packing purification column. The elution system can be pure water.

### 3.3 Annealing

The sense strand (SS) was mixed with the antisense strand (AS) at a molar ratio (SS/AS=1/1.05) according to the instructions. The mixture was heated in a water bath to 70-95° C. for 3-5 min, and then allowed to cool naturally to room temperature. The system was freeze-dried to obtain the product.

The siRNA sequences as used herein are as follows

| | Sequence (5′->3′) |
|---|---|
| Duplex | The first strand is sense strand and the |
| No. | second strand is antisense strand |
| DR005961 | CfsCmsUfGmGfAmCfAmUfUfCfAmGfAmAfCmAfAmGfAmAf-L96 |
| | (SEQ ID NO: 1) |
| | UmsUfsCmUfUmGfUmUfCmUfGmAmAmUfGmUfCmCfAmGfGmsGfsUm |
| | (SEQ ID NO: 2) |
| DR007069 | CfsCmsUfGmGfAmCfAmUfUfCfAmGfAmAfCmAfAmGfAmAf-L96 |
| | (SEQ ID NO: 1) |
| | RPD1-UmsUfsCmUfUmGfUmUfCmUfGmAmAmUfGmUfCmCfAmGfGmsGfsUm |
| | (SEQ ID NO: 3) |
| DR007071 | CfsCmsUfGmGfAmCfAmUfUfCfAmGfAmAfCmAfAmGfAmAf-L96 |
| | (SEQ ID NO: 1) |
| | RPD2-UmsUfsCmUfUmGfUmUfCmUfGmAmAmUfGmUfCmCfAmGfGmsGfsUm |
| | (SEQ ID NO: 4) |
| DR007073 | CfsCmsUfGmGfAmCfAmUfUfCfAmGfAmAfCmAfAmGfAmAf-L96 |
| | (SEQ ID NO: 1) |
| | RPD3-UmsUfsCmUfUmGfUmUfCmUfGmAmAmUfGmUfCmCfAmGfGmsGfsUm |
| | (SEQ ID NO: 5) |
| DR007074 | CfsCmsUfGmGfAmCfAmUfUfCfAmGfAmAfCmAfAmGfAmAf-L96 |
| | (SEQ ID NO: 1) |
| | RPD4-UmsUfsCmUfUmGfUmUfCmUfGmAmAmUfGmUfCmCfAmGfGmsGfsUm |
| | (SEQ ID NO: 6) |
| DR005969 | CmsAmsCmAmGmAmAfAmCfUfCfAmAmUmAmAmAmGmUmGmAm-L96 |
| | (SEQ ID NO: 7) |
| | UmsCfsAmCmUmUfUmAfUfUmGmAmGmUfUmUfCmUmGmUmGmsCmsCm |
| | (SEQ ID NO: 8) |
| DR007078 | CmsAmsCmAmGmAmAfAmCfUfCfAmAmUmAmAmAmGmUmGmAm-L96 |
| | (SEQ ID NO: 7) |
| | RPD1- |
| | UmsCfsAmCmUmUfUmAfUfUmGmAmGmUfUmUfCmUmGmUmGmsCmsCm |
| | (SEQ ID NO: 9) |
| DR007080 | CmsAmsCmAmGmAmAfAmCfUfCfAmAmUmAmAmAmGmUmGmAm-L96 |
| | (SEQ ID NO: 7) |
| | RPD2- |
| | UmsCfsAmCmUmUfUmAfUfUmGmAmGmUfUmUfCmUmGmUmGmsCmsCm |
| | (SEQ ID NO: 10) |
| DR007082 | CmsAmsCmAmGmAmAfAmCfUfCfAmAmUmAmAmAmGmUmGmAm-L96 |
| | (SEQ ID NO: 7) |
| | RPD3- |
| | UmsCfsAmCmUmUfUmAfUfUmGmAmGmUfUmUfCmUmGmUmGmsCmsCm |
| | (SEQ ID NO: 11) |
| DR007083 | CmsAmsCmAmGmAmAfAmCfUfCfAmAmUmAmAmAmGmUmGmAm-L96 |
| | (SEQ ID NO: 7) |
| | RPD4- |
| | UmsCfsAmCmUmUfUmAfUfUmGmAmGmUfUmUfCmUmGmUmGmsCmsCm |
| | (SEQ ID NO: 12) |
| DR007182 | CfsCmsUfGmGfAmCfAmUfUfCfAmGfAmAfCmAfAmGfAmAf-L96 |
| | (SEQ ID NO: 1) |
| | RPD5-UmsUfsCmUfUmGfUmUfCmUfGmAmAmUfGmUfCmCfAmGfGmsGfsUm |
| | (SEQ ID NO: 13) |
| DR007190 | CmsAmsCmAmGmAmAfAmCfUfCfAmAmUmAmAmAmGmUmGmAm-L96 |
| | (SEQ ID NO: 7) |
| | RPD5- |
| | UmsCfsAmCmUmUfUmAfUfUmGmAmGmUfUmUfCmUmGmUmGmsCmsCm |
| | (SEQ ID NO: 14) |

The abbreviations used herein have the meaning as follows:
A, U, G, and C represent a natural adenine ribonucleotide, a natural uracil ribonucleotide, a natural guanine ribonucleotide, and a natural cytosine ribonucleotide, respectively.
m represents that the left nucleotide is a 2′-OCH₃ modified nucleotide. For example, Am, Um, Gm, and Cm represent 2′-OCH₃ modified A, U, G, and C, respectively.
f represents that the left nucleotide is a 2′-F modified nucleotide. For example, Af, Uf, Gf, and Cf represent 2′-F modified A, U, G, and C, respectively.
“s” or “-s” represents that the flanking two nucleotides, the delivery moiety, or other structures are connected via a phosphorothioate group.
L96 represents the following GalNAc-containing delivery moiety well-known in the art, wherein [symbol] represents attachment to the remainder of the oligonucleotide by a phosphate group or a phosphorothioate group, see for example PCT Publication No. WO2009073809 and WO2009082607.

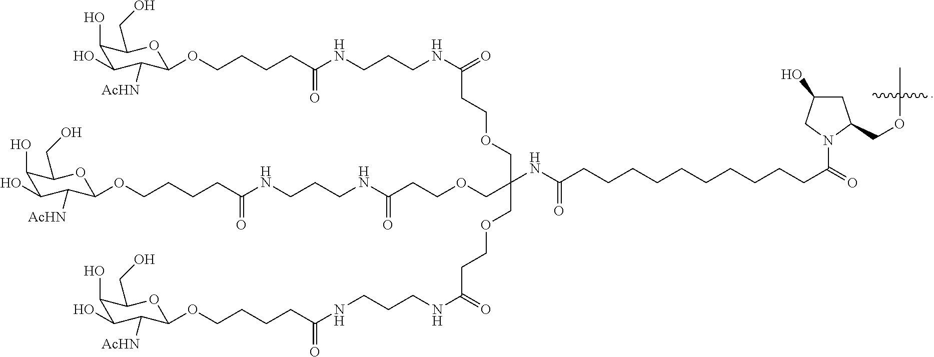 [SMILES: *OC[C@@H]1C[C@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

RPD1-RPD5 represent the following structure, wherein [symbol] represents the bond connecting to the hydroxl of the 5′ carbon of the ribose of the right nucleotide.

[symbol]

### Example 9 Activity Screening in C57BL/6 Widetype Mouse Primary Hepatocyte (PMH)

### 1. Free Uptake or Transfection

the C57BL/6 widetype mouse primary hepatocyte was isolated, counted, and seeded into a 24-well plate at 900 μL/well (8×10⁴ cells/well) or 96-well plate at 100 μL/well (1×10⁴ cells/well) for free uptake and transfection later.

Freeuptake: 10 μL of the diluted compound was added to 90 μL of Opti-MEM and mixed well. Then the mixture was added to the corresponding wells and incubated in an incubator at 37° C. with 5% CO₂ for 24 hours. No siRNA was added to the control group.

Transfection: 10 μL of the diluted compound was added to 40 μL of Opti-MEM and mixed well. 3 μL of RNAiMAX was added to 47 μL of Opti-MEM and mixed well. After incubating for 5 minutes, it was mixed with the diluted compound. The mixture was allowed to stand at room temperature for 10 minutes and then added to the corresponding wells. The samples were incubated in an incubator at 37° C. with 5% CO₂ for 24 hours. No siRNA was added to the control group.

### 2. Fluorescent Quantitative PCR

Cellular RNA was extracted using a high-throughput nucleic acid extractor—magnetic bead method (Hangzhou Allsheng, Auto-pure96, FireGen, FG0412). Fluorescent quantitative PCR was conducted

Fluorescent quantitative PCR (TaqMan™ Fast Advanced Master Mix (ABI, 4444965)) was conducted after reverse transcription (PrimeScript™ II 1st Strand cDNA Synthesis Kit (Takara, 6210B)).

TABLE 1
Primer information

| Primer Name | Sequence (5′-3′) | Fluorescent Group |
|---|---|---|
| mAPOB-PF | GAGGCTTGTCACCCTTCTTT (SEQ ID NO: 15) | |
| mAPOB-PR | GCCTTGTGAGCACCAGTATTA (SEQ ID NO: | |
| | 16) | |
| mAPOB-P | ATCCCTCACTTCCCAGGGTGTAGAAT (SEQ | 5′6-FAM, 3′BHQ1 |
| | ID NO: 17) | |
| mFXII-PF- | AACTACCTGGCTTGGATCCAGAA (SEQ ID | |
| MGB | NO: 18) | |
| mFXII-PR- | CCCAAGGAGCACACAAGGA (SEQ ID NO: | |
| MGB | 19) | |
| mFXII-P-MGB | CATATTGCTTCATAACTAAC (SEQ ID NO: | 5′6-FAM, 3′MGB |
| | 20) | |
| mGAPDH-PF | CGGCAAATTCAACGGCACAG (SEQ ID NO: | |
| | 21) | |
| mGAPDH-PR | CCACGACATACTCAGCACCG (SEQ ID NO: | |
| | 22) | |
| mGAPDH-P | ACCATCTTCCAGGAGCGAGACCCCACT | 5′TET, 3′BHQ2 |
| | (SEQ ID NO: 23) | |

### 3. Data Statistics

The 2^{-ΔΔCt} value was calculated and converted into a percentage to obtain the residual inhibition.

ΔΔCt=[(Ct target gene of experimental group−Ct internal reference of experimental group)−(Ct target gene of control group−Ct internal reference of control group)].
wherein the target gene is mAPOB or mFXII, and the internal reference gene is mGAPDH.
   4. Experimental Data of mAPOB

Using the transfection method, C57BL/6 wildtype mouse primary hepatocytes were selected. After seeding the cells in a 24-well plate, the initial concentration of the compound was set at 40 nM, and 5 concentration points were obtained by 10-fold serial dilution (40 nM, 4 nM, 0.4 nM, 0.04 nM, 0.004 nM). A 5-point IC50 activity screening was performed on the C57BL/6 wildtype mouse primary hepatocytes. The experimental results are shown in Table 2.

TABLE 2
PMH 5-point IC50 activity screening of the mAPOB-targetting
siRNA having the compounds of the present invention

| | Duplex | 40 | 4 | 0.4 | 0.04 | 0.004 | IC50 |
|---|---|---|---|---|---|---|---|
| | No. | nM | nM | nM | nM | nM | (nM) |
| 1 | DR005961 | 3.6% | 6.8% | 29.5% | 54.4% | 87.3% | 0.0617 |
| 2 | DR007069 | 2.9% | 5.7% | 11.6% | 35.9% | 74.9% | 0.0174 |
| 3 | DR007071 | 3.5% | 5.4% | 12.8% | 49.3% | 76.7% | 0.0407 |
| 4 | DR007073 | 3.4% | 25.2% | 69.2% | 75.4% | 77.2% | 1.4223 |
| 5 | DR007074 | 8.9% | 39.1% | 76.1% | 63.9% | 83.0% | 3.4995 |

5. Experimental Data of mFXII

Using the free uptake method, C57BL/6 wildtype mouse primary hepatocytes were selected. After seeding the cells in a 96-well plate, the initial concentration of the compound was set at 100 nM, and 5 concentration points were obtained by 10-fold serial dilution (100 nM, 10 nM, 1 nM, 0.1 nM, 0.01 nM). A 5-point IC50 activity screening was performed on the C57BL/6 wildtype mouse primary hepatocytes. The experimental results are shown in Table 3.

TABLE 3
PMH 5-point IC50 activity screening of the mFXII-targetting
siRNA having the compounds of the present invention

| | Duplex | 100 | 10 | 1 | 0.1 | 0.01 | IC50 |
|---|---|---|---|---|---|---|---|
| | No. | nM | nM | nM | nM | nM | (nM) |
| 1 | DR005969 | 0.48 | 0.63 | 0.94 | 0.97 | 1.05 | 50.119 |
| 2 | DR007078 | 0.02 | 0.03 | 0.11 | 0.56 | 0.87 | 0.1230 |
| 3 | DR007080 | 0.02 | 0.04 | 0.21 | 0.66 | 0.83 | 0.2344 |
| 4 | DR007082 | 0.41 | 0.57 | 0.74 | 0.89 | 0.98 | 25.119 |
| 5 | DR007083 | 0.73 | 0.80 | 0.90 | 0.89 | 0.95 | >100 |

### Example 10 Efficacy Verification of Compounds in C57BL/6 Mouse Model

The C57BL/6 mice (male, weighing 18-21 g, 6-8 weeks old, provided by Sparfloxacin (Suzhou) Biotechnology Co., Ltd.) were randomly grouped, with 9 animals in each group. On the 14th day and the 28th day, the mice (N=3 per group) will be euthanized, and the livers will be harvested. The dosage administered to each animal was calculated according to its body weight, and a single dose was administered by subcutaneous injection. The siRNA conjugate was provided to the CRO company as a solution with a concentration of 10 mg/mL (using 0.9% sodium chloride aqueous solution as the solvent). Specifically, before the experiment, the siRNA conjugate was dissolved with 0.9% sodium chloride aqueous solution and diluted to the required solution concentration and volume. The administration volume of the saline and the siRNA conjugate was 5 mL/kg.

Blood samples were collected for detection before the administration (recorded as day −2), and the mice were grouped according to the LDL level on day 2. The remaining samples were stored for the detection of the target protein. Blood was collected from the orbital venous plexus of the mice on days 7, 14, 21, 28 and 42 after the administration (recorded as day 0), and the mice were starved for 5 hours before each blood collection. The concentration of serum LDL (using the Meikang brand low-density lipoprotein cholesterol detection reagent) was dected by Suzhou Anling by the direct method (using the Neusoft automatic biochemical analyzer, NT-1000) at each time point. The remaining samples were stored for the detection of the target protein by the ELISA method (Mouse ApoB ELISA Kit, Abcam, ab230932). 10 mg of liver tissues (n=3 per group per time point) were collected on days 14, 28 and 42, placed in RNAlater solution, stored at −80° C., and sent with dry ice for the extraction and detection of the mRNA expression in the liver (the detection primers are shown in Table 4 and need to be updated at subsequent time points). The experimental results are shown in Table 5.

TABLE 4
Primer information

| Primer | | Fluorescent |
|---|---|---|
| Name | Sequence (5′-3′) | Group |
| mAPOB-PF | GAGGCTTGTCACCCTTCTTT | |
| | (SEQ ID NO: 15) | |
| mAPOB-PR | GCCTTGTGAGCACCAGTATTA | |
| | (SEQ ID NO: 16) | |
| mAPOB-P | ATCCCTCACTTCCCAGGGTGTAGAAT | 5′6-FAM, |
| | (SEQ ID NO: 17) | 3′BHQ1 |
| mGAPDH-PF | CGGCAAATTCAACGGCACAG | |
| | (SEQ ID NO: 21) | |
| mGAPDH-PR | CCACGACATACTCAGCACCG | |
| | (SEQ ID NO: 22) | |
| mGAPDH-P | ACCATCTTCCAGGAGCGAGACCCCACT | 5′TET, |
| | (SEQ ID NO: 23) | 3′BHQ2 |

TABLE 5
Compound efficacy of siRNA compounds
in C57BL/6 mouse model

| | | Average LDL level (normalized to predose) | | | |
|---|---|---|---|---|---|
| | | Day 2 prior to dosing | | Day 7 | |
| | Compound | Mean | | Mean | |
| | name | pecentage | SD | pecentage | SD |
| 1 | Saline | 100.0% | 30.9% | 94.4% | 20.5% |
| 2 | DR005961 | 100.0% | 21.4% | 55.6% | 13.6% |
| 3 | DR007069 | 100.0% | 18.8% | 37.3% | 10.0% |

### Example 11 Activity Screening in C57BL/6 Widetype Mouse Primary Hepatocyte (PMH)

According to the method in Example 9, more compounds of the present invention were tested.

TABLE 6
PMH 5-point IC50 activity screening of the mAPOB-targetting
siRNA having the compounds of the present invention

| | Duplex | 40 | 4 | 0.4 | 0.04 | 0.004 | IC50 |
|---|---|---|---|---|---|---|---|
| | No. | nM | nM | nM | nM | nM | (nM) |
| 1 | DR005961 | 17.4% | 27.0% | 76.3% | 93.8% | 99.7% | 0.8841 |
| 2 | DR007182 | 6.4% | 6.7% | 19.2% | 91.2% | 99.3% | 0.1446 |

TABLE 7
PMH 5-point IC50 activity screening of the mFXII-targetting
siRNA having the compounds of the present invention

| | Duplex | 100 | 10 | 1 | 0.1 | 0.01 | IC50 |
|---|---|---|---|---|---|---|---|
| | No. | nM | nM | nM | nM | nM | (nM) |
| 1 | DR005969 | 9.7% | 30.4% | 82.4% | 112.7% | 140.4% | 4.0272 |
| 2 | DR007190 | 0.9% | 1.4% | 3.4% | 26.1% | 75.0% | 0.0375 |

1. An oligonucleotide comprising a compound of formula (II), or a pharmaceutically acceptable salt, tautomer or stereoisomer thereof: 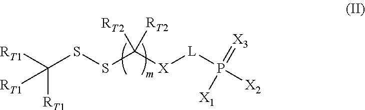
wherein,
X₁ is selected from ORₐ or NR_{b}R_{c};
Rₐ is selected from H, C₁₋₆ alkyl or C₁₋₆ haloalkyl, which is optionally deuterated or fully deuterated;
R_{b} and R_{c} is selected from H, C₁₋₆ alkyl or C₁₋₆ haloalkyl; R_{b} and R_{c} may be optionally substituted with D, C₆₋₁₀ aryl or 5-10-membered heteroaryl, or fully deuterated;
X₂ is the remainder of the oligonucleotide, which connects to P via a hydroxyl or a sulfydryl on the 2′, 3′ or 5′ carbon of the ribose of its first nucleotide counting from the 5′ end;
X₃ is independently selected from O or S;
each R_{T1} is independently selected from H, D, C₁₋₆ alkyl, C₁₋₆ haloalkyl, C₂₋₆ alkenyl, C₂₋₆ alkynyl or GalNAc-containing chain, which is optionally deuterated or fully deuterated;
each R_{T2} is independently selected from H, D, C₁₋₆ alkyl, C₁₋₆ haloalkyl, C₂₋₆ alkenyl or C₂₋₆ alkynyl, which is optionally deuterated or fully deuterated;
m is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₆ alkyl or C₁₋₆ haloalkyl, which is optionally deuterated or fully deuterated;
L is —Ar—(CH₂)₁₋₆—O—, wherein each CH₂ may be optionally substituted with one or two R #; R # is selected from H, D, C₁₋₆ alkyl or C₁₋₆ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2, 3, 4 or 5 R*;
R* is selected from C₁₋₆ alkyl, C₁₋₆ haloalkyl, C₂₋₆ alkenyl or C₂₋₆ alkynyl, which is optionally deuterated or fully deuterated.

2. The oligonucleotide of claim 1, or a pharmaceutically acceptable salt, tautomer or stereoisomer thereof, wherein,
X₁ is selected from ORₐ or NR_{b}R_{c};
Rₐ is selected from H, C₁₋₄ alkyl or C₁₋₄ haloalkyl, which is optionally deuterated or fully deuterated;
R_{b} and R_{c} are independently selected from H, C₁₋₄ alkyl, C₁₋₄ haloalkyl; R_{b} and R_{c} may optionally be substituted with D, C₆₋₁₀ aryl, or fully deuterated;
X₂ is the remainder of the oligonucleotide, which connects to P via a hydroxyl or a sulfydryl on the 5′ carbon of the ribose of its first nucleotide counting from the 5′ end;
X₃ is independently selected from O or S;
each R_{T1} is independently selected from H, D, C₁₋₄ alkyl, C₁₋₄ haloalkyl or GalNAc-containing chain, which is optionally deuterated or fully deuterated;
each R_{T2} is independently selected from H, D, C₁₋₄ alkyl or C₁₋₄ haloalkyl, which is optionally deuterated or fully deuterated;
m is 0, 1, 2 or 3;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is —Ar—(CH₂)—O—, wherein CH₂ may optionally be substituted with 1 or 2 R #; R # is selected from H, D, C₁₋₄ alkyl or C₁₋₄ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₄ alkyl or C₁₋₄ haloalkyl, which is optionally deuterated or fully deuterated.

3. The oligonucleotide of claim 1, or a pharmaceutically acceptable salt, tautomer or stereoisomer thereof, wherein,
X₁ is selected from ORₐ or NR_{b}R_{c};
Rₐ is selected from H, C₁₋₄ alkyl or C₁₋₄ haloalkyl, which is optionally deuterated or fully deuterated;
R_{b} and R_{c} are independently selected from H, C₁₋₄ alkyl, C₁₋₄ haloalkyl; R_{b} and R_{c} may optionally be substituted with D, phenyl, or fully deuterated;
X₂ is the remainder of the oligonucleotide, which connects to P via a hydroxyl or a sulfydryl on the 5′ carbon of the ribose of its first nucleotide counting from the 5′ end;
X₃ is independently selected from O or S; 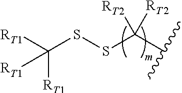
is selected from 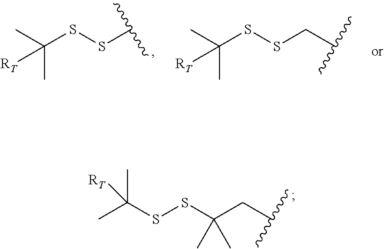
wherein R_{T} is selected from H, D, CH₃ or GalNAc-containing chain, which is optionally deuterated or fully deuterated;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is selected from

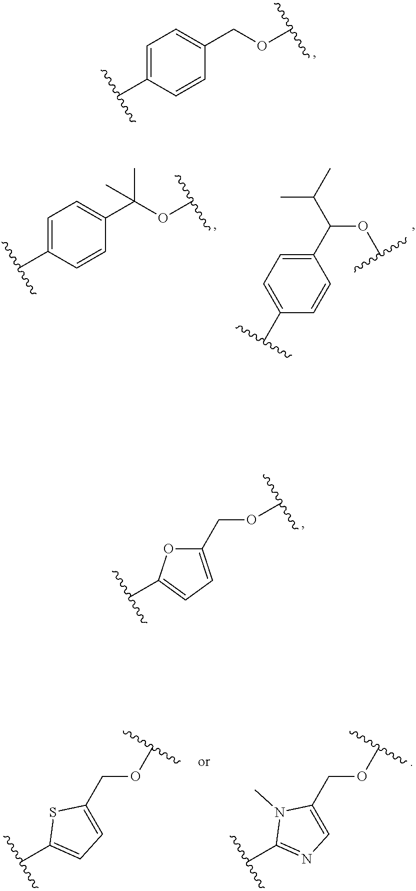

4. The oligonucleotide of claim 1, or a pharmaceutically acceptable salt, tautomer or stereoisomer thereof, wherein,
X₁ is selected from OH or 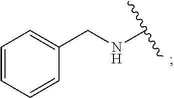 [SMILES: *NCc1ccccc1]
X₂ is the remainder of the oligonucleotide, which connects to P via a hydroxyl or a sulfydryl on the 5′ carbon of the ribose of its first nucleotide counting from the 5′ end;
X₃ is independently selected from O or S; 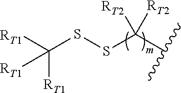
is selected from 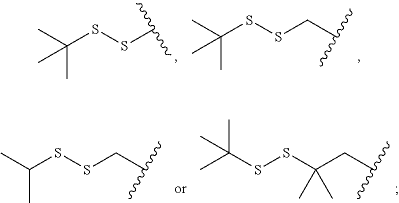
X is selected from bond, —O—, —NHC(O)O—, —OC(O)NH—, —N(CH₃)C(O)O— or —C(O)O—;
L is selected from

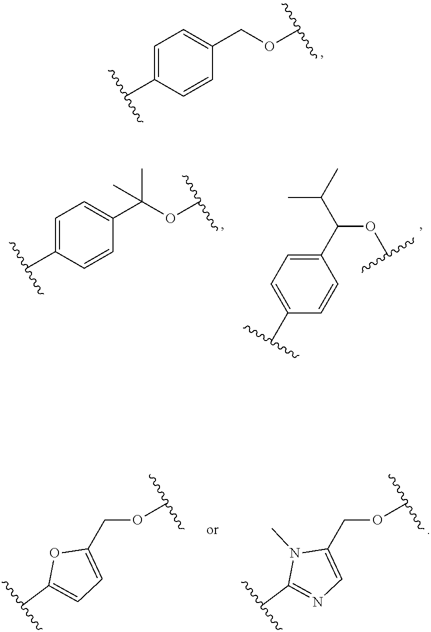

5. The oligonucleotide of claim 1, or a pharmaceutically acceptable salt, tautomer or stereoisomer thereof, the compound of formula (II) is selected from: 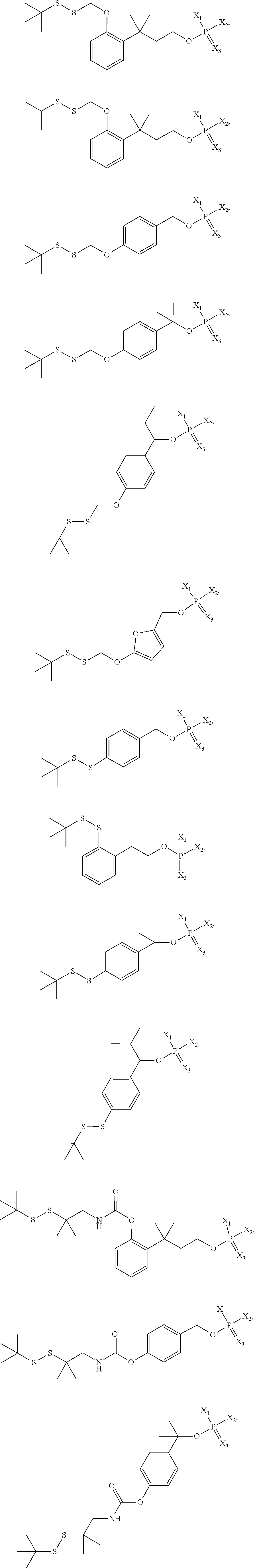 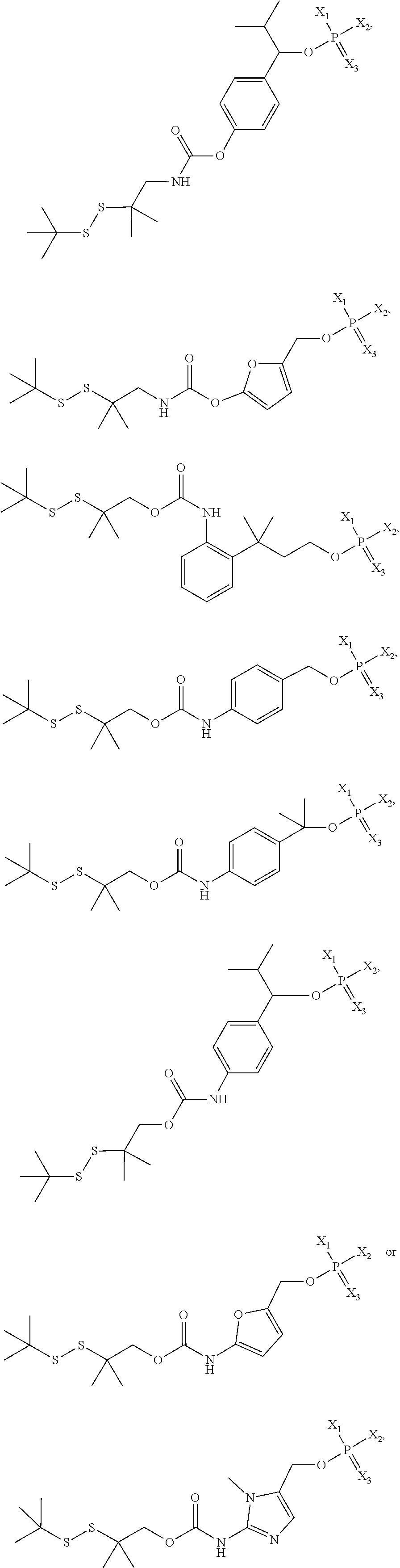
wherein,
X₁ is selected from OH or 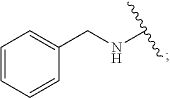 [SMILES: *NCc1ccccc1]
X₂ is the remainder of the oligonucleotide, which connects to P via a hydroxyl or a sulfydryl on the 5′ carbon of the ribose of its first nucleotide counting from the 5′ end;
X₃ is independently selected from O or S;
preferably, the compound of formula (I) is selected from 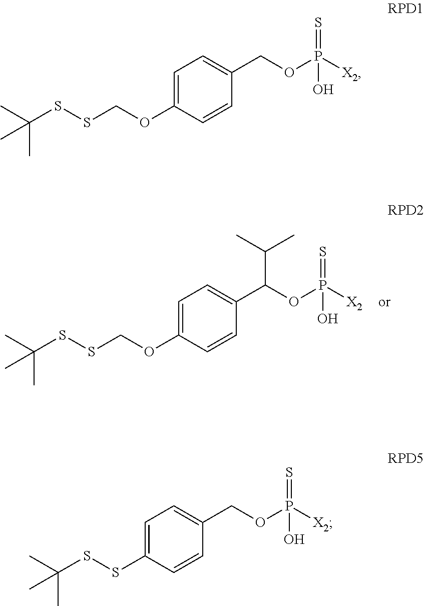
X₂ is the remainder of the oligonucleotide, which connects to P via a hydroxyl or a sulfydryl on the 5′ carbon of the ribose of its first nucleotide counting from the 5′ end.

6. An oligonucleotide, comprising one, two or more compounds of formula (Ia), or a pharmaceutically acceptable salt, tautomer or stereoisomer thereof: 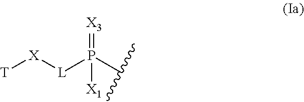
wherein, [symbol]
is selected from ORₐ or NR_{b}R_{c}, or represents a bond connecting to a hydroxyl or a sulfydryl of the 5′ carbon of the ribose of an adjacent nucleotide;
X₁ is a bond connecting to a hydroxyl or a sulfydryl of the 2′ or 3′ carbon of the ribose of the other adjacent nucleotide;
Rₐ is selected from H, C₁₋₆ alkyl, C₁₋₆ haloalkyl, C₂₋₆ alkenyl or C₂₋₆ alkynyl, which is optionally deuterated or fully deuterated;
R_{b} and R_{c} is independently selected from H, C₁₋₆ alkyl or C₁₋₆ haloalkyl; R_{b} and R_{c} may optionally be substituted with D, C₆₋₁₀ aryl or 5-10-membered heteroaryl, or fully deuterated;
X₃ is independently selected from O or S;
T is 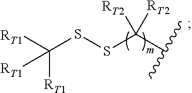
each R_{T1} is independently selected from H, D, halogen, CN, C₁₋₆ alkyl, C₁₋₆ haloalkyl, C₁₋₆ haloalkyl, C₂₋₆ alkenyl, C₂₋₆ alkynyl or GalNAc-containing chain, which is optionally deuterated or fully deuterated;
each R_{T2} is independently selected from H, D, halogen, CN, C₁₋₆ alkyl, C₁₋₆ haloalkyl, C₂₋₆ alkenyl or C₂₋₆ alkynyl, which is optionally deuterated or fully deuterated;
m is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, halogen, C₁₋₆ alkyl or C₁₋₆ haloalkyl, which is optionally deuterated or fully deuterated;
L is —Ar—(CH₂)₁₋₆—O—, wherein each CH₂ may optionally be substituted with R #; R # is selected from H, D, halogen, CN, C₁₋₆ alkyl, C₁₋₆ haloalkyl, C₂₋₆ alkenyl or C₂₋₆ alkynyl, which is optionally deuterated or fully deuterated;
Ar in L is linked to X; the oxygen atom in L is linked to the phosphorus atom;
Ar is selected from C₃₋₁₀ cycloalkyl, 3-10-membered heterocyclyl, C₆₋₁₀ aryl or 5-14-membered heteroaryl; C₃₋₁₀ cycloalkyl, 3-10-membered heterocyclyl, C₆₋₁₀ aryl or 5-10-membered heteroaryl may optionally be substituted with 1, 2, 3, 4 or 5 R*;
R* is selected from H, D, halogen, CN, C₁₋₆ alkyl, C₁₋₆ haloalkyl, C₂₋₆ alkenyl or C₂₋₆ alkynyl, which is optionally deuterated or fully deuterated.

7. The oligonucleotide of claim 6, wherein, [symbol]
is selected from ORₐ or NR_{b}R_{c}, or represents a bond connecting to a hydroxyl or a sulfydryl of the 5′ carbon of the ribose of an adjacent nucleotide;
X₁ is a bond connecting to a hydroxyl or a sulfydryl of the 2′ or 3′ carbon of the ribose of the other adjacent nucleotide;
Rₐ is selected from H, C₁₋₄ alkyl or C₁₋₄ haloalkyl, which is optionally deuterated or fully deuterated;
R_{b} and R_{c} is independently selected from H, C₁₋₄ alkyl or C₁₋₄ haloalkyl; and R_{b} and R_{c} may optionally be substituted with D, C₆₋₁₀ aryl, which is optionally deuterated or fully deuterated;
X₂ and X₃ is independently selected from O or S;
T is selected from 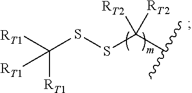
each R_{T1} is independently selected from H, D, C₁₋₄ alkyl, C₁₋₄ haloalkyl or GalNAc-containing chain, which is optionally deuterated or fully deuterated;
each R_{T2} is independently selected from H, D, C₁₋₄ alkyl or C₁₋₄ haloalkyl, which is optionally deuterated or fully deuterated;
m is 0, 1, 2 or 3;
X is selected from bond, —O—, —S—, —OC(O)NR_{X1}—, —NR_{X1}C(O)O—, —C(O)O—, —OC(O)—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is —Ar—(CH₂)—O—, wherein CH₂ may optionally be substituted with 1 or 2 R #; R # is selected from H, D, C₁₋₄ alkyl or C₁₋₄ haloalkyl, which is optionally deuterated or fully deuterated;
Ar in L is linked to X; the oxygen atom in L is linked to the phosphorus atom;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₄ alkyl or C₁₋₄ haloalkyl, which is optionally deuterated or fully deuterated;
alternatively, [symbol]
is selected from ORₐ or NR_{b}R_{c}, or represents a bond connecting to a hydroxyl or a sulfydryl of the 5′ carbon of the ribose of an adjacent nucleotide;
X₁ is a bond connecting to a hydroxyl or a sulfydryl of the 2′ or 3′ carbon of the ribose of the other adjacent nucleotide;
Rₐ is selected from H, C₁₋₄ alkyl or C₁₋₄ haloalkyl, which is optionally deuterated or fully deuterated;
R_{b} and R_{c} are independently selected from H, C₁₋₄ alkyl or C₁₋₄ haloalkyl; R_{b} and R_{c} are optionally substituted with D, phenyl, or fully deuterated;
X₃ is independently selected from O or S;
T is selected from 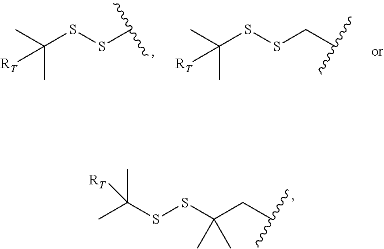
wherein R_{T} is selected from H, D, CH₃ or GalNAc-containing chain, which is optionally deuterated or fully deuterated;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is selected from 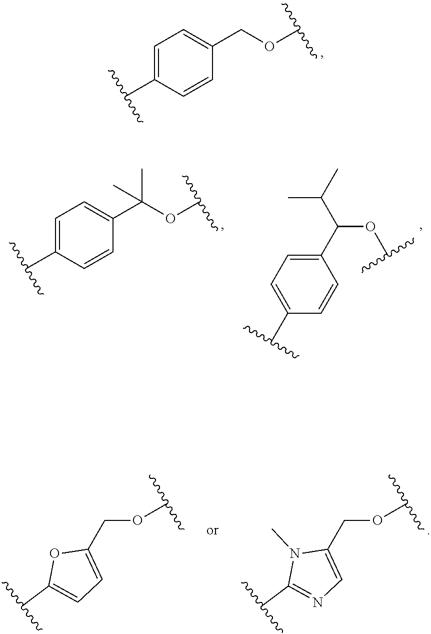
alternatively, [symbol]
represents a bond connecting to a hydroxyl or a sulfydryl of the 5′ carbon of the ribose of an adjacent nucleotide;
X₁ is a bond connecting to a hydroxyl or a sulfydryl of the 2′ or 3′ carbon of the ribose of the other adjacent nucleotide;
X₃ is selected from O or S;
T is selected from 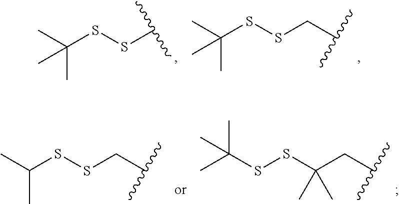
X is selected from bond, —O—, —NHC(O)O—, —OC(O)NH—, —N(CH₃)C(O)O— or —C(O)O—;
L is selected from

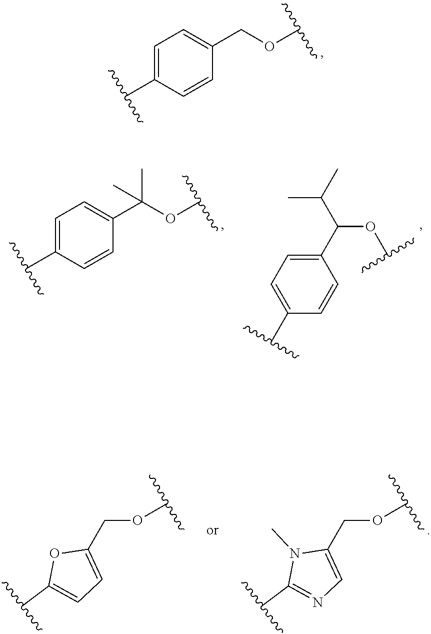

8. (canceled)

9. (canceled)

10. The oligonucleotide of claim 6, wherein the compound of formula (Ia) is selected from: 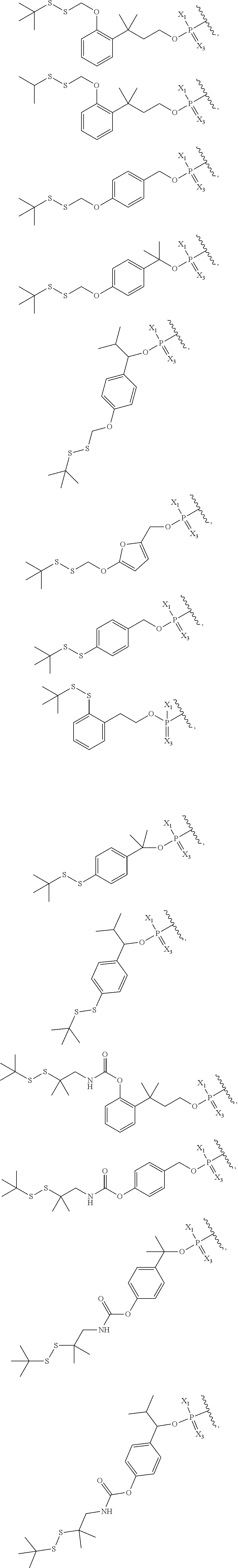 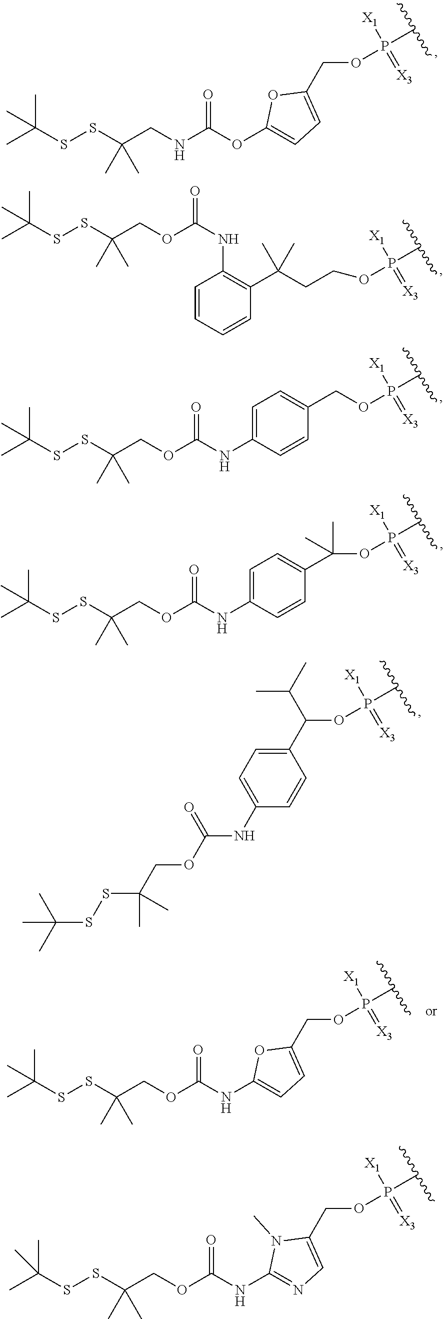
wherein, [symbol]
represents a bond connecting to a hydroxyl or a sulfydryl of the 5′ carbon of the ribose of an adjacent nucleotide;
X₁ is a bond connecting to a hydroxyl or a sulfydryl of the 2′ or 3′ carbon of the ribose of the other adjacent nucleotide;
X₃ is selected from O or S;
preferably, the compound of formula (Ia) is selected from: 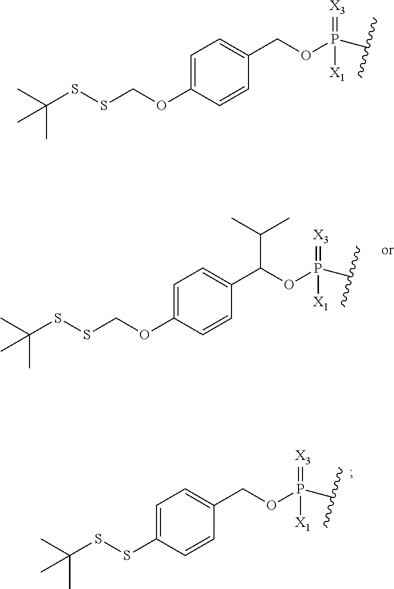 [symbol]
represents a bond connecting to a hydroxyl or a sulfydryl of the 5′ carbon of the ribose of an adjacent nucleotide;
X₁ is a bond connecting to a hydroxyl or a sulfydryl of the 2′ or 3′ carbon of the ribose of the other adjacent nucleotide;
X₃ is selected from O or S.

11. The oligonucleotide of claim 6, having 14 to 30 nucleotides.

12. The oligonucleotide of claim 6, comprising one or more compounds of formula (Ia) in the internal positions of the oligonucleotide, or a pharmaceutically acceptable salt, tautomer or stereoisomer thereof.

13. An dsRNA, comprising a sense strand an an antisense strand, wherein each strand has 14 to 30 nucleotide, wherein the antisense strand comprise a sequence sufficiently complementary to the sense strand and target mRNA, wherein the sense strand and/or the antisense strand comprise one or more compounds of formula (II) of claim 1, or a pharmaceutically acceptable salt, tautomer or stereoisomer thereof;
alternatively,
the antisense strand comprises the compound of formula (II) of claim 1 at the 5′ end, or a pharmaceutically acceptable salt, tautomer or stereoisomer thereof.

14-16. (canceled)

17. The dsRNA of claim 13, which is selected from small interfering RNA (siRNA) or small hairpin RNA (shRNA).

18. A cell comprising the dsRNA of claim 13.

19. A pharmaceutical composition comprising the dsRNA of claim 13, and optionally a pharmaceutically acceptable carrier or excipient.

20. A kit comprising the dsRNA of claim 13.

21. A compound of formula (IIb), or a pharmaceutically acceptable salt, tautomer or stereoisomer thereof: 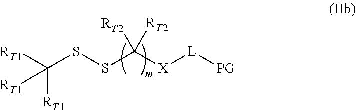
wherein,
each R_{T1} is independently selected from H, D, C₁₋₆ alkyl, C₁₋₆ haloalkyl, C₂₋₆ alkenyl, C₂₋₆ alkynyl or GalNAc-containing chain, which is optionally deuterated or fully deuterated;
each R_{T2} is independently selected from H, D, C₁₋₆ alkyl, C₁₋₆ haloalkyl, C₂₋₆ alkenyl or C₂₋₆ alkynyl, which is optionally deuterated or fully deuterated;
m is 0, 1, 2, 3, 4 or 5;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₆ alkyl or C₁₋₆ haloalkyl, which is optionally deuterated or fully deuterated;
L is —Ar—(CH₂)—O—, wherein CH₂ may optionally be substituted with 1 or 2 R #; R # is selected from H, D, C₁₋₆ alkyl or C₁₋₆ haloalkyl, which is optionally deuterated or fully deuterated;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₆ alkyl, C₁₋₆ haloalkyl, C₂₋₆ alkenyl or C₂₋₆ alkynyl, which is optionally deuterated or fully deuterated;
PG is selected from a protecting group.

22. The compound of claim 21, or a pharmaceutically acceptable salt, tautomer or stereoisomer thereof, wherein,
each R_{T1} is independently selected from H, D, C₁₋₆ alkyl, C₁₋₆ haloalkyl or GalNAc-containing chain, which is optionally deuterated or fully deuterated;
each R_{T2} is independently selected from H, D, C₁₋₆ alkyl, C₁₋₆ haloalkyl, which is optionally deuterated or fully deuterated;
m is 0, 1, 2 or 3;
X is selected from bond, —O—, —S—, —C(O)—, —C(O)O—, —OC(O)—, —OC(O)NR_{X1}—, —NR_{X1}C(O)O—, —NR_{X1}C(O)— or —C(O)NR_{X1}—;
R_{X1} is selected from H, C₁₋₄ alkyl or C₁₋₄ haloalkyl, which is optionally deuterated or fully deuterated;
L is —Ar—(CH₂)—O—, wherein CH₂ may optionally be substituted with 1 or 2 R #; R # is selected from H, D, C₁₋₄ alkyl or C₁₋₄ haloalkyl, which is optionally deuterated or fully deuterated;
Ar is selected from C₆₋₁₀ aryl or 5-10-membered heteroaryl; C₆₋₁₀ aryl or 5-10-membered heteroaryl may optionally be substituted with 1, 2 or 3 R*;
R* is selected from C₁₋₄ alkyl or C₁₋₄ haloalkyl, which is optionally deuterated or fully deuterated;
PG is selected from a protecting group;
alternatively,
PG is selected from a hydroxyl protecting group, e.g., trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), benzyloxycarbonyl (Cbz), tert-butoxycarbonyl (Boc), benzyl (Bn), p-methoxybenzyl (PMB), allyl, triphenylmethyl (Tr), di-p-methoxytrityl (DMTr), methoxymethyl (MOM), benzyloxymethyl (BOM), 2,2,2-trichloroethoxymethyl, 2-methoxyethoxymethyl (MEM), methylthiomethyl (MTM), p-methoxybenzyloxymethyl (PMBM), —C(O)CH₂CH₂C(O)OH or 4,4′-dimethoxytrityl, preferably —C(O)CH₂CH₂C(O)OH or 4,4′-dimethoxytrityl, more preferably —C(O)CH₂CH₂C(O)OH.

23. (canceled)

24. The compound of claim 21, or a pharmaceutically acceptable salt, tautomer or stereoisomer thereof, wherein, the compound of formula (IIb) is selected from: 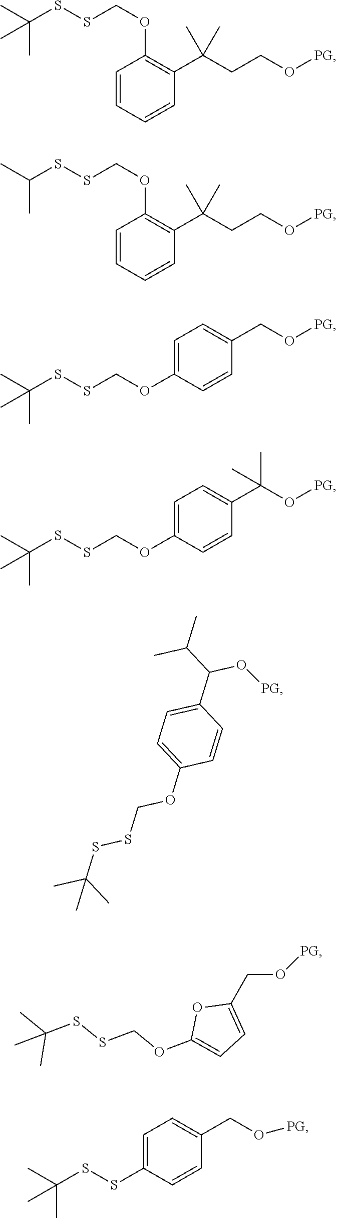 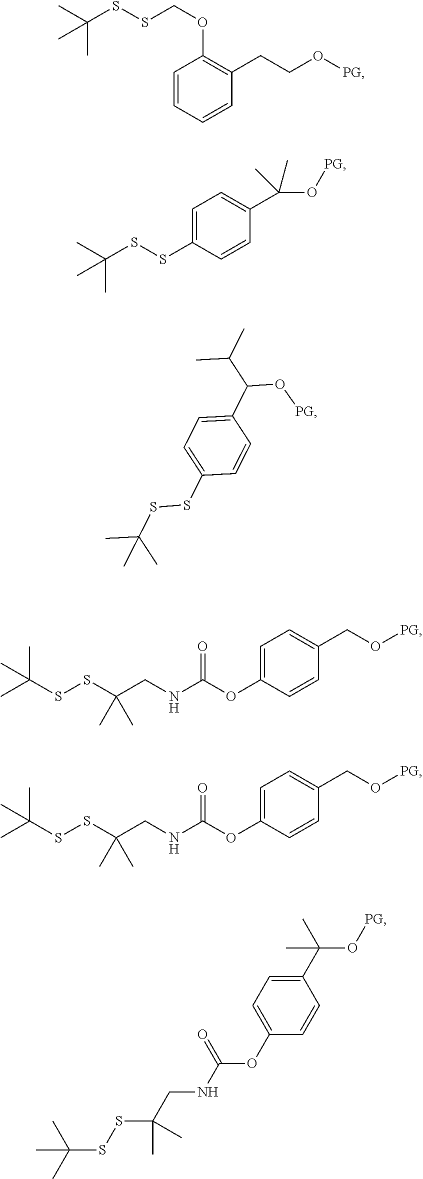 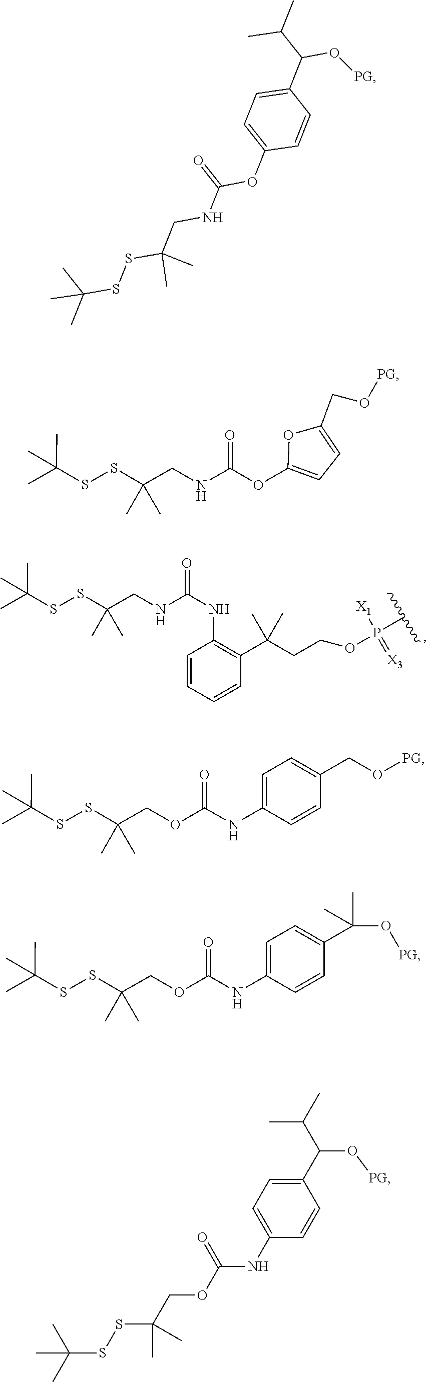 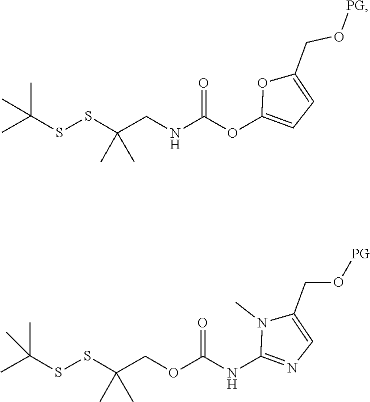
wherein,
PG is —P(OCH₂CH₂CN)(N(iPr)₂) or H.

25. An dsRNA, comprising a sense strand an antisense strand, wherein each strand has 14 to 30 nucleotide, wherein the antisense strand comprise a sequence sufficiently complementary to the sense strand and target mRNA, wherein the sense strand and/or the antisense strand comprise one or more compound of formula (Ia) of claim 6, or a pharmaceutically acceptable salt, tautomer or stereoisomer thereof;
alternatively,
the sense strand comprises one or more compounds of formula (Ia) of claim 6 in the internal positions of the oligonucleotide, or a pharmaceutically acceptable salt, tautomer or stereoisomer thereof;
alternatively,
the antisense strand comprises one or more compounds of formula (Ia) of claim 6 in the internal positions of the oligonucleotide, or a pharmaceutically acceptable salt, tautomer or stereoisomer thereof.

26. The dsRNA of claim 25, wherein the dsRNA is selected from small interfering RNA (siRNA) or small hairpin RNA (shRNA).
